SEQUENCE LISTING (1 sequence)

SEQ ID NO: 1 - protein, 333 aa, synthetic construct
MRGSHHHHHHGMASMTGGQQMGRDLYDDDDKDRWGSMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

(19) United States
(12) Patent Application Publication
(10) Pub. No.: US 2024/0239818 A1
(43) Pub. Date: Jul. 18, 2024

(54) COMPOUNDS AND COMPOSITIONS COMPRISING FLUOROPHORES FOR USE IN VISUALIZATION AND PURIFICATION OR MANIPULATION

(71) Applicant: HOWARD HUGHES MEDICAL INSTITUTE, Chevy Chase, MD (US)

(72) Inventors: Luke D. LAVIS, Ashburn, VA (US); Pratik Kumar, Ashburn, VA (US); Carla Nicolini, Ashburn, VA (US); Jonathan B. Grimm, Ashburn, VA (US)

(21) Appl. No.: 18/390,289

(22) Filed: Dec. 20, 2023

Related U.S. Application Data
(60) Provisional application No. 63/476,193, filed on Dec. 20, 2022.

(51) Int. Cl. C07F 7/08; C07D 495/04; C07K 1/22; G01N 21/64

(52) U.S. Cl. CPC C07F 7/0812; C07D 495/04; C07K 1/22; G01N 21/6486

(57) ABSTRACT

The application discloses a compound having a moiety that is either an affinity tag-containing moiety or a protein-manipulation moiety, a self-labeling protein (SLP) ligand, and a rhodamine dye linking the affinity tag-containing moiety or a protein-manipulation moiety to the SLP ligand. Also disclosed is a complex, which includes the compound and a SLP. Also disclosed is a method that involves contacting the compound and a SLP with a cell, and visualizing fluorescence in the cell or purifying the SLP and associated biological components from the cell.

### RELATED APPLICATIONS

This application claims priority from U.S. Provisional Application Ser. No. 63/476, 193 filed Dec. 20, 2022, the entire disclosure of which is incorporated herein by this reference.

### REFERENCE TO AN ELECTRONIC SEQUENCE LISTING

The contents of the electronic sequence listing (Lavis 23001US Sequence Listing.xml; Size: 2,363 bytes; and Date of Creation: Dec. 15, 2023) is herein incorporated by reference in its entirety.

### TECHNICAL FIELD

The presently-disclosed subject matter generally relates to fluorescent compounds. In particular, certain embodiments of the presently disclosed subject matter related to multifunctional fluorophores that can be used for visualization in live cells and purification or manipulation.

### INTRODUCTION

Advances in the understanding of biological processes rely on the ability to visualize, purify, and manipulate cellular components. Research at the interface of synthetic organic chemistry and protein biochemistry has generated powerful tools to probe and perturb cells with molecular specificity.¹⁻³ In particular, the engineering of enzyme-substrate interactions produced self-labeling tags such as the HaloTag®⁴ and SNAP-tag®.⁵ These protein tags can react specifically and irreversibly with compounds bearing the self-labeling tag ligand.⁶⁻¹⁰ For example, appending polyethylene glycol (PEG) containing 1-chloroalkane moiety to 6-carboxytetramethylrhodamine (TMR) affords TMR-HaloTag® ligand (1, FIG. 1A). This compound (1) is cell-permeable, exhibits rapid labeling kinetics with the HaloTag®, and upon labeling positions the TMR moiety close the surface of HaloTag® protein (FIG. 1B; PDB:6U32)¹¹. This intimate association of rhodamine and HaloTag® can elicit further improvements in fluorophore photophysics.¹² The generality of this tagging with different rhodamines has rekindled the development of rhodamines with improved photophysical properties and cellular permeabilities to meet the demand of modern fluorescence microscopy.

A key property of rhodamines influencing their performance in the biological systems is their equilibrium between the non-fluorescent and lipophilic lactone (L) and fluorescent zwitterion (Z) (FIG. 1C). It was previously shown that the lactone-zwitterion equilibrium constant (K_{L-Z}) can be a reliable proxy of rhodamines performance.¹³ Dyes with high K_{L-Z} such as Janelia Fluor 549 (JF₅₄₉, 2) predominantly exist in the zwitterionic form irrespective of the environment. These dyes have high absorptivity that is insensitive to HaloTag® labeling, making them always bright. Dyes with moderate K_{L, Z} such as the carborhodamine JF₆₀₈ (3) prefer the zwitterionic form in aqueous media but shift to the lactone form in hydrophobic environment. These dyes easily permeate through the cellular membranes. Dyes with slightly lower K_{L-Z} such as silicon-rhodamines JF₆₄₆ (4) and JF₆₃₅ (5) predominantly exist in the lactone form in aqueous media but shift to more zwitterionic form upon binding to the HaloTag® protein. These dyes make good environmentally sensitive labels, making them highly cell-permeable and fluorogenic. Lastly, dyes with very low K_{L-Z} preferentially adopt the lactone form, making them colorless and unsuitable for biological imaging. Combining this equilibrium of rhodamines with self-labeling tags have yielded hybrid small-molecule-protein indicators of cellular activity.^{11, 14-16}

The simplicity of self-labeling tags has also sparked the development of “rhodamine-less” reagents for purifying or manipulating the biological systems. For such reagents, however, the intimate association of HaloTag® protein and ligand can be detrimental, requiring longer linkers that can affect cell-permeability, labeling kinetics, and the properties of the resulting protein conjugate.^{7-9, 17} For example, the commercial HaloTag® ligands containing biotin are unsuitable for streptavidin mediated affinity purification of HaloTag® fused intracellular proteins (discussed below). Given the common tug-of-war between functionality, kinetics, and permeability in small-molecule ligand development, the K_{L-Z} equilibria of rhodamines was sought to be exploited to use them as linkers for generating cell-permeable “multifunctional” reagents that enable protein purification or manipulation in addition to fluorescence visualization of HaloTag® fused proteins (FIG. 1D).

Accordingly, there remains a need in the art for multifunctional fluorophores that can be used for visualization in live cells, as well as modification and/or subsequent affinity capture.

### SUMMARY

The presently-disclosed subject matter meets some or all of the above-identified needs, as will become evident to those of ordinary skill in the art after a study of information provided in this document.

A summary of the presently-disclosed subject matter will first be described in connection with exemplary embodiments of the compounds and methods, and further reference is made to the Examples provided herein.

The present-disclosed subject matter includes a compound generally having three components, (1) a moiety that is either an affinity tag-containing moiety or a protein-manipulation moiety; (2) a self-labeling protein (SLP) ligand; and (3) a rhodamine dye linking the affinity tag-containing moiety or a protein-manipulation moiety to the SLP ligand.

In some embodiments, the compound has the following formula:

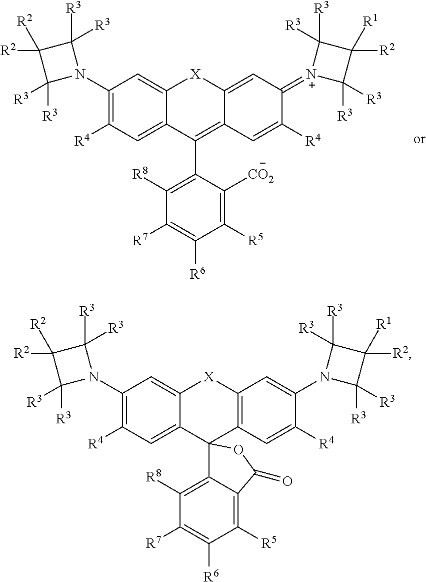

or a salt thereof, in which X is O, C(CH₃)₂, or Si(CH₃)₂; R¹ is an affinity tag-containing moiety or a protein-manipulation moiety; each R² is independently selected from the group consisting of H, D, halogen, OH, O(alkyl), N(alkyl)₂, CF₃, CN, COOH, COO(alkyl), C(O)NH(alkyl), C(O)N(alkyl)₂, and SO₂CH₃; each R³ is independently selected from the group consisting of H and D; each R⁴ is independently selected from the group consisting of H, halogen, CF₃, and CN; and R⁵, R⁶, R⁷, and R⁸ are each independently selected from the group consisting of H, F, CO₂H, and a self-labeling protein (SLP) ligand, so long as one of R⁵, R⁶, R⁷, and R⁸ is the SLP ligand.

The presently-disclosed subject matter further includes a complex, which comprises a compound as disclosed herein, and further comprising a self-labeling protein (SLP).

The presently-disclosed subject matter further includes a method, which comprises contacting a compound as disclosed herein and a self-labeling protein (SLP) with a cell; and visualizing fluorescence in the cell. The presently-disclosed subject matter further includes a method, which comprises contacting a compound as disclosed herein and a self-labeling protein (SLP) with a cell; and purifying the SLP and associated biological components from the cell.

Inserting rhodamines as linkers was predicated on several factors. First, the directed evolution of the SLP, HaloTag® protein, utilized rhodamine compound 1⁴ and rhodamine-based HaloTag® ligands show rapid labeling kinetics even when additional functionality is added to the dye moiety.¹⁷ Second, improvements in dye chemistry enable the construction of “multifunctional fluorophores” where different moieties are attached to the dye for labeling,¹⁸ or sensing^{14, 16, 19-22} applications. Third, the ability to fine-tune the chemical and spectral properties of rhodamine dyes^{13, 23-27} could allow optimization of the cell-permeability or other properties of the entire ligand. Fourth, addition of HaloTag® ligand on dyes 2-5 to obtain 2_{HTL}-5_{HTL} (FIG. 2) has minimal change in K_{L-Z}, and their distribution coefficients at pH 7.4 (logD_{7.4}) is inversely correlated with K_{L-Z} (FIG. 1E and discussed below); suggesting that dyes with low K_{L-Z} could drive the permeability of bulkier multifunctional reagents. Finally, the incorporation of a fluorescent dye enables visualization of the ligand inside cells, allowing live cell verification of the ligand's performance using fluorescence imaging.

As disclosed herein, the K_{L-Z} equilibria of rhodamines was exploited to use them as linkers for the polar biotin (clogP=−1.28) and the non-polar pharmacophore JQ1 (clogP=1.79) for live cell labeling of HaloTag® fusion proteins. The biotin ligand allows affinity pulldown and JQ1 ligand allows protein manipulation apart from live cell visualization of the ligand. For biotin ligands, live cell permeable and cell impermeable rhodamine-containing biotin ligands with distinct spectral properties are described herein. Their ability to label HaloTag® fusion proteins was tested at four different cellular locations (cell surface, mitochondria, endoplasmic reticulum, and nucleus) and in three different mammalian cell lines (HEK293T, U2OS), and verified their ability to affinity purify intracellular proteins with the two fluorogenic versions. For JQ1 ligands, two fluorogenic JQ1 ligands are reported for three color “no-wash” imaging of three sub-nuclear HaloTag® fusion proteins (coilin, heterochromatin protein 1, and centromere protein A), and subsequent BRD4 recruitment to them in N2a cells. Given the broad utility of biotin conjugates in biochemistry and cell biology, and JQ1 analogs in disease biology, these ligands will be broadly useful in live-cell context. The interplay between the K_{L-Z} and logD_{7.4} to use “rhodamine-as-linker” is applicable to other small ligands, providing for biologically useful cell-permeable tools that go beyond fluorescence visualization. Such reagents enable live cell visualization of the ligand, which is not possible with reagents with low cell permeability or lacking fluorophores, to enable new questions in biology.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A-1E. Rhodamines linkers for self-labeling tags. FIG. 1A—Chemical structure of 1. FIG. 1B—Crystal structure of 1 covalently bound to the HaloTag® protein (PDB:6U32) with zoom-in of the dye-protein interface. FIG. 1C—General rhodamine structure showing the dynamic equilibrium between the nonfluorescent lactone (L) and fluorescent zwitterion (Z) along with the lactone-zwitterion equilibrium constant (K_{L-Z}) for Janelia Fluor (JF) dyes 2-5. K_{L-Z} values are in log scale. FIG. 1D—Schematic illustrating the use of a rhodamine linker to create multifunctional fluorophores that enable live-cell fluorescence microscopy and affinity capture or manipulation. FIG. 1E—Plot of logD_{7.4} and K_{L-Z} for dyes 2-5 and their HaloTag® ligands (2_{HTL}-5_{HTL}). Shading reflects expected cellular permeability based on logD_{7.4}.

FIG. 2. Chemical structures of HaloTag® ligands of JF dyes 2-5.

FIG. 3A-3D. Performance of biotin-HaloTag® ligand (2). FIG. 3A—Schematic of the assay to evaluate biotin-HaloTag® conjugates starting with incubation with live HEK293T cells followed by purification using streptavidin affinity reagents. FIG. 3B—Chemical structure of commercially available biotin-HaloTag® ligand (6). FIGS. 3C & 3D—SDS-PAGE/in-gel fluorescence (FIG. 3C) and quantification (FIG. 3D) showing the amount of msGFP-HaloTag® fusion protein bound to streptavidin using ligand 6; error bars represent ±SEM. (n=3); “total/20” indicates the lane was loaded with a 1:20 (v/v) dilution of the crude sample containing total protein for quantification purposes.

FIG. 4A-4D. Evaluation of cellular permeability of biotin-HaloTag® ligand (6). FIG. 4A—Schematic of the experiment to determine the permeability of biotin-HaloTag® ligand (6) measuring either streptavidin capture efficiency or a pulse-chase experiment with fluorescent JF₅₄₉-HaloTag® ligand (2_{HTL}). FIG. 4B—Schematics and corresponding chemical structures of 6 and 2_{HTL}. FIG. 4C—Ratio of JF₅₄₉/msGFP fluorescence in HEK293T cells expressing msGFP-HaloTag® fusion localized to the mitochondrial outer membrane after incubation with 2_{HTL} only (100 nM; gray circles) or after a pulse-change protocol where cells were first incubated with biotin-HaloTag® ligand (6; 100 nM or 10 μM) followed by incubation with 2_{HTL} (magenta circles). Incubation with 10 μM of 6 substantially labeled the HaloTag® protein fusion, leading to a low signal from the JF₅₄₉ ligand 2_{HTL} chase (n>30). FIG. 4D—Q-Q plot of the data from c showing a normal distribution.

FIG. 5A-5C. Design and properties of rhodamine containing biotin ligands. FIG. 5A—Synthesis of biotin-JF-HaloTag® ligands 12-15 from 3″-carboxyazetidine dyes 7-10 and biotin amine 11. FIG. 5B—Spectral properties of 2-5, 12-15 and their HaloTag® conjugates. All measurements taken in 10 mM HEPES, PH 7.3. ^{a}Data for 2-5 taken from refs.²³ and³². λ_{abs}/λₑₘ and ε are in nm and M⁻¹cm⁻¹ respectively. FIG. 5C—Chemical properties of 2-5, 2_{HTL}-5_{HTL}, and 12-15. ^{b}K_{L-Z} measurements were performed in 1:1 (v/v) dioxane-water. ^{c}K_{L-Z} data for 2-5 taken from ref.²³

FIG. 6. Plot of logD_{7.4} and K_{L-Z} for JF dyes (2-5), their HaloTag® ligands (2_{HTL}-5_{HTL}), and their biotin analogs (12-15). Shading reflects expected cellular permeability based on logD_{7.4}.

FIG. 7. Evaluation of biotin-rhodamine-HaloTag® ligands for live-cell labeling of HaloTag® fusions at different cellular locations. Airyscan fluorescence microscopy images of U2OS cells expressing HaloTag® fusion proteins and incubated with ligands 12-15; cells were fixed before imaging. (Panels A-D) Cell surfaced-localized HaloTag-PDGFR fusion. (Panels E-H) Outer mitochondrial membrane-localized HaloTag-TOMM20 fusion. (I-L) Endoplasmic reticulum membrane-localized HaloTag-Sec61β fusion. (M-P) Nucleus-localized HaloTag-histone H2B fusion. Cells were imaged after incubation with 100 nM for 1 h with biotin-JF₅₄₉-HaloTag® ligand (12; Panels A, E, I, M), biotin-JF₆₀₈-HaloTag® ligand (13; Panels B, F, J, N), biotin-JF₆₄₆-HaloTag® ligand (14; Panels C, G, K, O), or biotin-JF₆₃₅-HaloTag® ligand (15; Panels D, H, L, P) followed by fixation and counterstaining with Hoechst 33342. Image sets in Panels A/E/I/M, Panels B/F/J/N, Panels C/G/K/O, and Panels D/H/L/P each used the same microscope settings. Scale bars for all images: 10 μm

FIG. 8A-8E. Fluorescence imaging of HaloTag® fusion with biotin-JF₅₄₉-HaloTag® ligand (12). (A) Chemical structures of the JF₅₄₉-HaloTag® ligand (2_{HTL}) and biotin-JF₅₄₉-HaloTag® ligand (12). (B-E) Fluorescence microscopy images of live HEK293T cells expressing msGFP-HaloTag® fusion proteins localized to the mitochondrial outer membrane after incubation with either JF₅₄₉-HaloTag® ligand (2_{HTL}; B,C) or biotin-JF₅₄₉-HaloTag® ligand (12; D,E) and counterstained with Hoechst 33342. Images in B and D were taken with the same microscope settings. Scale bars: 10 μm.

FIG. 9. Evaluation of cellular permeability of biotin-JF₅₄₉-HaloTag® ligand (12). Ratio of JF₅₄₉/msGFP fluorescence in HEK293T cells expressing msGFP-HaloTag® fusion localized to the mitochondrial outer membrane after incubation with JF₅₄₉-HaloTag® ligand (2_{HTL}; 100 nM) or biotin-JF₅₄₉-HaloTag® ligand (12; 100 nM).

FIG. 10. Loading curves for biotin-JF-HaloTag® ligands (12-15) in live U2OS cells.

FIG. 11A-11N. Evaluation of biotin-JF₆₄₆-HaloTag® ligand (14) and biotin-JF₆₃₅-HaloTag® ligand (15) for affinity purification. FIG. 11A—Chemical structures of biotin-JF₆₄₆-HaloTag® ligand (14) and parent ligand JF₆₄₆-HaloTag® ligand (4_{HTL}). FIG. 11B-11E—Airyscan fluorescence microscopy images of HEK293T cells expressing msGFP-HaloTag® fusion localized to the mitochondrial outer membrane after incubation with either JF₆₄₆-HaloTag® ligand (4_{HTL}; FIG. 11B, 11C) or biotin-JF₆₄₆-HaloTag® ligand (14; FIG. 11D, 11E) and counterstained with Hoechst 33342; cells were fixed before imaging. (f) Chemical structures of biotin-JF₆₃₅-HaloTag® ligand (15) and parent ligand JF₆₃₅-HaloTag® ligand (5_{HTL}). FIG. 11G-11J—Airyscan fluorescence microscopy images of HEK293T cells expressing msGFP-HaloTag® fusion localized to the mitochondrial outer membrane after incubation with either JF₆₃₅-HaloTag® ligand (5_{HTL}; FIG. 11G, 11H) or biotin-JF₆₃₅-HaloTag® ligand (15; FIG. 11I, 11J) and counterstained with Hoechst 33342; cells were fixed before imaging. Image sets FIG. 11B/D/G/I and FIG. 11C/E/H/J each used the same microscope settings; scale bars: 10 μm. FIG. 11K—Plot of the ratio of JF₆₄₆ or JF₆₃₅ cellular fluorescence intensity and msGFP cellular fluorescence intensity; n=36 (4_{HTL}), n=31 (14), n=30 (5_{HTL}), and n=36 (15), taken from three independent experiments per compound with each n representing a delineated cell. FIG. 11L—Absorbance of 14 or 15 in the absence or presence (+HT) of excess HaloTag® protein. FIG. 11M, 11N—SDS-PAGE/in-gel fluorescence (FIG. 11M) and quantification (FIG. 11N) measuring the amount of msGFP fusion protein bound to streptavidin after labeling with ligands 5_{HTL}, 14, or 15; “total/20” indicates the lane was loaded with a 1:20 dilution (v/v) of the crude sample containing total protein; error bars represent ±SEM (n=3). Significance testing was performed using two-way ANOVA and Tukey's multiple comparisons test with a single pooled variance; ns indicates not significant; **** indicates P<0.0001

FIG. 12A-12B. Design and properties of rhodamine containing biotin ligands. FIG. 12A—Synthesis of (+)-JQ1-JF-HaloTag® ligands 17-18 from 3″-carboxyazetidine dyes 9-10 and (+)-JQ1 amine 16. FIG. 12B—Spectral and chemical properties of 17-18 and their HaloTag® conjugates. All measurements taken in 10 mM HEPES, PH 7.3. λ_{abs}/λₑₘ and ε are in nm and M⁻¹cm⁻¹ respectively. K_{L-Z} measurements were performed in 1:1 (v/v) dioxane-water.

FIG. 13A-13J. Evaluation of JQ1-rhodamine-HaloTag® ligands (18, 19) for live-cell protein manipulation. (A) Schematic illustrating the use of a rhodamine linker to create JQ1 ligands that enable manipulation of BRD4. (B-G) Maximum intensity projections from lattice lightsheet fluorescence microscopy of live N2A cells expressing sfGFP-BRD4 and HaloTag® fusion proteins at mentioned times after incubation with 100 nM 18. (B-D) sfGFP and JF₆₃₅ fluorescence signals for cells expressing HP1-HaloTag®. (E-G) sfGFP and JF₆₃₅ fluorescence signals for cells expressing coilin-HaloTag® fusion. (H) Time-resolved fluorescent intensity quantification of sfGFP-BRD4 and (+)-JQ1-JF₆₃₅-HaloTag® ligand (18) at coilin-HaloTag® labeled with 18. (I) Side-by-side comparison of (+)-JQ1-JF₆₄₆-HaloTag® ligand (17) and 18 for recruiting BRD4 to coilin-HaloTag fusion. (J) Side-by-side comparison of H3.3 localization at coilin-HaloTag labeled with JF₆₃₅-HaloTag ligand (5_{HTL}) or 18. The dashed line represents the nuclear boundary determined through a histone probe. Scale bars for all images: 10 μm

FIG. 14A-14F. Evaluation of (+)-JQ1-JF₆₃₅-HaloTag® ligand (18) labeled HP1 for live-cell manipulation of BRD4. Maximum intensity projections from lattice lightsheet fluorescence microscopy of live N2A cells expressing sfGFP-BRD4 and HP1-HaloTag fusions at time 0 min, 52 min, and 90 min after incubation with 100 nM 18. FIG. 14A-14C—Extracted fluorescence signal from sfGFP alone. FIG. 14D-14F—Fluorescence signal from sfGFP and JF₆₃₅. The dashed line represents the nuclear boundary determined through a histone probe.

FIG. 15A-15F. Evaluation of (+)-JQ1-JF₆₃₅-HaloTag® ligand (18) labeled Coilin for live-cell manipulation of BRD4. Maximum intensity projections from lattice lightsheet fluorescence microscopy of live N2A cells expressing sfGFP-BRD4 and Coilin-HaloTag® fusions at time 0 min, 52 min, and 90 min after incubation with 100 nM 18. FIG. 15A-15C—Extracted fluorescence signal from sfGFP alone. FIG. 15D-15F—Fluorescence signal from sfGFP and JF₆₃₅. The dashed line represents the nuclear boundary determined through a histone probe.

FIG. 16A-16F. Evaluation of (−)-JQ1-JF₆₃₅-HaloTag® ligand (19) for live-cell manipulation of BRD4. Maximum intensity projections from lattice lightsheet fluorescence microscopy of live N2A cells expressing sfGFP-BRD4 and HP1-HaloTag® fusions at time 0 min, 52 min, and 90 min after incubation with 100 nM 19. FIG. 16A-16C—Extracted fluorescence signal from sfGFP alone. FIG. 16D-16F—Fluorescence signal from sfGFP and JF₆₃₅. The dashed line represents the nuclear boundary determined through a histone probe.

FIG. 17A-17F. Evaluation of (−)-JQ1-JF₆₃₅-HaloTag® ligand (19) for live-cell manipulation of BRD4. Maximum intensity projections from lattice lightsheet fluorescence microscopy of live N2A cells expressing sfGFP-BRD4 and coilin-HaloTag® fusions at time 0 min, 52 min, and 90 min after incubation with 100 nM 19. (A-C) Extracted fluorescence signal from sfGFP alone. (D-F) Fluorescence signal from sfGFP and JF₆₃₅. The dashed line represents the nuclear boundary determined through a histone probe.

FIG. 18. Chemical structure of blebbistatin-JF₆₄₆-HaloTag® ligand (24).

### BRIEF DESCRIPTION OF THE SEQUENCE LISTING

SEQ ID NO: 1 is the amino acid sequence of HaloTag® protein (HT7).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The present-disclosed subject matter includes a compound comprising a moiety that is either an affinity tag-containing moiety or a protein-manipulation moiety, a self-labeling protein (SLP) ligand, and a rhodamine dye linking the affinity tag-containing moiety or a protein-manipulation moiety to the SLP ligand.

In some embodiments, the compound has the following formula:

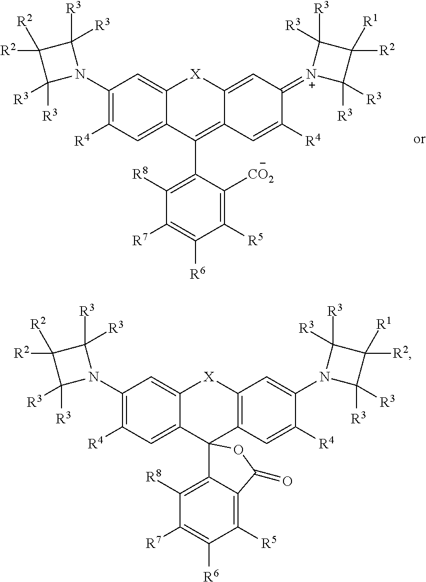

or a salt thereof, in which X is O, C(CH₃)₂, or Si(CH₃)₂; R¹ is an affinity tag-containing moiety or a protein-manipulation moiety; each R² is independently selected from the group consisting of H, D, halogen, OH, O(alkyl), N(alkyl)₂, CF₃, CN, COOH, COO(alkyl), C(O)NH(alkyl), C(O)N(alkyl)₂, and SO₂CH₃; each R³ is independently selected from the group consisting of H and D; each R⁴ is independently selected from the group consisting of H, halogen, CF₃, and CN; and R⁵, R⁶, R⁷, and R⁸ are each independently selected from the group consisting of H, F, CO₂H, and a self-labeling protein (SLP) ligand, so long as one of R⁵, R⁶, R⁷, and R⁸ is the SLP ligand.

R¹ can be an affinity tag-containing moiety or a protein-manipulation moiety. As used here, the term “affinity tag-containing moiety” refers to a moiety including an affinity tag and a linker joining the affinity tag to the rhodamine dye. The affinity tag can be any affinity tag known to those of ordinary skill in the art used for purification of proteins or other cellular components, including, but not limited to, the following examples: biotin and desthiobiotin for avidin-mediated capture, trimethoprim/folate/methotrexate for dihydrofolate reductase (DHFR)-mediated capture, a peptide epitope such as FLAG for antibody-mediated capture, or a click chemistry reagent such as azide, alkyne, tetrazine or dibenzocyclooctyne (DBCO) for biorthogonal capture. As will be appreciated by those of ordinary skill in the art, the linker joining the affinity tag to the rhodamine dye can be selected in view of the selection of the affinity tag, to provide for effective linking to the rhodamine dye. In this regard, in one example, the linker could be a polyethylene glycol (PEG) bearing a terminal amino group, an alkane bearing a terminal amino group, or a polypeptide such as polyglycine or polyproline. In other examples, the linker may contain the result of various conjugation chemistries used to synthesize the molecule, such as maleimide-thiol chemistry or azide-alkyne click chemistry.

As used here, the term “protein-manipulation moiety” refers to a moiety including a protein-manipulation ligand and a linker joining the protein-manipulation ligand to the rhodamine dye. The protein-manipulation ligand can be any ligand known to those of ordinary skill in the art to interact with a protein of interest, such as an inhibitor, allosteric binder, or activator. By way of provided some specific, non-limiting examples: (+)-JQ1 could be used to inhibit the bromodomain and extra-terminal motif (BET) family of proteins; blebbistatin could be used to inhibit myosin II; Trichostatin A (TSA) or suberoylanilide hydroxamic acid (SAHA) could be used to inhibit histone deacetylases (HDACs); MAK683 could be used to inhibit the EED regulatory subunit of the PRC2 complex. As will be appreciated by those of ordinary skill in the art, the linker joining the protein-manipulation ligand to the rhodamine dye can be selected in view of the selection of the protein-manipulation ligand, to provide for effective linking to the rhodamine dye. In this regard, in one example, the linker could be a polyethylene glycol (PEG) bearing a terminal amino group, an alkane bearing a terminal amino group, or a polypeptide such as polyglycine or polyproline. In other examples, the linker may contain the result of various conjugation chemistries used to synthesize the molecule, such as maleimide-thiol chemistry or azide-alkyne click chemistry.

In some embodiments of the compound, R¹ is

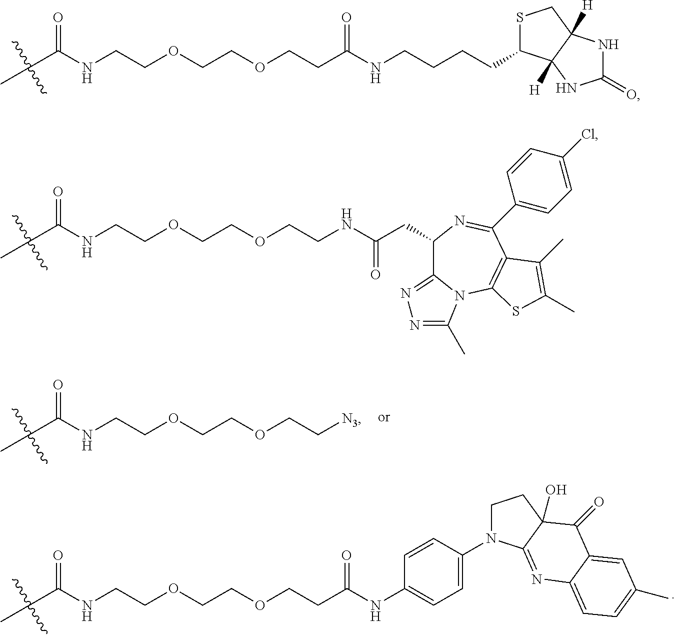 [SMILES: *C(=O)NCCOCCOCCC(=O)NCCCC[C@@H]1SC[C@@H]2NC(=O)N[C@@H]21 ; *C(=O)NCCOCCOCCNC(=O)C[C@@H]1N=C(c2ccc(Cl)cc2)c2c(sc(C)c2C)-n2c(C)nnc21 ; *C(=O)NCCOCCOCCN=[N+]=[N-] ; "or" ; *C(=O)NCCOCCOCCC(=O)Nc1ccc(N2CCC3(O)C(=O)c4cc(C)ccc4N=C23)cc1]

As will be appreciated by those of ordinary skill in the art, the terms “self-labeling tag” and “self-labeling protein tag” are interchangeably used to refer to a fusion protein system for facilitating the specific attachment of a compound to a protein tag within a living cell or in vitro. The term “self-labeling” indicates that the protein tag is capable of catalyzing the attachment to the compound without the need for additional enzymes or co-factors. A self-labeling tag system includes a “protein tag” or “self-labeling protein (SLP)” and a “protein tag ligand” or “SLP ligand.” The SLP and the SLP ligand form a specific bond. In this regard, when the SLP ligand is attached to a compound, the SLP forms a bond with the compound via the SLP ligand. This bond formation ensures a stable and irreversible attachment of the compound to the protein, allowing for various applications such as visualization, purification, and interaction studies.

Examples of self-labeling protein tags will be known to those of ordinary skill in the art and include, for example, HaloTag®. HaloTag® protein is known for its ability to form a bond with a chloroalkane ligand. Additional examples of self-labeling protein tags include SNAP-tag®, TMP-tag®, βLac-tag, CLIP-tag®, and biotin-avidin.

In some embodiments of the compound, the SLP ligand is

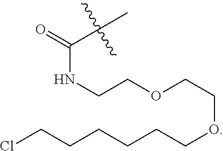 [SMILES: *C(=O)NCCOCCOCCCCCCCl]

In some embodiments of the compound, R² is H or F; R³ and R⁴ are H; R⁵, R⁶, and R⁸ are H; and R⁷ is the SLP ligand. In some embodiments of the compound, X is O, Si(CH₃)₂, or Si(CH₃)₂.

In some embodiments, the compound is of the following formula

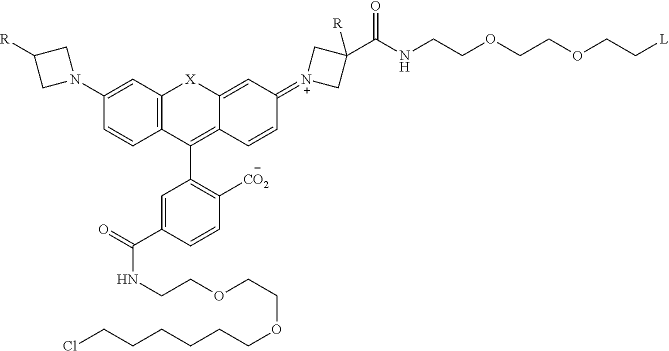

wherein R is H or F; X is O, C(CH₃)₂, or Si(CH₃)₂; and L is an affinity tag-containing moiety or a protein-manipulation moiety.

In some embodiments, the compound is of the following formula:

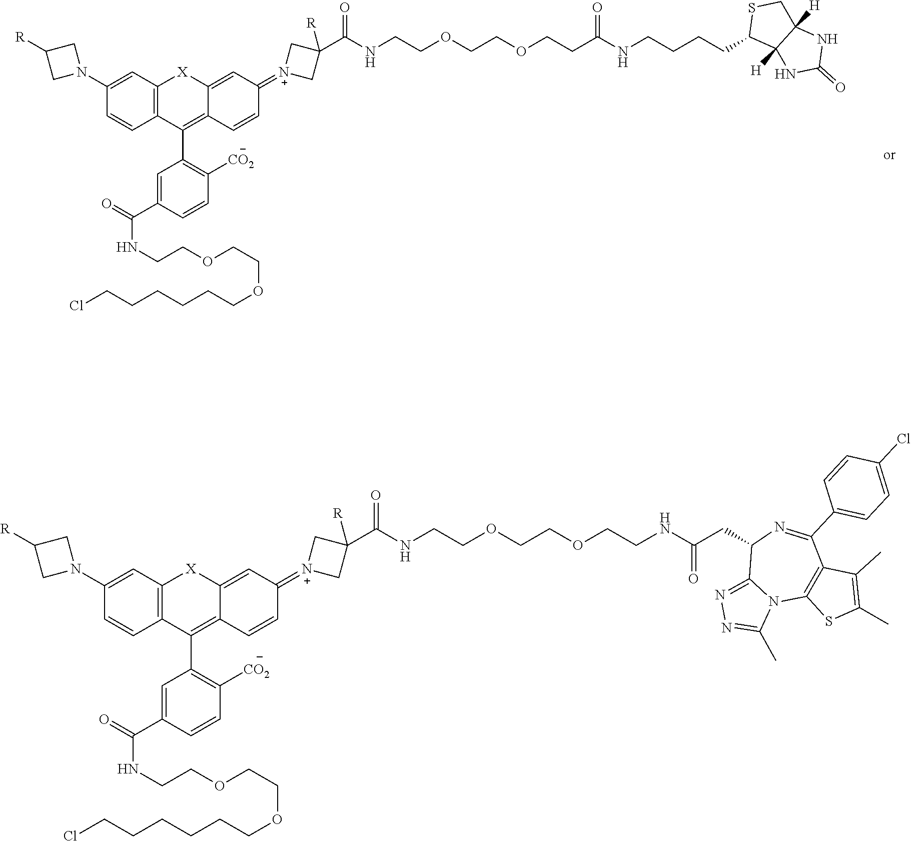

wherein R is H or F, and X is O, C(CH₃)₂, or Si(CH₃)₂.

In some embodiments, the compound is of the following formula:

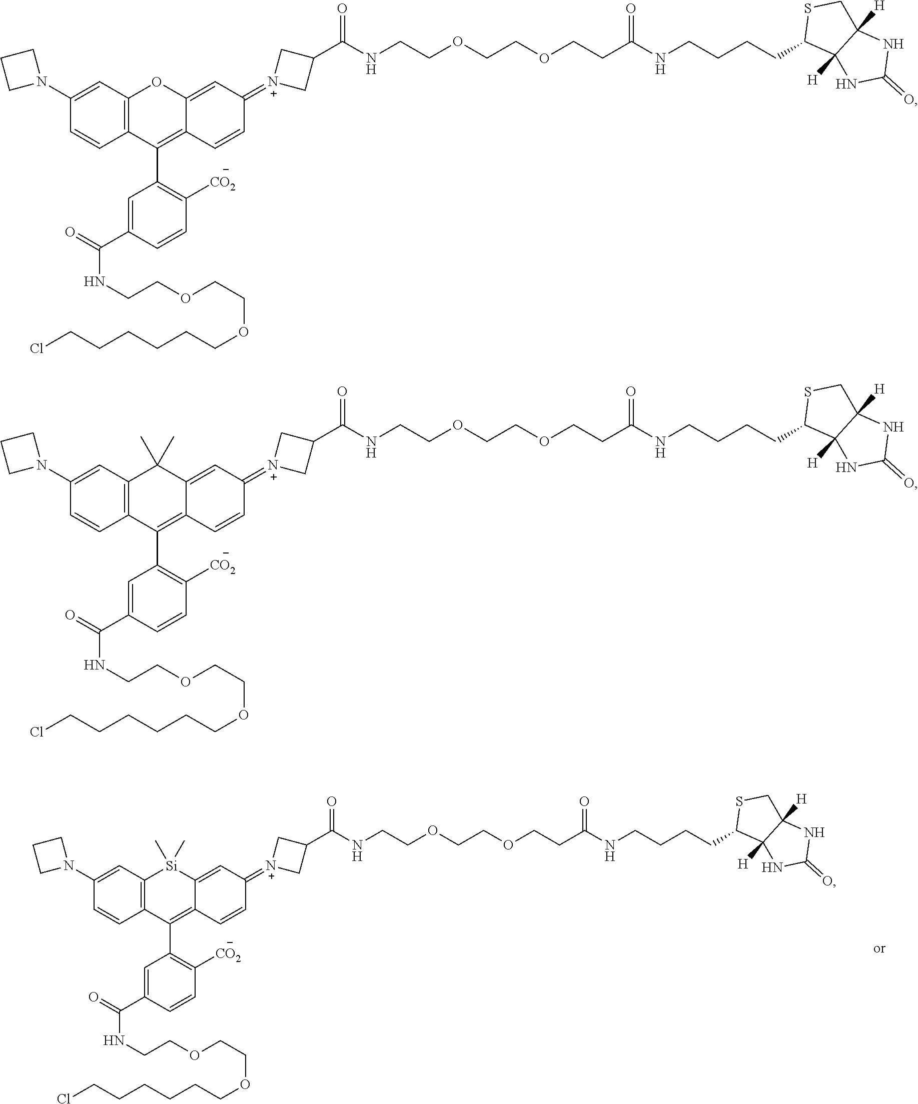

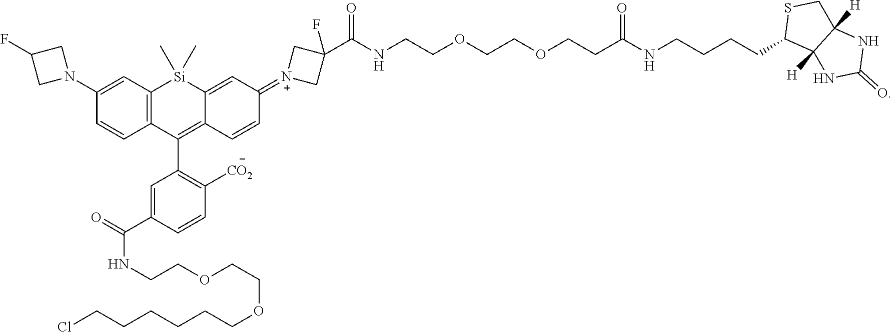

The presently-disclosed subject matter further includes a complex, which comprises a compound as disclosed herein, and further comprising a self-labeling protein (SLP).

The presently-disclosed subject matter further includes a method, which comprises contacting a compound as disclosed herein and a self-labeling protein (SLP) with a cell; and visualizing fluorescence in the cell. The presently-disclosed subject matter further includes a method, which comprises contacting a compound as disclosed herein and a self-labeling protein (SLP) with a cell; and purifying the SLP and associated biological components from the cell.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients,

properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, methylpropynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

“D” refers to deuterium.

The terms “halo,” “halide,” or “halogen” as used herein refer to fluoro, chloro, bromo, and iodo groups.

When the term “independently selected” is used, the substituents being referred to (e.g., R groups, such as groups R⁶ and R⁷), can be identical or different. For example, both R⁶ and R⁷ can be hydrogen, or R⁶ can be hydrogen and R⁷ can be a SLP ligand, and the like.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

HaloTag® protein purification. The bacterial expression vector pRSET-A (Invitrogen) was used to recombinantly express HaloTag® protein (HT7; Promega).⁴⁵ The soluble 6×His-Tagged HaloTag® protein was affinity purified by immobilized metal affinity chromatography (IMAC) on a 5-mL Fast Flow HiTrap Sepharose 6 column (Cytiva) with a 0-200 mM imidazole elution gradient using an Avant Protein Purification System (ÄKTA). A₂₈₀ peak fractions were pooled, concentrated by a spin concentrator, and dialyzed 3× into tris-buffered saline (TBS). The amino acid sequence of HaloTag® protein (HT7) expressed from pRSET-A is SEQ ID NO: 1.

SEQ ID NO: 1:
MRGSHHHHHHGMASMTGGQQMGRDLYDDDDKDRWGSMAEIGTGFP
FDPHYVEVLGERMHYVDVGPRDGTPVLFLHGNPTSSYVWRNIIPH
VAPTHRCIAPDLIGMGKSDKPDLGYFFDDHVRFMDAFIEALGLEE
VVLVIHDWGSALGFHWAKRNPERVKGIAFMEFIRPIPTWDEWPEF
ARETFQAFRTTDVGRKLIIDONVFIEGTLPMGVVRPLTEVEMDHY
REPFLNPVDREPLWRFPNELPIAGEPANIVALVEEYMDWLHQSPV
PKLLFWGTPGVLIPPAEAARLAKSLPNCKAVDIGPGLNLLQEDNP
DLIGSEIARWLSTLEISG
Underlined features are (1) 6xHis;
(2) T7tag (gene 10 leader); (3) Xpress™tag;
and (4) HaloTag®.

1-Photon spectroscopy of HaloTag® ligands and HaloTag® conjugates. Compounds 12-15 were prepared as stock solutions in DMSO and diluted such that the final DMSO concentration did not exceed 1% v/v. All measurements were taken at ambient temperature (22 ±2° C.). Absorption spectra were recorded on a Cary Model 100 spectrometer (Agilent) using 1-cm path length 1.0-mL quartz microcuvettes from Starna Cells. Fluorescence spectra were recorded on a Cary Eclipse fluorometer (Varian) using 1-cm path length 3.5-mL quartz cuvettes (Starna Cells). As before,^{13, 23, 32, 45} HaloTag® protein was used as a 100 μM solution in 1×TBS. HaloTag® ligands 12-15 (5 μM) were dissolved in 10 mM HEPES, pH 7.3, containing 0.1 mg/mL CHAPS. An aliquot of HaloTag® protein (1.5 equiv) was added, and the resulting mixture was incubated until a consistent absorbance signal was observed (60-120 min). To measure the fold-increase of absorbance upon HaloTag® binding, a “no HaloTag®” control experiment was performed where an equivalent volume of TBS blank was added in place of the protein. Reported values for extinction coefficient (ε) are averages of at least two measurements.

Determination of K_{L-Z}. The lactone-zwitterion equilibrium constant (K_{L-Z}) was calculated as described previously^{13, 23, 32, 45} using equation 1:

$${K}_{L - Z} = \frac{\frac{{ε}_{d w}}{{ε}_{max}}}{( 1 - \frac{{ε}_{d w}}{{ε}_{max}} )} \qquad ( 1 )$$

where ε_{dw} is the extinction coefficient of the dyes in a 1:1 (v/v) dioxane:water solvent mixture containing 0.01% (v/v) triethylamine; this dioxane water mixture was chosen to give a large range of K_{L-Z} values,²³ and the triethylamine additive ensures the rhodamines are in the net neutral form. The εₘₐₓ is the maximal extinction coefficient, measured in 0.1% (v/v) trifluoroacetic acid in 2,2,2-trifluoroethanol (TFE).

Quantum yield determination. All reported absolute fluorescence quantum yield values (Φ_{f}) were measured in the laboratory under identical conditions using a Quantaurus-QY spectrometer (model C11374, Hamamatsu). This instrument uses an integrating sphere to determine photons absorbed and emitted by a sample. Measurements were performed using dilute samples (absorbance<0.1), and self-absorption corrections were performed using the instrument software.⁴⁶ Reported values are averages of at least two measurements.

Determination of logD_{7.4}. The log of distribution coefficients at pH 7.4 was determined in octanol-phosphate-buffered saline (PBS) using the miniaturized shake flask setup described previously.^{36, 47} Briefly, 150 mL each of octanol and PBS pH 7.4 were stirred vigorously for 12-16 h. The layers were allowed to stand for at least 24 h for phase separation. The separated layers were collected and served as PBS saturated with octanol (PBS*) and octanol saturated with PBS (octanol*). Stock solutions (1 mM or 5 mM) of ligands were prepared in DMSO and diluted in PBS* to obtain 1 μM or 10 μM (3-5 mL) of the standard (std). This standard was used to prepare appropriate dilutions using octanol*, in triplicate, in 2-mL glass vials. The leftover standard was used to determine the standard concentration (C_{std}). The vials were vortexed for 1 min and shaken horizontally at 700 rpm for 2 h. The vials were then shaken upright at 150 rpm for 2 h to allow the phases to separate. The octanol* layer was pipetted out, and the PBS* layer was used for further analyses. The concentrations of ligands in the standard and PBS* (C_{PBS*}) were determined via fluorescence measurements of 200 μL of PBS* or std in 96-well plates on the Cytation 5 Multimode Plate Reader (BioTek). For Si-rhodamine containing ligands, 2-20 μL of trifluoroacetic acid was added to each well of the 96-well plate prior to the fluorescence measurements. Reported values are averages of at least two measurements. The logD_{7.4} was obtained as described previously using equation 2:

$$log {D}_{7.4} = log ( ( \frac{{C}_{s t d}}{{C}_{P B S *}} \times r - 1 ) \frac{{V}_{P B S *}}{{V}_{octanol *}} ) \qquad ( 2 )$$

where C_{std} is the standard concentration in PBS, C_{PBS*} is the concentration in PBS after partitioning, r is the dilution factor of the standard solution, V_{PBS*} is the volume of PBS used in the partitioning, and V_{octanol*} is the volume of octanol used in the partitioning.

Plasmid construction. HaloTag®, amplified from the pHTC HaloTag® CMV-neo Vector (Promega; G7711) and monomeric-superfolder green fluorescent protein (msGFP; lab stock), was fused to the mitochondrial targeting signal from OMP25,²⁸ interspacing flexible GS(GSS)₄ linkers between each domain by PCR Splicing by Overlap Extension (SOE). This construct was subcloned into hSynapsin promoter bearing a copy of FUGW.²⁹ FUGW was a gift from David Baltimore (Addgene plasmid #14883; RRID: Addgene_14883).⁴⁸ This construct was designated as pF(UG) hSyn HaloTag®-TEV-4×GS-msGFP-mito.

HEK293T cell culture. HEK293T cells (ATCC) were passaged before 80% confluency by trypsinization (Corning; 25-053-CI) and trituration. The cell suspension was then plated on glass coverslips (Warner instruments; 64-0734, CS-18R17) coated with poly-D-lysine (Thermofisher; ICN10269491) and cultured in Dulbecco's Modified Eagle Medium (DMEM; ThermoFisher; 11965-118) supplemented with 10% v/v fetal bovine serum (FBS, Atlanta Biological) and penicillin/streptomycin (pen/strep; ThermoFisher; MT-30-001-CI) at 37° C. in a humidified 5% v/v CO₂ environment following ATCC guidelines. These HEK293T cells were tested for mycoplasma contamination using the Universal Mycoplasma Detection Kit (ATCC; 30-1012K) and validated using Short Tandem Repeat profiling by ATCC (ATCC; 135-XV) within the previous year. HEK293T cells were transfected with pF(UG) hSyn HaloTag®-TEV-4×GS-msGFP-mito using a standard calcium phosphate protocol.⁴⁹ This plasmid is optimized for lentivirus-mediated expression, but robust expression in HEK293T cells was obtained.

HEK293T labeling and fluorescence microscopy. One day after HEK293T cell transfection, the GFP fluorescence signal was confirmed, and HaloTag® ligand labeling experiments were conducted. For pulse-chase experiments, HEK293T cells were incubated with biotin-HaloTag® ligand (6; #G8281, Promega) at either 100 nM or 10 μM for 1 hour at 37° C. and chased with 100 nM of JANELIA FLUOR® (JF)₅₄₉-HaloTag® ligand (2_{HTL}). A commercial biotin-HaloTag® ligand with a longer polyethylene glycol (PEG) linker was also evaluated (#G8591, Promega) and it was confirmed that this is not cell-permeant, as indicated in the product information⁵⁰ and from a previous publication.³¹ For labeling with biotin-JF-HaloTag® ligands 12-15, cells were incubated with 100 nM ligand for 1 h at 37° C. For imaging, the coverslips containing HEK293T cells were fixed with 4% paraformaldehyde in PBS at 37° C. for 10 min, washed with PBS (3×), and mounted on glass slides with ProLong Glass Antifade Mountant with NucBlue™ Stain (ThermoFisher; P36981). These cells were imaged using a Zeiss LSM 880 with Airyscan and a plan-apochromatic 63×/1.40 oil objective in Fast Airyscan mode. The same image acquisition settings were used for each trial and condition. Images were then processed with automatic Airyscan deconvolution settings. For quantitation of labeling intensity, ROIs were drawn surrounding individual HEK cells, and the average fluorescence ratio (JF ligand/msGFP) from multiple cells in each condition was measured; no background subtraction was necessary. For pulse-chase experiments, a substantially reduced JF₅₄₉/msGFP ratio was observed only after labeling with 10 μM of 6 (FIG. 2C), demonstrating that μM concentrations are required to label intracellular HaloTag® proteins in live cells with 6. Q-Q plot analysis showed a normal distribution (FIG. 2D).

U2OS cell culture, labeling, and fluorescence microscopy. U2OS cells (ATCC) were cultured in Dulbecco's modified Eagle medium (DMEM, phenol red-free; Life Technologies) supplemented with 10% (v/v) fetal bovine serum (FBS, Life Technologies), 1 mM GlutaMAX (Life Technologies) and maintained at 37° C. in a humidified 5% (v/v) CO₂ environment. These cell lines undergo regular mycoplasma testing by the Janelia Cell Culture Facility. U2OS cells stably expressing an integrated HaloTag®-histone H2B fusion protein (U2OS.H2B.HaloTag®) were used for nuclear imaging and imaged 18-24 h post-plating. For other subcellular targets, U2OS cells were transiently transfected using nucleofection (Lonza) with plasmids constitutively expressing the following fusion proteins: a C-terminal transmembrane anchoring domain from platelet-derived growth factor receptor (PDGFR) fused to the HaloTag® protein (HaloTag®-PDGFR; for extracellular display); a HaloTag®-TOMM20 fusion protein (outer mitochondrial membrane; Addgene plasmid #123284; RRID: Addgene_123284); or HaloTag®-Sec61β fusion protein (endoplasmic reticulum membrane; Addgene plasmid #123285; RRID: Addgene_123285). The transiently transfected cells were imaged 18-24 h post-transfection. The stable and transiently transfected U2OS cells were incubated with 100 nM biotin-JF₅₄₉-HaloTag® ligand (12), biotin-JF₆₀₈-HaloTag® ligand (13), biotin-JF₆₄₆-HaloTag® ligand (14), or biotin-JF₆₃₅-HaloTag® ligand (15) for 1 h at 37° C., washed 3× with dye-free media, then fixed with 4% paraformaldehyde in 0.1 M phosphate buffer for 15 min at 37° C. Fixed cells were then washed 3× in 1×PBS and incubated with Hoechst 33342 (5 μg/mL) for 15 min at 22° C. as a nuclear counterstain. Airyscan imaging was performed on a Zeiss LSM 980 with Airyscan 2 confocal microscope using a Plan APO 63×/1.4 oil DIC M27 objective. The same acquisition settings were used for all constructs labeled with either 12, 13, 14, or 15. These single plane images were bulk processed in ZEN Blue (Zeiss) with automatic Airyscan settings.

Dye loading kinetics. Live U2OS.H2B.HaloTag® stable cells were labeled over a time course of 0-4 h with 200 nM of biotin-JF₅₄₉-HaloTag® ligand (12), biotin-JF₆₀₈-HaloTag® ligand (13), biotin-JF₆₄₆-HaloTag® ligand (14), or biotin-JF₆₃₅-HaloTag® ligand (15) at 37° C. Cells were then washed 3× with dye-free media, fixed with 4% paraformaldehyde in 0.1 M phosphate buffer, pH 7.4 for 15 min at 37° C. Confocal imaging was performed on a Leica SP8 with an HC PL APO CS2 20×/0.75 immersion objective using the tunable white light laser (WLL) to excite dyes at their λ_{abs} in constant power mode. Fluorescence was quantified as the average integrated density of background corrected nuclear signals from confocal image stack projections analyzed in FIJI; n=100 nuclear signals per compound.

Affinity capture and in-gel fluorescence experiments. Affinity capture pulldown isolation experiments of biotin-labeled HaloTag® fusion proteins were performed on ice-cold isolation buffer (IB) consisting of KCl (0.18 M), ethylene glycol-bis(β-aminoethyl ether)-N,N,N′,N′-tetraacetic acid (EGTA; 1 mM), 3-(N-morpholino)propanesulfonic acid (MOPS; 5 mM); the pH was adjusted to pH=7.35 using KOH(aq).⁵¹ HEK293T cells were scraped with 500 μL IB and homogenized using 12 strokes with a 27G syringe. The resulting homogenate was spun at 4° C. at 1000 g for 3 min to pellet large cellular debris. 50 μL of streptavidin-coated microbeads (Miltenyi, 130-048-101) were added to the supernatant and incubated for 30 min at 4° C. with slow rotation. LS columns (Miltenyi, 130-042-401) were prepared by first washing with 3 mL IB consisting of 2.5% bovine serum albumin (BSA; Jackson ImmunoResearch, 001-000-162) and then with plain IB (3×). Prior to supernatant-microbead mixture being loaded onto LS columns, 5% volume was taken for downstream total fraction calculations (i.e., the “total/20” sample). Columns and samples were kept at 4° C. (cold room) while samples were loaded onto columns and washed with IB (3×); this fraction was collected as designated as “wash”. After the washing step, the column was removed from the QuadroMACs magnet holder (Miltenyi, 130-092-857) and eluted with gentle plunge action into an Eppendorf tube; this was labeled as “eluate”. From here, there were three fractions for each condition: total/20, wash (unbound), and eluate (bound). Since mitochondria were being isolated, these samples were spun at 4° C. and 20,000 g to pellet mitochondria. These samples were then solubilized using the lysis buffer containing 1×PBS, 2% SDS, 1% Triton x-100, 10 mM EDTA, and protease inhibitors (2 mM PMSF, aprotinin, leupeptin, and pepstatin A). Samples were then incubated at 72° C. for 3-4 min after the addition of sample buffer (SB) (DTT, glycerol, and bromophenol blue). For protein detection, 10-20 μL of the sample was subjected to sodium dodecyl sulfate-polyacrylamide gel electrophoresis (SDS-PAGE), using 13% gels. The affinity capture pulldown efficiency was monitored using in-gel fluorescence. The msGFP signal from HaloTag®-TEV-4×GS-msGFP-mito after cell lysis and SDS-PAGE was analyzed, in-gel, using a BioRad Chemidoc MP imager (BioRad) through GFP fluorescence excitation and emission filters. Data were quantified by densitometry using Fiji.⁵² Capture efficiency was calculated using equation 3:

$$Capture efficiency = \frac{{{I}_{eluate}}_{}}{{I}_{total / 20} \times 20} \qquad ( 3 )$$

where Iₑₗᵤₐₜₑ is the in-gel fluorescence intensity of the bead-bound fraction and I_{total/20} is the in-gel fluorescence intensity of the 5% of crude sample reserved for concentration control (i.e., the “total/20” sample).

### General Synthetic Organic Chemistry Methods

Commercial reagents were the highest quality available and used as received. Solvents for reactions were of anhydrous grade, purchased in septum-sealed bottles, and stored under an inert atmosphere. Reactions were conducted in round-bottomed flasks or septum-sealed crimp-top microwave reaction vials (Biotage) containing Teflon-coated magnetic stir bars. All reactions were conducted under an inert atmosphere of Ar(g) and protected from light using Al foil unless otherwise noted. Heating of reaction mixtures was achieved through aluminum blocks on top of a stirring hotplate equipped with an electronic contact thermometer. Reactions were monitored either by thin layer chromatography (TLC) on precoated TLC glass plates (silica gel 60 F254, 250 μm thickness) or by tandem liquid chromatography-mass spectrometry (LC-MS; Shimadzu LCMS 2020, Phenomenex Kinetex 30×2.1 mm 2.6 μm C18 column, 1-10 μL injection, 5-98% CH₃CN/H₂O linear gradient with constant 0.1% v/v HCO₂H, 6 min run, 1 mL/min flowrate, ESI, positive ion mode). TLC plates were visualized either by UV illumination or by developing the TLC with ceric ammonium molybdate or KMnO₄.

Reaction products were purified either by flash chromatography on Biotage Isolera automated purification system using prepacked silica gel columns and/or by preparative high-pressure liquid chromatography (HPLC; Agilent 1200, Phenomenex Gemini-NX 150×30 mm 10 μm C18 110 Å column, 42 mL/min flowrate) under the indicated solvent gradient conditions. Analytical HPLC analyses were performed on a LC-MS system (Agilent 1200, Phenomenex Gemini-NX 150×4.6 mm 5 μm C18 110 Å column, 1 mL/min flowrate) or an analytical HPLC (Shimadzu UFLC, Phenomenex Gemini-NX 150×4.6 mm 5 μm C18 110 Å column, 1 mL/min flowrate) under the indicated conditions. All the HPLC systems are fitted with a diode array detector. High-resolution mass spectrometry was obtained from the High Resolution Mass Spectrometry Facility at the University of Iowa. NMR spectra were recorded on Bruker Avance 400 MHz spectrometer and processed through MestReNova. Deuterated solvents were used as purchased. ¹H and ¹³C chemical shifts (δ) were referenced to TMS or residual solvent peaks. ¹⁹F chemical shifts were referenced to CFCl₃. Data for ¹H NMR spectra are reported as follows: chemical shift (δ ppm), multiplicity (s=singlet, d=doublet, t=triplet, q=quartet, p=pentet (quintet), dd=doublet of doublets, dt=doublet of triplets, m=multiplet, br=broad signal), coupling constant (Hz), and integration. Data for ¹³C NMR spectra are reported by chemical shift (δ ppm) with hydrogen multiplicity (C, CH, CH₂, CH₃) information obtained from DEPT spectra.

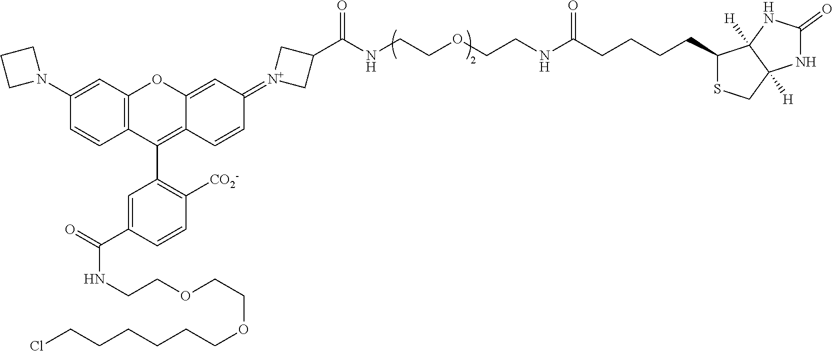

Biotin-JF₅₄₉-HaloTag® ligand (12). The TFA salt of 3″-carboxy-JF₅₄₉ -HaloTag® ligand¹⁴ (7; 10.0 mg, 8.5 μmol, 1 equiv) was dissolved in anhydrous DMF (2 mL). To this solution were added Et₃N (12.0 μL, 85 μmol, 10 equiv), DSC (5.5 mg, 21 μmol, 2.5 equiv), and a catalytic amount of DMAP (˜0.05 mg). The reaction mixture was stirred for 90 min at ambient temperature, after which biotin-PEG₂-NH₂ (11, 32.0 mg, 85 μmol, 10 equiv) was added. The reaction mixture was further stirred for 16 h at ambient temperature. The solvent was removed under reduced pressure, and the product was purified by preparative HPLC using a 5-95% CH₃CN/H₂O linear gradient with constant 0.1% v/v trifluoroacetic acid (TFA). Product-containing fractions were combined and lyophilized to obtain 12 as a red solid (TFA salt, 4.5 mg, 51%). ¹H NMR (CD₃OD, 400 MHz) δ 8.39 (d, J=8.2 Hz, 1H), 8.21 (dd, J=8.2, 1.8 Hz, 1H), 7.81 (d, J=1.8 Hz, 1H), 7.08 (d, J=9.1 Hz, 2H), 6.63 (ddd, J=9.2, 5.3, 2.1 Hz, 2H), 6.59 (d, J=2.1 Hz, 1H), 6.53 (d, J=2.1 Hz, 1H), 4.48-4.39 (m, 3H), 4.38-4.23 (m, 7H), 3.74-3.51 (m, 19H), 3.48-3.41 (m, 4H), 3.36 (t, J=5.5 Hz, 2H), 3.22-3.14 (m, 1H), 2.89 (dd, J=12.8, 5.0 Hz, 1H), 2.66 (dd, J=12.8, 3.0 Hz, 1H), 2.56 (p, J=7.4 Hz, 2H), 2.21 (t, J=7.4 Hz, 2H), 1.77-1.55 (m, 6H), 1.53-1.47 (m, 2H), 1.45-1.30 (m, 6H). ¹³C NMR (CD₃OD, 101 MHz) δ 176.12 (C), 173.82 (C), 167.92 (C), 167.34 (C), 166.03 (C), 160.59 (C), 158.93 (C), 158.63 (C), 158.18 (C), 157.68 (C), 139.40 (C), 135.58 (C), 134.75 (C), 132.84 (CH), 132.44 (CH), 132.33 (CH), 130.37 (CH), 130.05 (CH), 115.15 (C), 114.96 (C), 113.94 (CH), 113.49 (CH), 95.61 (CH), 95.18 (CH), 72.14 (CH₂), 71.31 (CH₂), 71.24 (CH₂), 71.14 (CH₂), 70.61 (CH₂), 70.46 (CH₂), 70.35 (CH₂), 63.33 (CH), 61.61 (CH), 56.97 (CH), 55.32 (CH₂), 52.98 (CH₂), 45.75 (CH₂), 41.21 (CH₂), 41.06 (CH₂), 40.63 (CH₂), 40.28 (CH₂), 36.74 (CH₂), 34.62 (CH), 33.72 (CH₂), 30.46 (CH₂), 29.76 (CH₂), 29.50 (CH₂), 27.69 (CH₂), 26.84 (CH₂), 26.44 (CH₂), 16.81 (CH₂). ¹⁹F NMR (CD₃OD, 376 MHz) δ −75.35. Analytical HPLC: t_{R}=11.2 min, 98.0% purity (10-95% MeCN/H₂O linear gradient over 20 min with constant 0.1% v/v TFA, 1 mL/min flow rate, detection at 550 nm). HRMS (ESI) calculated for C₅₄H₇₁N₇O₁₁SCl [M+H]⁺=1060.4615, found 1060.4623.

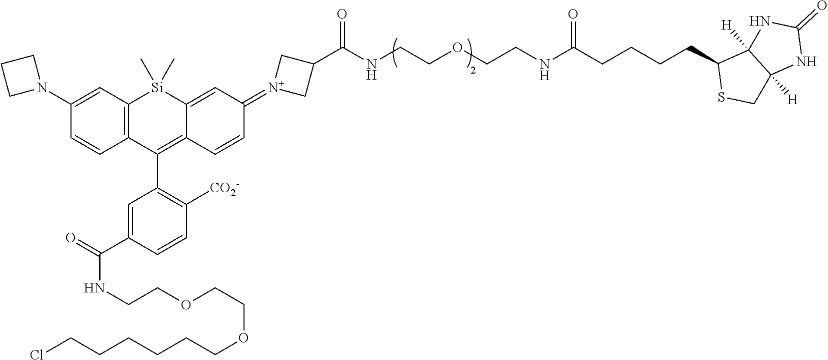

Biotin-JF₆₄₆-HaloTag® ligand (14). The TFA salt of 3″-carboxy-JF₆₄₆ -HaloTag® ligand¹⁴ (9; 57.0 mg, 66 μmol, 1 equiv) was dissolved in DMF (2 mL). To this solution were added Et₃N (96.0 μL, 661 μmol, 10 equiv), DSC (42.3 mg, 165.2 μmol, 2.5 equiv), and a catalytic amount of DMAP (˜0.05 mg). The reaction mixture was stirred for 90 min at ambient temperature, after which biotin-PEG₂-NH₂ (11, 99.0 mg, 264 μmol, 4 equiv) was added. The reaction mixture was stirred overnight at ambient temperature. The solvent was removed under reduced pressure, and the product was purified by preparative HPLC using a 30-50% CH₃CN/H₂O linear gradient with a constant 0.1% v/v TFA. Product-containing fractions were combined and lyophilized to obtain 14 as a blue solid (TFA salt, 43 mg, 58.1%). ¹H NMR (CD₃OD, 400 MHz) δ 8.24 (d, J=8.2 Hz, 1H), 8.10 (dd, J=8.2, 1.7 Hz, 1H), 7.69 (d, J=1.6 Hz, 1H), 6.92 (d, J=2.5 Hz, 2H), 6.88 (dd, J=9.2, 7.9 Hz, 2H), 6.35 (dd, J=9.2, 2.6 Hz, 2H), 4.47-4.42 (m, 1H), 4.41-4.18 (m, 9H), 3.67-3.49 (m, 19H), 3.45-3.40 (m, 4H), 3.35 (t, J=5.5 Hz, 2H), 3.20-3.12 (m, 1H), 2.88 (dd, J=12.8, 5.0 Hz, 1H), 2.66 (d, J=12.7 Hz, 1H), 2.51 (p, J=7.6 Hz, 2H), 2.20 (t, J=7.4 Hz, 2H), 1.76-1.55 (m, 6H), 1.53-1.47 (m, 2H), 1.46-1.29 (m, 6H), 0.60 (s, 3H), 0.55 (s, 3H). ¹³C NMR (CD₃OD, 400 MHz) δ 176.11 (C), 174.04 (C), 168.44 (C), 168.22 (C), 166.04 (C), 154.06 (C), 153.57 (C), 139.38 (C), 133.58 (C), 131.20 (CH), 130.41 (CH), 130.25 (CH), 129.00 (CH), 119.57 (CH), 118.98 (CH), 113.39 (CH), 113.08 (CH), 72.12 (CH₂), 71.30 (CH₂), 71.20 (CH₂), 71.13 (CH₂), 70.61 (CH₂), 70.47 (CH₂), 70.36 (CH₂), 63.34 (CH), 61.60 (CH), 56.97 (CH), 55.39 (CH₂), 53.32 (CH₂), 45.74 (CH₂), 41.14 (CH₂), 41.05 (CH₂), 40.59 (CH₂), 40.27 (CH₂), 36.74 (CH₂), 34.90 (CH), 33.71 (CH₂), 30.44 (CH₂), 29.75 (CH₂), 29.49 (CH₂), 27.68 (CH₂), 26.83 (CH₂), 26.42 (CH₂), 17.04 (CH₂), −0.74 (CH₃), −1.66 (CH₃). Five aromatic quaternary carbons were not observed. ¹⁹F NMR (CD₃OD, 376 MHz) δ −75.35. Analytical HPLC: t_{R}=11.9 min, 97.0% purity (10-95% MeCN/H₂O linear gradient over 20 min with constant 0.1% v/v TFA, 1 mL/min flow rate, detection at 254 nm). HRMS (ESI) calculated for C₅₆H₇₆N₇O₁₀SClSiNa [M+Na]⁺=1124.4724, found 1124.4730.

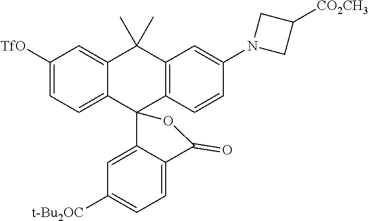

methyl 1-(6′-(tert-butoxycarbonyl)-10,10-dimethyl-3′-oxo-3-(((trifluoromethyl)sulfonyl)oxy)-3′H, 10H-spiro[anthracene-9,1′-isobenzofuran]-6-yl)azetidine-3-carboxylate (S3). An oven-dried microwave reaction vial was charged with 6-tert-butoxycarbonyl carbofluorescein ditriflate² (S1, 722.6 mg, 1 mmol, 1 equiv), Pd₂dba₃ (46 mg, 50 μmol, 0.05 equiv), XPhos (72 mg, 150 μmol, 0.15 equiv), methyl azetidine-3-carboxylate hydrochloride (S2, 182 mg, 1.2 mmol, 1.2 equiv), and Cs₂CO₃ (912 mg, 2.8 mmol, 2.8 equiv). The vial was sealed and backfilled with Ar(g) (3×), after which anhydrous dioxane (10 mL) was added. The resulting mixture was stirred at 80° C. for 3 h. The reaction was cooled and filtered through a pad of celite using EtOAc. The filtrate was concentrated under reduced pressure. Purification by SiO₂ gel chromatography (50 g SiO₂ column, 0-50% EtOAc/hexanes, linear gradient) provided monoazetidine compound S3 as a light green foamy solid (220 mg) and unreacted starting material (168 mg). LC-MS (ESI): calculated for C₃₄H₃₃F₃NO₉S [M+H]⁺=688.18, found 688.18. This material was used immediately in the subsequent synthetic step.

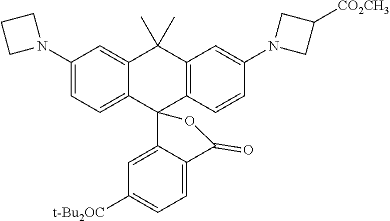

methyl 1-(3-(azetidin-1-yl)-6′-(tert-butoxycarbonyl)-10, 10-dimethyl-3′-oxo-3′H, 10H-spiro[anthracene-9,1′-isobenzofuran]-6-yl)azetidine-3-carboxylate (i.e., 3″-methoxycarbonyl-6-tert-butoxycarbonyl-JF₆₀₈; S5). An oven-dried microwave reaction vial was charged with S3 (220 mg, 320 μmol, 1 equiv), Pd₂dba₃ (29 mg, 32 μmol, 0.1 equiv), XPhos (46 mg, 96 μmol, 0.3 equiv), azetidine (S4, 108 μL, 91 mg, 1.6 mmol, 5 equiv), and Cs₂CO₃ (292 mg, 896 μmol, 2.8 equiv). The vial was sealed and backfilled with Ar(g) (3×), after which anhydrous dioxane (10 mL) was added. The resulting mixture was stirred at 100° C. for 4 h, then cooled to room temperature and filtered through a pad of celite using EtOAc. The filtrate was concentrated under reduced pressure. Purification by SiO₂ gel chromatography (50 g SiO₂ column, 0-100% EtOAc/hexanes, linear gradient) provided S5 as a green-blue solid (153 mg, 33.4% over two steps). ¹H NMR (CDCl₃, 400 MHz) δ 8.15 (dd, J=8.0, 1.3 Hz, 1H), 8.01 (d, J=8.0 Hz, 1H), 7.61 (t, J=1.0 Hz, 1H), 6.63-6.51 (m, 4H), 6.23 (ddd, J=8.8, 5.1, 2.3 Hz, 2H), 4.15-4.03 (m, 4H), 3.92 (t, J=7.2 Hz, 4H), 3.75 (s, 3H), 3.58 (tt, J=8.6, 6.1 Hz, 1H), 2.38 (p, J=7.2 Hz, 2H), 1.83 (s, 3H), 1.73 (s, 3H), 1.53 (s, 9H). ¹³C NMR (101 MHz, CDCl₃) δ 173.21 (C), 170.08 (C), 164.51 (C), 155.51 (C), 152.37 (C), 151.63 (C), 146.92 (C), 146.67 (C), 137.85 (C), 130.17 (CH), 130.13 (C), 128.99 (CH), 128.91 (CH), 125.05 (CH), 124.87 (CH), 120.85 (C), 119.77 (C), 110.77 (CH), 110.62 (CH), 108.43 (CH), 108.05 (CH), 82.36 (C), 54.55 (CH₂), 54.52 (CH₂), 52.38 (CH₃), 52.36 (CH₂), 38.49 (C), 35.43 (CH₃), 33.54 (CH₃), 32.81 (CH), 28.16 (CH₃), 16.94 (CH₂). HRMS (ESI) calculated for C₃₆H₃₉N₂O₆ [M+H]⁺=595.2803, found 595.2804.

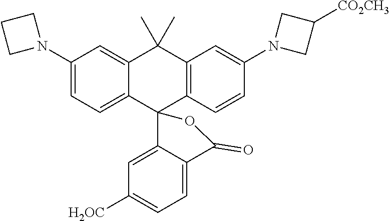

6-carboxy-3″-methoxycarbonyl-JF₆₀₈ (S6). Compound S5 (32 mg, 54 μmol) was dissolved in CH₂Cl₂ (2 mL). To this solution was added TFA (0.4 mL), and the resulting blue solution was stirred at ambient temperature for 7 h. The reaction was concentrated under reduced pressure, yielding S6 as a blue solid. HRMS (ESI): calculated for C₃₂H₃₁N₂O₆ [M+H]⁺=539.2177, found 539.2182. This material was used immediately in the subsequent synthetic step.

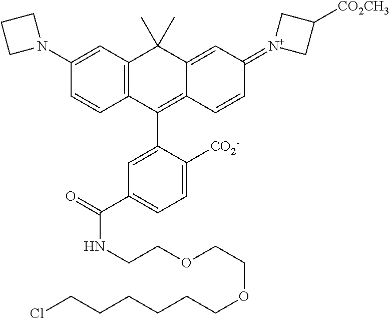

3″-methoxycarbonyl-JF₆₀₈ -HaloTag® ligand (S8). Compound S6 was dissolved in DMF (2 mL). To this solution were added DIEA (96 μL, 538 μmol, 10 equiv) and TSTU (24 mg, 81 μmol, 1.5 equiv). The reaction mixture was stirred for 10 min at ambient temperature, after which HaloTag®(O₂)—NH₂ (S7, 21 mg, 81 μmol, 1.5 equiv) was added. The reaction mixture was stirred for 16 h at ambient temperature. The solvent was removed under reduced pressure to yield S8 as a blue solid. LC-MS (ESI) calculated for C₄₂H₅₁ClN₃O₇ [M+H]⁺=744.34, found 744.40. This material was used immediately in the subsequent synthetic step.

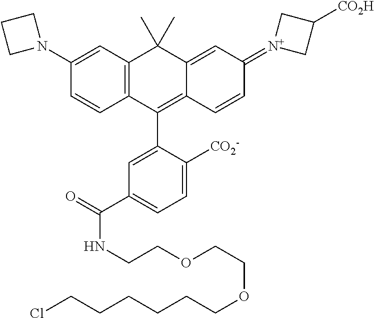

3″-carboxy-JF₆₀₈ -HaloTag® ligand (10). NaOH (1 M, 108 μL) was added to a solution of S8 in CH₃OH (4 mL). The resulting solution was stirred for 14 h. The reaction mixture was acidified to pH˜1 using 1M HCl. This crude product was purified by preparative HPLC using a 25-75% CH₃CN/H₂O linear gradient with a constant 0.1% v/v TFA. Product-containing fractions were combined and lyophilized to obtain 10 as a blue solid (TFA salt, 19 mg, 41.8% over three steps). ¹H NMR (CDCl₃, 400 MHz) δ 8.33 (d, J=8.3 Hz, 1H), 8.15 (dd, J=8.2, 1.8 Hz, 1H), 7.73 (d, J=1.8 Hz, 1H), 6.94 (t, J=9.0 Hz, 2H), 6.87 (d, J=2.3 Hz, 1H), 6.84 (d, J=2.2 Hz, 1H), 6.41 (t, J=2.1 Hz, 1H), 6.39 (t, J=2.1 Hz, 1H), 4.47 (t, J=9.5 Hz, 2H), 4.41-4.30 (m, 6H), 3.72-3.67 (m, 1H), 3.66-3.59 (m, 5H), 3.58-3.55 (m, 3H), 3.52 (t, J=6.6 Hz, 2H), 3.43 (t, J=6.5 Hz, 2H), 2.55 (p, J=7.6 Hz, 2H), 1.82 (s, 3H), 1.75-1.68 (m, 5H), 1.54-1.46 (m, 2H), 1.44-1.37 (m, 2H), 1.37-1.30 (m, 2H). ¹³C NMR (101 MHz, CDCl₃) δ 175.37 (C), 168.10 (C), 167.62 (C), 158.20 (C), 157.25 (C), 156.98 (C), 156.31 (C), 140.23 (C), 139.03 (C), 137.85 (CH), 137.16 (CH), 134.71 (C), 132.24 (CH), 129.97 (CH), 129.35 (CH), 122.05 (C), 121.98 (C), 112.26 (CH), 111.82 (CH), 110.00 (CH), 109.79 (CH), 72.12 (CH₂), 71.20 (CH₂), 71.13 (CH₂), 70.36 (CH₂), 55.25 (CH₂), 53.06 (CH₂), 45.73 (CH₂), 42.70 (C), 41.15 (CH₂), 35.36 (CH₃), 33.91 (CH), 33.71 (CH₂), 32.27 (CH₃), 30.44 (CH₂), 27.68 (CH₂), 26.43 (CH₂), 16.83 (CH₂). HRMS (ESI) calculated for C₄₁H₄₉ClN₃O₇ [M+H]⁺=730.3254, found 730.3255.

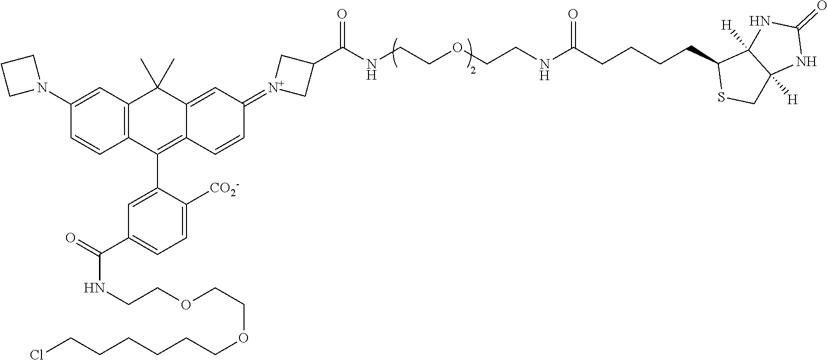

Biotin-JF₆₀₈-HaloTag® ligand (13). The TFA salt of 10 (19 mg, 22.5 μmol, 1 equiv) was dissolved in DMF (3 mL). To this solution were added DIEA (40 μL, 225 μmol, 10 equiv), biotin-PEG₂-NH₂ (9, 9 mg, 33.7 μmol, 1.5 equiv), and HATU (12 mg, 33.8 μmol, 1.5 equiv). The reaction mixture was stirred for 18 h at ambient temperature. The solvent was removed under reduced pressure, and the product was purified by preparative HPLC using a 30-60% CH₃CN/H₂O linear gradient with a constant 0.1% v/v TFA. Product-containing fractions were combined and lyophilized to obtain 13 as a blue solid (TFA salt, 19.8 mg, 73.3%). ¹H NMR (CD₃OD, 400 MHz) δ 8.34 (d, J=8.3 Hz, 1H), 8.15 (dd, J=8.3, 1.8 Hz, 1H), 7.75 (d, J=1.8 Hz, 1H), 6.97 (d, J=6.8 Hz, 1H), 6.95 (d, J=6.8 Hz, 1H), 6.87 (d, J=2.2 Hz, 1H), 6.84 (d, J=2.2 Hz, 1H), 6.42 (t, J=2.4 Hz, 1H), 6.39 (t, J=2.5 Hz, 1H), 4.50-4.31 (m, 9H), 4.27 (dd, J=7.9, 4.4 Hz, 1H), 3.67-3.51 (m, 19H), 3.43 (t, J=6.4 Hz, 4H), 3.36 (t, J=5.5 Hz, 2H), 3.18 (dt, J=8.9, 5.3 Hz, 1H), 2.89 (ddd, J=12.8, 5.0, 1.0 Hz, 1H), 2.66 (dt, J=12.8, 1.2 Hz, 1H), 2.56 (p, J=7.6 Hz, 2H), 2.21 (t, J=7.3 Hz, 2H), 1.82 (s, 3H), 1.73-1.63 (m, 6H), 1.67-1.56 (m, 3H), 1.51-1.47 (m, 2H), 1.46-1.31 (m, 6H). ¹³C NMR (CD₃OD, 101 MHz) δ 176.13 (C), 173.93 (C), 168.08 (C), 167.44 (C), 166.05 (C), 158.51 (C), 157.65 (C), 157.03 (C), 156.37 (C), 139.48 (C), 138.91 (C), 138.11 (CH), 137.51 (CH), 134.86 (C), 132.53 (CH), 130.26 (CH), 129.33 (CH), 122.03 (C), 121.99 (C), 112.21 (CH), 111.83 (CH), 109.98 (CH), 109.78 (CH), 72.12 (CH₂), 71.30 (CH₂), 71.20 (CH₂), 71.13 (CH₂), 70.60 (CH₂), 70.45 (CH₂), 70.36 (CH₂), 63.33 (CH), 61.60 (CH), 56.99 (CH), 55.24 (CH₂), 53.02 (CH₂), 45.75 (CH₂), 42.83 (C), 41.15 (CH₂), 41.06 (CH₂), 40.61 (CH₂), 40.27 (CH₂), 36.73 (CH₂), 35.40 (CH₃), 34.63 (CH₃), 33.71 (CH₂), 32.23 (CH), 30.45 (CH₂), 29.77 (CH₂), 29.50 (CH₂), 27.68 (CH₂), 26.84 (CH₂), 26.43 (CH₂), 16.80 (CH₂). ¹⁹F NMR (CD₃OD, 376 MHz) δ −75.45. HRMS (ESI) calculated for C₅₇H₇₇N₇O₁₀SCl [M+H]⁺=1086.5136, found 1086.5136.

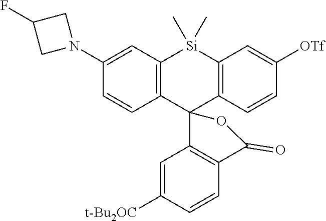

tert-butyl 3-(3-fluoroazetidin-1-yl)-5,5-dimethyl-3′-oxo-7-(((trifluoromethyl)sulfonyl)oxy)-3′H,5H-spiro[dibenzo[b,e]siline-10,1′-isobenzofuran]-6′-carboxylate (S11). An oven-dried microwave reaction vial was charged with 6-tert-butoxycarbonyl-Si-fluorescein ditriflate² (S9, 370 mg, 500 μmol, 1 equiv), Pd₂dba₃ (46 mg, 50 μmol, 0.1 equiv), XantPhos (87 mg, 150 μmol, 0.3 equiv), 3-fluoroazetidine hydrochloride (S10, 62 mg, 550 μmol, 1.1 equiv), and Cs₂CO₃ (381 mg, 1.17 mmol, 2.4 equiv). The vial was sealed and backfilled with Ar(g) (3×), after which anhydrous dioxane (3 mL) was added. The resulting mixture was stirred at 80° C. for 2.5 h. The reaction was cooled and filtered through a pad of celite using CH₂Cl₂. The filtrate was concentrated under reduced pressure. Purification by SiO₂ gel chromatography (50 g SiO₂ column, 0-25% EtOAc/hexanes, linear gradient) provided monoazetidine compound S11 as a light green foamy solid (210 mg). LC-MS (ESI): calculated for C₃₁H₂₉F₄NO₇SSi [M+H]⁺=663.14, found 664.15. This material was used immediately in the subsequent synthetic step.

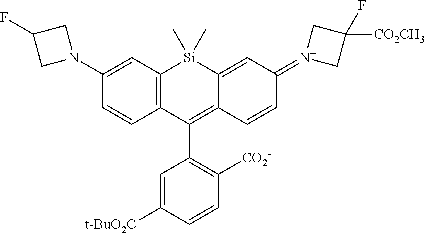

4-(tert-butoxycarbonyl)-2-(3-(3-fluoro-3-(methoxycarbonyl)azetidin-1-ium-1-ylidene)-7-(3-fluoroazetidin-1-yl)-5,5-dimethyl-3,5-dihydrodibenzo[b,e]silin-10-yl)benzoate (i.e., 3″-methoxycarbonyl-6-tert-butoxycarbonyl-JF₆₃₅; S13). An oven-dried microwave reaction vial was charged with S11 (210 mg, 316 μmol, 1 equiv), Pd₂dba₃ (29 mg, 32 μmol, 0.1 equiv), XPhos (55 mg, 95 μmol, 0.3 equiv), methyl 3-fluoroazetidine-3-carboxylate hydrochloride (S12, 161 mg, 949 μmol, 3 equiv), and Cs₂CO₃ (495 mg, 1.52 mmol, 4.8 equiv). The vial was sealed and backfilled with Ar(g) (3×), after which anhydrous dioxane (4 mL) was added. The resulting mixture was stirred at 100° C. for 4 h, then cooled to room temperature and filtered through a pad of celite using CH₂Cl₂. The filtrate was concentrated under reduced pressure. Purification by SiO₂ gel chromatography (50 g SiO₂ column, 0-40% EtOAc/hexanes, linear gradient) provided S13 as a light green solid (177 mg, 55% over two steps). ¹H NMR (CDCl₃, 400 MHz) δ 8.13 (dd, J=8.0, 1.3 Hz, 1H), 7.98 (dd, J=8.0, 0.7 Hz, 1H), 7.83 (t, J=1.0 Hz, 1H), 6.91 (dd, J=8.7, 7.2 Hz, 2H), 6.72 (dd, J=5.6, 2.7 Hz, 2H), 6.36 (ddd, J=8.7, 5.0, 2.7 Hz, 2H), 5.46-5.46 (m, 0.5H), 5.36-5.31 (m, 0.5H), 4.42-4.34 (m, 2H), 4.24-4.13 (m, 4H), 4.04-4.00 (m, 1H), 3.98-3.94 (m, 1H), 3.87 (s, 3H), 1.55 (s, 9H), 0.68 (s, 3H), 0.60 (s, 3H). ¹³C NMR (101 MHz, CDCl₃) δ 170.11 (C), 168.67 (d, ²J_{CF}=28.0 Hz, C), 164.32 (C), 155.11 (C), 149.90 (C), 149.05 (C), 137.37 (C), 136.40 (C), 136.16 (C), 133.99 (C), 133.37 (C), 130.07 (CH), 128.89 (C), 127.75 (CH), 127.73 (CH), 125.79 (CH), 125.00 (CH), 116.28 (CH), 116.23 (CH), 113.35 (CH), 113.22 (CH), 91.21 (C), 88.28 (d, ¹J_{CF}=219.9 Hz, C), 82.76 (d, ¹J_{CF}=204.9 Hz, CH), 82.43 (C), 61.22 (d, ²J_{CF}=25.1 Hz, CH₂), 59.55 (d, ²J_{CF}=23.8 Hz, CH₂), 53.25 (CH₃), 28.19 (CH₃), 0.11 (CH₃), −0.69 (CH₃). HRMS (ESI) calculated for C₃₅H₃₇F₂N₂O₆Si [M+H]⁺=647.2383, found 647.2376.

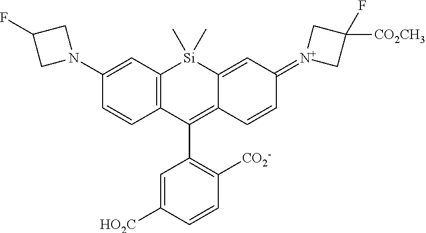

6-carboxy-3″-methoxycarbonyl-JF₆₃₅ (S14). Compound S13 (16 mg, 25 μmol, 1 equiv) was dissolved in CH₂Cl₂ (2 mL). To this solution was added TFA (0.4 mL), and the resulting blue solution was stirred at ambient temperature for 4 h. The reaction was concentrated under reduced pressure, yielding S14 as a blue solid. LC-MS (ESI): calculated for C₃₁H₂₉F₂N₂O₆Si [M]⁺=591.18, found 591.25. This material was used immediately in the subsequent synthetic step.

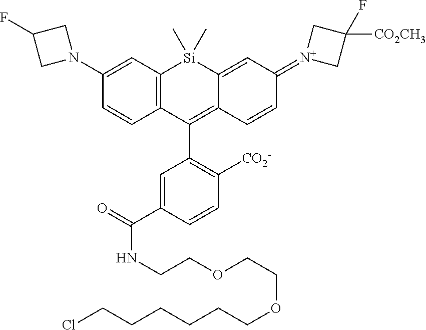

3″-methoxycarbonyl-JF₆₃₅ - HaloTag® ligand (S15). Compound S14 was dissolved in DMF (2 mL). To this solution were added N,N-Diisopropylethylamine (DIEA; 43 μL, 247 μmol, 10 equiv) and N,N,N′,N′-tetramethyl-O-(N-succinimidyl)uronium tetrafluoroborate (TSTU; 12.3 mg, 41 μmol, 1.65 equiv). The reaction mixture was stirred for 10 min at ambient temperature, after which HaloTag®(O₂)—NH₂ (S7, 8 mg, 31 μmol, 1.25 equiv) was added. The reaction mixture was stirred for 16 h at ambient temperature. The solvent was removed under reduced pressure to yield S15 as a blue solid. LC-MS (ESI) calculated for C₄₁H₄₉ClF₂N₃O₇Si [M]⁺=796.30, found 796.20. This material was used immediately in the subsequent synthetic step.

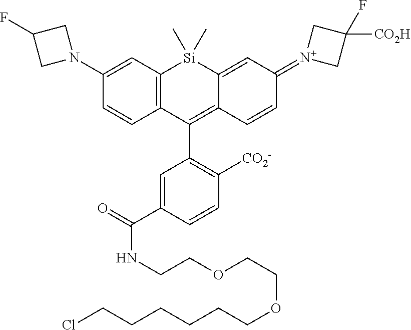

3″-carboxy-JF₆₃₅ -HaloTag® ligand (10). Compound S15 was dissolved in a mixture of THF:CH₃OH (1:1 v/v; 4 mL). An aqueous solution of NaOH (1 M, 266 μL) was added, and the resulting solution and stirred for 7 h. The reaction mixture was diluted with H₂O (2 mL) and acidified to pH˜1 using an aqueous solution of HCl (1 M). The mixture was extracted with EtOAc (4×2 mL), and the combined organics were concentrated under reduced pressure. This crude product was purified by preparative HPLC using a 5-95% CH₃CN/H₂O linear gradient with a constant 0.1% v/v TFA. Product-containing fractions were combined and lyophilized to obtain 10 as a blue solid (TFA salt, 13.6 mg, 61% over three steps). ¹H NMR (CDCl₃, 400 MHz) δ 8.02-8.0 (m, 1H), 7.92-7.90 (m, 1H), 7.72 (s, 1H), 7.20 (t, J=1.0 Hz, 1H), 6.80 (dd, J=8.7, 1.4 Hz, 2H), 6.72 (dd, J=12.6, 2.6 Hz, 2H), 6.62 (d, J=2.6 Hz, 1H), 6.31 (dd, J=8.8, 2.6 Hz, 1H), 6.26 (dd, J=8.9, 2.6 Hz, 1H), 5.52-5.49 (m, 0.5H), 5.35-5.33 (m, 0.5H), 4.39-4.24 (m, 4H), 4.18-4.01 (m, 4H), 3.64-3.62 (m, 6H), 3.58-3.55 (m, 2H), 3.49 (t, J=6.6 Hz, 2H), 3.42 (t, J=6.7 Hz, 2H), 1.75-1.67 (m, 2H), 1.44 (p, J=6.9 Hz, 2H), 1.43-1.35 (m, 2H), 1.33-1.27 (m, 2H), 0.53 (s, 3H), 0.46 (s, 3H). ¹³C NMR (101 MHz, CDCl₃) δ 169.99 (d, ²J_{CF}=28.3 Hz, C), 169.44 (C), 167.06 (C), 152.36 (C), 150.32 (C), 149.73 (C), 139.34 (C), 138.98 (C), 138.85 (C), 132.95 (C), 132.62 (C), 130.27 (C), 129.77 (C), 127.77 (CH), 127.20 (CH), 124.83 (CH), 117.29 (CH), 116.92 (CH), 113.42 (CH), 113.03 (CH), 87.73 (d, ¹J_{CF}=220.4 Hz, C), 82.49 (d, ¹J_{CF}=205.0 Hz, CH), 71.40 (CH₂), 70.22 (CH₂), 69.99 (CH₂), 69.48 (CH₂), 61.29 (d, ²J_{CF}=25.2 Hz, CH₂), 59.74 (d, ²J_{CF}=24.9 Hz, CH₂), 45.14 (CH₂), 40.35 (CH₂), 32.57 (CH₂), 29.35 (CH₂), 26.72 (CH₂), 25.41 (CH₂), 0.13 (CH₃), −1.43 (CH₃). One aromatic quaternary carbon was not observed. HRMS (ESI) calculated for C₄₀H₄₇ClF₂N₃O₇Si [M+H]⁺=782.2834, found 782.2823.

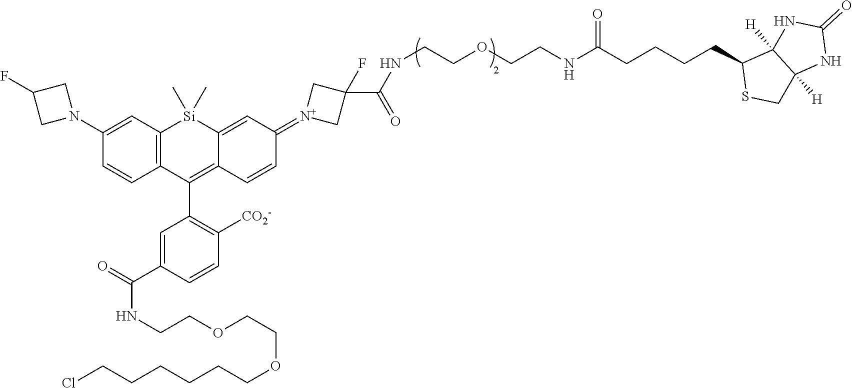

Biotin-JF₆₃₅-HaloTag® ligand (15). The TFA salt of 10 (61.9 mg, 69 μmol, 1 equiv) was dissolved in DMF (2 mL). To this solution were added DIEA (121 μL, 690 μmol, 10 equiv), N-(3-dimethylaminopropyl)-N′-ethylcarbodiimide hydrochloride (EDC.HCl; 17.2 mg, 90 μmol, 1.3 equiv), and 1-[bis(dimethylamino)methylene]-1H-1,2,3-triazolo[4,5-b]pyridinium 3-oxid hexafluorophosphate (HATU; 34.2 mg, 90 μmol, 1.3 equiv). The reaction mixture was stirred at ambient temperature for 10 min, after which biotin-PEG₂-NH₂ (11, 27.4 mg, 104 μmol, 1.5 equiv) was added. The reaction mixture was stirred for a further 16 h at ambient temperature. The solvent was removed under reduced pressure, and the product was purified by preparative HPLC using a 40-55% CH₃CN/H₂O linear gradient with a constant 0.1% v/v TFA. Product-containing fractions were combined and lyophilized to obtain 15 as a blue solid (TFA salt, 42.4 mg, 48.9%). ¹H NMR (CD₃OD, 400 MHz) δ 7.97 (q, J=8.0 Hz, 2H), 7.59 (d, J=1.6 Hz, 1H), 6.78 (dd, J=14.7, 2.6 Hz, 2H), 6.72 (dd, J=8.9, 4.7 Hz, 2H), 6.30 (ddd, J=11.9, 8.9, 2.6 Hz, 2H), 5.40 (tt, J=6.1, 3.3 Hz, 0.5H), 5.26 (tt, J=6.1, 3.3 Hz, 1H), 4.38-4.25 (m, 3H), 4.25-4.10 (m, 5H), 4.05-3.88 (m, 2H), 3.54-3.34 (m, 19H), 3.29 (t, J=6.5 Hz, 2H), 3.25 (t, J=5.5 Hz, 2H), 3.02 (td, J=8.9, 4.1 Hz, 1H), 2.73 (dt, J=12.8, 4.7 Hz, 1H), 2.54 (dd, J=12.7, 1.9 Hz, 1H), 2.09 (t, J=7.4 Hz, 2H), 1.64-1.41 (m, 6H), 1.40-1.35 (m, 2H), 1.33-1.15 (m, 6H), 0.55 (s, 3H), 0.46 (s, 3H). ¹³C NMR (CD₃OD, 101 MHz, 320 K) δ 176.07 (C), 171.56 (C), 170.37 (d, ²J_{CF}=24.0 Hz, C), 168.66 (C), 165.98 (C), 152.01 (C), 151.24 (C), 141.72 (C), 138.96 (C), 134.26 (C), 133.81 (C), 129.76 (C), 129.36 (CH), 127.01 (CH), 124.76 (CH), 117.54 (CH), 114.38 (CH), 92.34 (d, ¹J_{CF}=221.4 Hz, C), 84.29 (d, ¹J_{CF}=202.7 Hz, CH), 72.15 (CH₂), 71.34 (CH₂), 71.32 (CH₂), 71.26 (CH₂), 71.14 (CH₂), 70.68 (CH₂), 70.36 (CH₂), 70.29 (CH₂), 63.36 (CH), 62.29 (d, ²J_{CF}=25.0 Hz, CH₂), 61.63 (CH), 60.59 (d, ²J_{CF}=24.1 Hz, CH₂), 56.90 (CH), 45.67 (CH₂), 41.17 (CH₂), 40.98 (CH₂), 40.35 (CH₂), 40.33 (CH₂), 36.77 (CH₂), 33.69 (CH₂), 30.41 (CH₂), 29.73 (CH₂), 29.48 (CH₂), 27.65 (CH₂), 26.77 (CH₂), 26.39 (CH₂), −0.14 (CH₃), −1.19 (CH₃). Two aromatic quaternary carbons were not observed. ¹⁹F NMR (CD₃OD, 376 MHz) δ −75.55, −161.83, −180.00. Analytical HPLC: t_{R}=11.6 min, 99.1% purity (5-95% MeCN/H₂O linear gradient over 15 min with constant 0.1% v/v TFA, 1 mL/min flow rate, detection at 254 nm). HRMS (ESI) calculated for C₅₆H₇₅N₇O₁₀F₂SClSiNa [M+Na]⁺=1160.4536, found 1160.4545.

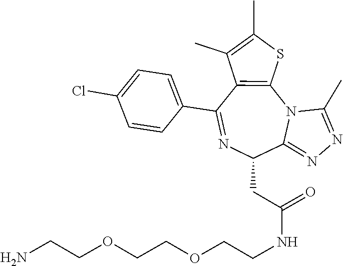 [SMILES: Cc1sc2c(c1C)C(c1ccc(Cl)cc1)=N[C@@H](CC(=O)NCCOCCOCCN)c1nnc(C)n1-2]

(+)-JQ1-PEG2-amine (16). (+)-JQ1-CO₂H (S16, 200 mg, 0.5 mmol, 1 equiv) was dissolved in DMF (4 mL). To this solution were added DIEA (448 μL, 1.25 mmol, 5 equiv), HATU (284 mg, 0.75 mmol, 1.5 equiv), and tert-butyl (2-(2-(2-aminoethoxy)ethoxy)ethyl)carbamate (S17, 186 mg, 0.75 mmol, 1.5 equiv). The reaction mixture was stirred at ambient temperature for 16 h. The solvent was removed under reduced pressure and resuspended in DCM (8 mL). TFA (2.5 mL) was added, and the reaction mixture was stirred at ambient temperature for 8 h. Volatiles were removed under reduced pressure and the product was purified by preparative HPLC using a 30-95% CH₃CN/H₂O linear gradient with a constant 0.1% v/v TFA. Product-containing fractions were combined and lyophilized to obtain 16 as a yellow sticky solid (5×TFA salt, 198.8 mg, 47.9% over two steps). The molar equivalents of TFA were determined via ¹⁹F NMR by using fluorobenzene as an internal standard. ¹H NMR (CD₃OD, 400 MHz) δ 7.49-7.46 (m, 2H), 7.45-7.40 (m, 2H), 4.72 (dd, J=8.3, 6.0 Hz, 1H), 3.74-3.70 (m, 2H), 3.68 (s, 4H), 3.62 (t, J=5.6 Hz, 2H), 3.54-3.34 (m, 4H), 3.13 (t, J=5.0 Hz, 2H), 2.76 (s, 3H), 2.46 (s, 3H), 1.70 (s, 3H). ¹³C NMR (CD₃OD, 101 MHz) δ 172.77 (C), 166.59 (C), 156.89 (C), 152.43 (C), 138.26 (C), 137.80 (C), 133.69 (C), 133.47 (C), 132.16 (C), 131.99 (C), 131.44 (CH), 129.86 (CH), 71.38 (CH₂), 71.36 (CH₂), 70.65 (CH₂), 67.91 (CH₂), 54.99 (CH), 40.69 (CH₂), 40.40 (CH₂), 38.51 (CH₂), 14.39 (CH₃), 12.94 (CH₃), 11.56 (CH₃); TFA peaks: 161.02 (q, ²J_{CF}=38.1 Hz, C), 117.34 (q, ¹J_{CF}=288.4 Hz, C). HRMS (ESI) calculated for C₂₅H₃₂ClN₆O₃S [M+H]⁺=531.1940, found 531.1939.

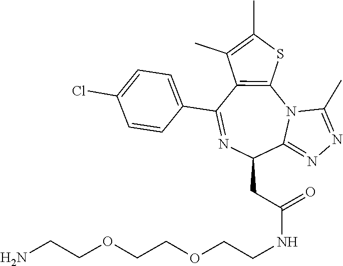 [SMILES: Cc1sc2c(c1C)C(c1ccc(Cl)cc1)=N[C@H](CC(=O)NCCOCCOCCN)c1nnc(C)n1-2]

(−)-JQ1-PEG₂-amine (S21). To a solution of (−)-JQ1-CO₂^{t}Bu (S19, 248.8 mg, 0.54 mmol, 1 equiv) in DCM (10 mL) was added TFA (1 mL). This light orange solution was stirred at ambient temperature for 24 h. The solvent was removed under reduced pressure and the crude was dissolved in DMF (3 mL). To this solution were successively added DIEA (976 μL, 5.44 mmol, 10 equiv), HATU (311 mg, 0.82 mmol, 1.5 equiv), and S17 (203 mg, 0.82 mmol, 1.5 equiv). The reaction mixture was stirred at ambient temperature for 18 h. The solvent was removed under reduced pressure and the oil was resuspended in DCM (4 mL), and TFA (1 mL) was added. The reaction mixture was stirred at ambient temperature for 18 h. The solvent was removed under reduced pressure and the product was purified by preparative HPLC using a 5-95% CH₃CN/H₂O linear gradient with a constant 0.1% v/v TFA. Product-containing fractions were combined and lyophilized to obtain S21 as a yellow sticky solid (5×TFA salt, 350.7 mg, 58.5% over three steps). The molar equivalents of TFA were determined via ¹⁹F NMR by using fluorobenzene as an internal standard. ¹H NMR (CD₃OD, 400 MHz) δ 7.49-7.44 (m, 2H), 7.43-7.42 (m, 2H), 4.71 (dd, J=8.3, 6.0 Hz, 1H), 3.74-3.70 (m, 2H), 3.68 (s, 4H), 3.62 (t, J=5.6 Hz, 2H), 3.53-3.34 (m, 4H), 3.13 (t, J=5.0 Hz, 2H), 2.75 (s, 3H), 2.46 (s, 3H), 1.70 (s, 3H). ¹³C NMR (CD₃OD, 101 MHz) δ 172.68 (C), 166.65 (C), 156.81 (C), 152.51 (C), 138.32 (C), 137.64 (C), 133.87 (C), 133.41 (C), 132.22 (C), 131.97 (C), 131.49 (CH), 129.86 (CH), 71.33 (CH₂), 70.62 (CH₂), 67.89 (CH₂), 54.90 (CH), 40.65 (CH₂), 40.39 (CH₂), 38.38 (CH₂), 14.40 (CH₃), 12.96 (CH₃), 11.55 (CH₃); TFA peaks: 161.41 (q, ²J_{CF}=38.3 Hz, C), 158.94 (q, ²J_{CF}=41.3 Hz, C), 117.34 (q, ¹J_{CF}=289.0 Hz, C), 116.37 (q, ¹J_{CF}=284.4 Hz, C). HRMS (ESI) calculated for C₂₅H₃₂ClN₆O₃S [M+H]⁺=531.1940, found 531.1945.

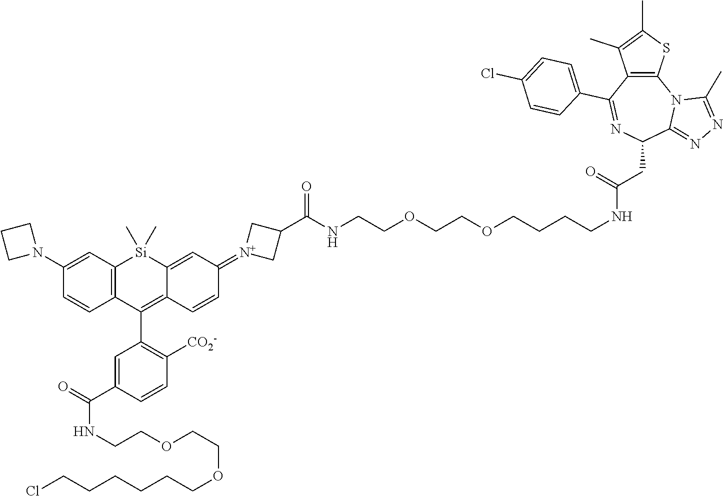

(+)-JQ1-JF₆₄₆-HaloTag® ligand (17). The TFA salt of 9 (23 mg, 27 μmol, 1 equiv) was dissolved in DMF (3 mL). To this solution were added DIEA (49 μL, 267 μmol, 10 equiv) and HATU (12.2 mg, 32 μmol, 1.2 equiv). The reaction mixture was stirred at ambient temperature for 5 min, after which 16 (44.1 mg, 40 μmol, 1.5 equiv) was added. The reaction mixture was stirred for a further 16 h at ambient temperature. The solvent was removed under reduced pressure, and the product was purified by preparative HPLC using a 40-60% CH₃CN/H₂O linear gradient with a constant 0.1% v/v TFA. Product-containing fractions were combined and lyophilized to obtain 17 as a blue solid (4×TFA salt, 29 mg, 63.2%). The molar equivalents of TFA were determined via ¹⁹F NMR by using fluorobenzene as an internal standard. ¹H NMR (CD₃OD, 400 MHz; rotamers observed) δ 8.27 (dd, J=8.2, 2.9 Hz, 1H), 8.11 (dt, J=8.2, 2.0 Hz, 1H), 7.69 (dt, J=7.3, 3.7 Hz, 1H), 7.41 (dd, J=8.6, 2.9 Hz, 2H), 7.36 (d, J=8.3 Hz, 2H), 6.93-6.76 (m, 4H), 6.34 (dd, J=9.3, 2.6 Hz, 1H), 6.15-6.04 (m, 1H), 4.64 (dd, J=8.8, 5.5 Hz, 1H), 4.36-4.18 (m, 8H), 3.66-3.55 (m, 16H), 3.53-3.48 (m, 3H), 3.46-3.40 (m, 7H), 3.36-3.33 (m, 1H), 2.61 (d, J=8.9 Hz, 3H), 2.52 (p, J=7.7 Hz, 2H), 2.43 (d, J=2.9 Hz, 3H), 1.74-1.66 (m, 5H), 1.50 (p, J=6.8 Hz, 2H), 1.38 (q, J=7.6 Hz, 2H), 1.33 (dd, J=9.0, 5.8 Hz, 2H), 0.56 (d, J=8.3 Hz, 3H), 0.52 (d, J=3.5 Hz, 3H). ¹³C NMR (CD₃OD, 101 MHz; rotamers observed) δ 173.91 (C), 172.76 (C), 168.09 (C), 167.99 (C), 166.21 (C), 156.98 (C), 154.20 (C), 153.51 (C), 152.24 (C), 139.15 (C), 138.06 (C), 137.91 (C), 133.45 (C), 133.36 (C), 132.06 (C), 132.03 (C), 131.94 (C), 131.90 (C), 131.35 (CH), 129.81 (CH), 129.53 (CH), 128.97 (CH), 119.77 (CH), 119.15 (CH), 113.28 (CH), 112.74 (CH), 72.12 (CH₂), 71.40 (CH₂), 71.34 (CH₂), 71.21 (CH₂), 71.14 (CH₂), 70.61 (CH₂), 70.43 (CH₂), 70.38 (CH₂), 55.28 (CH₂), 55.09 (CH), 53.28 (CH₂), 45.74 (CH₂), 41.16 (CH₂), 40.66 (CH₂), 40.56 (CH₂), 38.71 (CH₂), 34.78 (CH), 33.71 (CH₂), 30.45 (CH₂), 27.68 (CH₂), 26.43 (CH₂), 16.97 (CH₂), 14.44 (CH₃), 12.98 (CH₃), 11.64 (CH₃), −0.81 (CH₃), −1.75 (CH₃). Five aromatic quaternary carbons were not observed. Analytical HPLC: t_{R}=13.8 min, 96.5% purity (10-95% CH₃CN/H₂O linear gradient over 20 min with constant 0.1% v/v TFA, 1 mL/min flow rate, detection at 650 nm). HRMS (ESI) calculated for C₆₅H₇₈N₉O₉SCl₂Si [M+H]⁺=1258.4790, found 1258.4790.

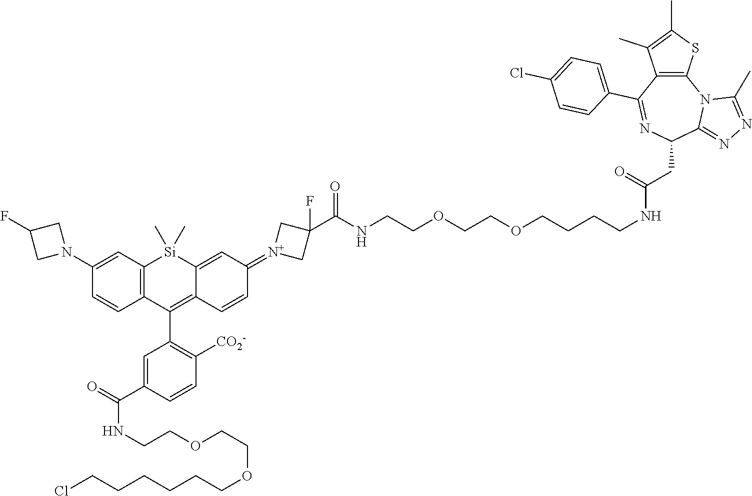

(+)-JQ1-JF₆₃₅-HaloTag® ligand (18). The TFA salt of 10 (61.6 mg, 69 μmol, 1 equiv) was dissolved in DMF (3 mL). To this solution were added DIEA (121 μL, 109 μmol, 10 equiv), EDC.HCl (25.8 mg, 135 μmol, 2 equiv), HATU (51.3 mg, 135 μmol, 2 equiv). The reaction mixture was stirred at ambient temperature for 10 min, after which 16 (55.1 mg, 50 μmol, 0.72 equiv) was added. The reaction mixture was stirred for a further 16 h at ambient temperature. The solvent was removed under reduced pressure, and the product was purified by preparative HPLC using a 40-65% CH₃CN/H₂O linear gradient with a constant 0.1% v/v TFA. Product-containing fractions were combined and lyophilized to obtain 18 as a blue solid (4×TFA salt, 28.8 mg, 32.9%). The molar equivalents of TFA were determined via ¹⁹F NMR by using fluorobenzene as an internal standard. ¹H NMR (CD₃OD, 400 MHz, 320 K) δ 8.07 (qd, J=8.3, 3.6 Hz, 2H), 7.67 (d, J=6.4 Hz, 1H), 7.43 (d, J=8.3 Hz, 2H), 7.39-7.33 (m, 2H), 6.90-6.75 (m, 4H), 6.38 (dd, J=8.9, 2.6 Hz, 1H), 6.30 (td, J=8.7, 2.6 Hz, 1H), 5.53-5.46 (m, 0.5H), 5.39-5.32 (m, 0.5H), 4.70-4.58 (m, 1H; buried under the water peak), 4.48-4.27 (m, 4H), 4.26-4.14 (m, 2H), 4.14-4.00 (m, 2H), 3.65-3.56 (m, 12H), 3.55-3.50 (m, 4H), 3.50-3.33 (m, 10H), 2.66 (d, J=8.3 Hz, 3H), 2.40 (d, J=3.5 Hz, 3H), 1.73-1.63 (m, 5H), 1.47 (p, J=6.8 Hz, 2H), 1.37 (p, J=7.0 Hz, 2H), 1.29 (q, J=8.3 Hz, 2H), 0.61 (d, J=5.0 Hz, 3H), 0.53 (d, J=4.9 Hz, 3H). ¹³C NMR (CD₃OD, 101 MHz; rotamers observed) δ 172.69 (C), 170.72 (C), 170.20 (d, ²J_{CF}=21.3 Hz, C), 168.40 (C), 166.32 (C), 156.89 (C), 152.46 (C), 152.28 (C), 151.60 (C), 141.04 (C), 138.11 (C), 137.73 (C), 133.54 (C), 133.39 (C), 132.99 (C), 132.12 (C), 131.90 (C), 131.44 (CH), 129.80 (CH), 129.31 (CH), 128.04 (CH), 125.80 (CH), 118.87 (CH), 118.15 (CH), 114.27 (CH), 114.07 (CH), 92.03 (d, ¹J_{CF}=221.9 Hz, C), 84.12 (d, ¹J_{CF}=202.6 Hz, C), 72.11 (CH₂), 71.36 (CH₂), 71.26 (CH₂), 71.19 (CH₂), 71.11 (CH₂), 70.66 (CH₂), 70.33 (CH₂), 70.20 (CH₂), 62.30 (d, ²J_{CF}=25.1 Hz, CH₂), 60.62 (d, ²J_{CF}=24.5 Hz, CH₂), 54.99 (CH), 45.74 (CH₂), 41.14 (CH₂), 40.52 (CH₂), 40.28 (CH₂), 38.52 (CH₂), 33.68 (CH₂), 30.41 (CH₂), 27.66 (CH₂), 26.39 (CH₂), 14.46 (CH₃), 12.99 (CH₃), 11.64 (CH₃), −0.10 (CH₃), −1.30 (CH₃). TFA peaks: 161.33 (q, ²J_{CF}=37.5 Hz, C), 158.94 (q, ²J_{CF}=41.4 Hz, C), 116.04 (q, ¹J_{CF}=285.1 Hz, C). Five aromatic quaternary carbons were not observed. ¹⁹F NMR (CD₃OD, 376 MHz) δ −75.14, −75.62, −161.97, −179.86. Analytical HPLC: t_{R}=14.7 min, 98.4% purity (30-95% CH₃CN/H₂O linear gradient over 20 min with constant 0.1% v/v TFA, 1 mL/min flow rate, detection at 650 nm). HRMS (ESI) calculated for C₆₅H₇₆N₉O₉F₂SCl₂Si [M+H]⁺=1294.4596, found 1294.4609.

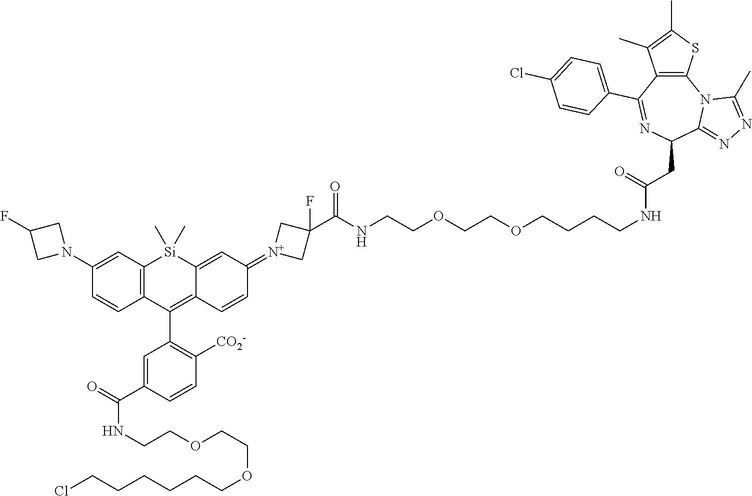

(−)-JQ1-JF₆₃₅-HaloTag® ligand (19). The TFA salt of 10 (8.5 mg, 11 μmol, 1 equiv) was dissolved in DMF (2 mL). To this solution were added DIEA (20 μL, 109 μmol, 10 equiv), S22 (30 mg, 26 μmol, 1.7 equiv), EDC.HCl (6.1 mg, 32 μmol, 3 equiv), and HATU (12.2 mg, 32 μmol, 3 equiv). The reaction mixture was stirred at ambient temperature for 16 h. The solvent was removed under reduced pressure, and the product was purified by preparative HPLC using a 5-95% CH₃CN/H₂O linear gradient with a constant 0.1% v/v TFA. Product-containing fractions were combined and lyophilized to obtain 19 as a blue solid (TFA salt, 8.8 mg, 52.5%). ¹H NMR (CD₃OD, 400 MHz) δ 8.36 (br s, 1H), 8.07-8.03 (m, 2H), 7.69-7.66 (m, 1H), 7.42-7.35 (m, 4H), 6.84-6.79 (m, 3H), 6.74 (d, J=8.8 Hz, 1H), 6.40 (ddd, J=8.9, 2.7, 1.6 Hz, 1H), 6.29-6.23 (m, 1H), 5.52-5.48 (m, 0.5H), 5.39-5.34 (m, 0.5H), 4.66-4.61 (m, 1H), 4.45-3.93 (m, 8H), 3.67-3.41 (m, 22H), 3.39 (t, J=6.5 Hz, 2H), 3.29-3.27 (m, 2H), 2.65 (2×s, 3H), 2.41 (2×s, 3H), 1.73-1.60 (m, 5H), 1.46 (p, J=6.8 Hz, 2H), 1.40-1.33 (m, 2H), 1.31-1.27 (m, 2H), 0.62 (2×s, 3H), 0.53 (2×s, 3H). Analytical HPLC: t_{R}=15.7 min, 97.7% purity (5-95% CH₃CN/H₂O linear gradient over 20 min with constant 0.1% v/v TFA, 1 mL/min flow rate, detection at 254 nm). HRMS (ESI) calculated for C₆₅H₇₆N₉O₉F₂SCl₂Si [M+H]⁺=1294.4596, found 1294.4597.

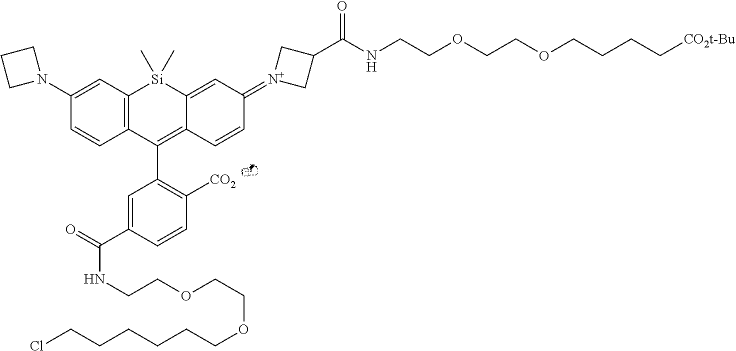

t-BuO₂C-PEG₂-JF₆₄₆-HaloTag® ligand (21). The TFA salt of 3″-carboxy-JF₆₄₆-HaloTag® ligand¹⁴ (9; 45.8 mg, 61.4 μmol) and EDC.HCl (17.6 mg, 92.0 μmol, 1.5 eq) were combined in CH₂Cl₂ (3 mL); tert-butyl 3-(2-(2-aminoethoxy)ethoxy)propanoate (20; 17.2 mg, 73.6 μmol, 1.2 eq) and DMAP (1.5 mg, 12.3 μmol, 0.2 eq) were added, and the reaction was stirred at room temperature for 1 h. It was subsequently diluted with 10% w/v citric acid and extracted with CH₂Cl₂ (3×). The combined organic extracts were dried over anhydrous MgSO₄, filtered, and concentrated in vacuo. Silica gel chromatography (0-10% MeOH/CH₂Cl₂, linear gradient) afforded 53.4 mg (91%) of the title compound as a green solid. ¹H NMR (CDCl₃, 400 MHz) δ 8.01-7.96 (m, 1H), 7.91 (dd, J=8.0, 1.4 Hz, 1H), 7.67 (d, J=1.2 Hz, 1H), 6.78-6.74 (m, 2H), 6.69 (d, J=2.6 Hz, 1H), 6.65 (d, J=2.6 Hz, 1H), 6.45 (t, J=5.6 Hz, 1H), 6.31-6.28 (m, 1H), 6.28-6.25 (m, 1H), 4.10-3.99 (m, 4H), 3.89 (t, J=7.3 Hz, 4H), 3.73 (t, J=6.2 Hz, 2H), 3.65-3.59 (m, 11H), 3.58-3.53 (m, 4H), 3.53-3.46 (m, 4H), 3.39 (t, J=6.7 Hz, 2H), 2.49 (t, J=6.2 Hz, 2H), 2.37 (p, J=7.2 Hz, 2H), 1.73 (p, J=6.8 Hz, 2H), 1.51 (p, J=6.9 Hz, 2H), 1.45 (s, 9H), 1.39 (p, J=7.6 Hz, 2H), 1.34-1.23 (m, 2H), 0.63 (s, 3H), 0.57 (s, 3H); MS (ESI) calcd for C₅₁H₇₀ClN₄O₁₀Si [M+H]⁺ 961.5, found 961.4.

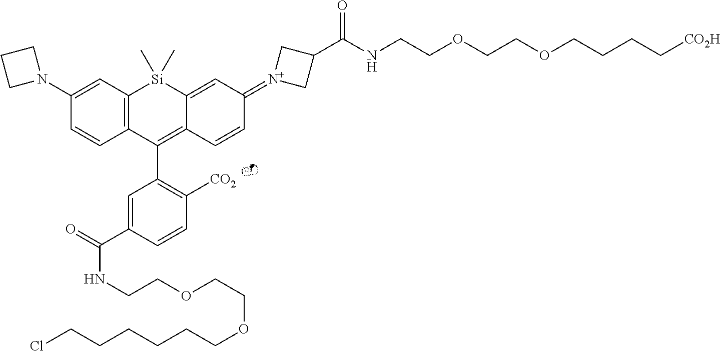

HO₂C-PEG₂-JF₆₄₆-HaloTag® ligand (22). t-BuO₂C-PEG₂-JF₆₄₆-HaloTag® ligand (21; 71 mg, 73.8 μmol) was taken up in CH₂Cl₂ (3.75 mL), and trifluoroacetic acid (0.75 mL) was added. The reaction was stirred at room temperature for 6.5 h. Toluene (4.5 mL) was added; the reaction mixture was concentrated to dryness and then azeotroped with MeOH three times to provide the title compound as a dark blue solid (75 mg, quantitative, TFA salt). The material was used without further purification. ¹H NMR (CD₃OD, 400 MHz) δ 8.28 (d, J=8.2 Hz, 1H), 8.11 (dd, J=8.2, 1.8 Hz, 1H), 7.69 (d, J=1.8 Hz, 1H), 6.95 (d, J=2.6 Hz, 2H), 6.91 (t, J=9.3 Hz, 2H), 6.36 (dt, J=9.3, 2.2 Hz, 2H), 4.46-4.26 (m, 8H), 3.74 (t, J=6.2 Hz, 2H), 3.66-3.56 (m, 13H), 3.52 (t, J=6.6 Hz, 2H), 3.46-3.39 (m, 4H), 2.58-2.51 (m, 4H), 1.78-1.66 (m, 2H), 1.56-1.47 (m, 2H), 1.44-1.29 (m, 6H), 0.59 (s, 3H), 0.55 (s, 3H); MS (ESI) calcd for C₄₇H₆₂ClN₄O₁₀Si [M+H]⁺ 905.4, found 905.3.

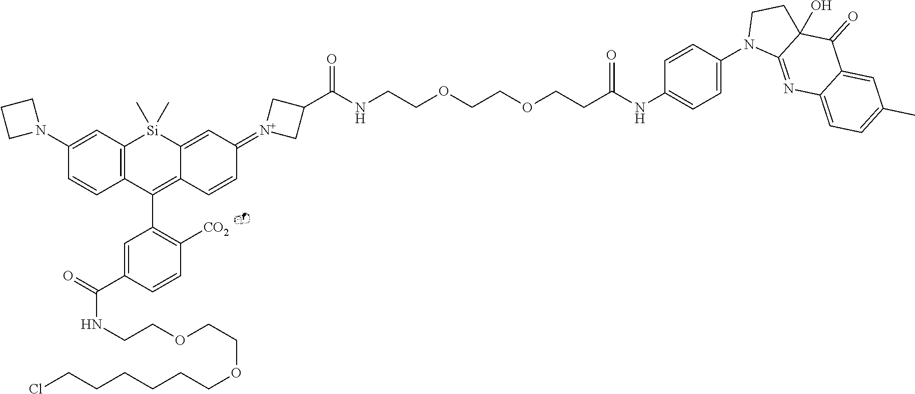

Blebbistain-JF₆₄₆-HaloTag® ligand (24). HO₂C-PEG₂-JF₆₄₆-HaloTag® ligand (22; 19.9 mg, 19.5 μmol, 1.2 eq) and HATU (7.4 mg, 19.5 μmol, 1.2 eq) were combined in DMF (2 mL); (S)-1-(4-aminophenyl)-3a-hydroxy-6-methyl-1,2,3,3a-tetrahydro-4H-pyrrolo[2,3-b]quinolin-4-one (“(S)-4-amino-blebbistatin”; 23; 5 mg, 16.3 μmol, 1 eq) and DIEA (8.5 μL, 48.8 μmol, 3 eq) were added, and the reaction was stirred at room temperature for 4.5 h. It was subsequently concentrated in vacuo and purified by reverse phase HPLC (30-45% MeCN/H₂O, linear gradient, with constant 0.1% TFA additive). The pooled product fractions were partially concentrated to remove MeCN, neutralized with saturated NaHCO₃, and extracted with CH₂Cl₂ (3×). The combined organic layers were dried over anhydrous MgSO₄, filtered, and evaporated to yield 10 mg (52%) of the title compound as a pale green solid. ¹H NMR (CDCl₃, 400 MHz) δ 8.37 (s, 0.5H), 8.30 (s, 0.5H), 7.98 (s, 2H), 7.89-7.83 (m, 2H), 7.74 (s, 0.5H), 7.71 (s, 0.5H), 7.55-7.49 (m, 3H), 7.36-7.29 (m, 1H), 7.25-7.22 (m, 1H), 7.09 (d, J=8.1 Hz, 1H), 6.73-6.61 (m, 3H), 6.59 (d, J=2.7 Hz, 0.5 H), 6.53 (d, J=2.7 Hz, 0.5H), 6.35 (q, J=6.9 Hz, 1H), 6.23 (dt, J=8.7, 2.6 Hz, 1H), 6.14 (dd, J=8.7, 2.6 Hz, 0.5H), 6.04 (dd, J=8.6, 2.6 Hz, 0.5H), 4.51 (s, 0.5H), 4.37 (s, 0.5H), 4.02-3.92 (m, 1H), 3.89-3.79 (m, 9H), 3.71-3.54 (m, 13H), 3.50-3.44 (m, 5H), 3.34 (q, J=6.9 Hz, 2H), 3.23 (q, J=7.2 Hz, 1H), 2.63-2.51 (m, 2H), 2.42-2.32 (m, 2H), 2.28 (d, J=8.6 Hz, 4H), 2.04-1.88 (m, 2H), 1.70 (p, J=6.5 Hz, 2H), 1.47 (p, J=6.2 Hz, 2H), 1.42-1.33 (m, 1H), 1.31-1.26 (m, 4H), 0.60-0.49 (m, 6H); Analytical HPLC: t_{R}=11.6 min, 94% purity (10-95% MeCN/H₂O, linear gradient, with constant 0.1% v/v TFA additive; 20 min run; 1 mL/min flow; ESI; positive ion mode; detection at 650 nm); MS (ESI) calcd for C₆₅H₇₇ClN₇O₁₁Si [M+H]⁺ 1194.5, found 1194.5.

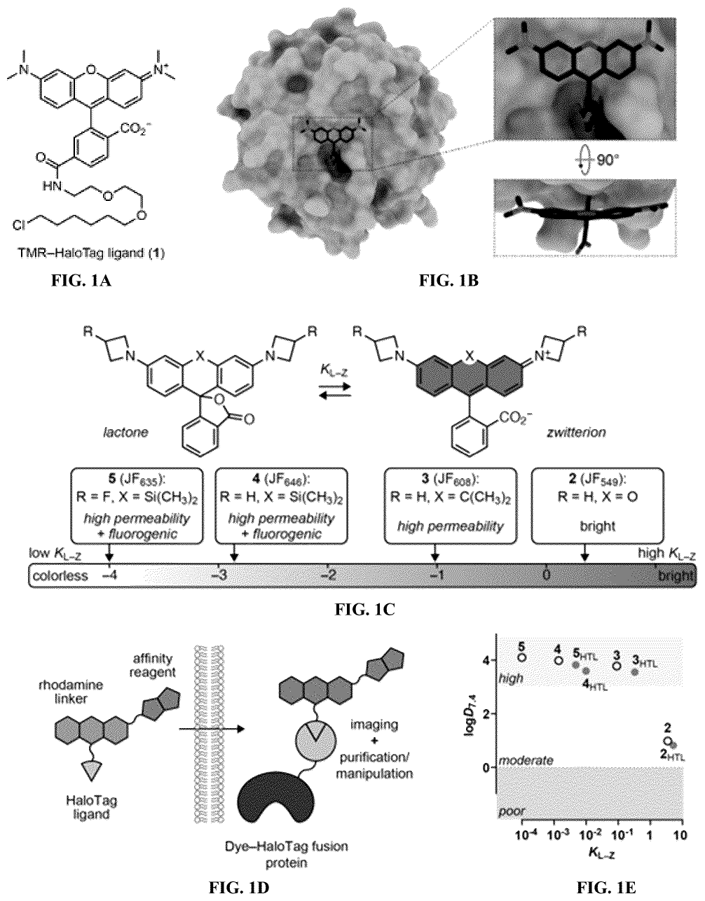

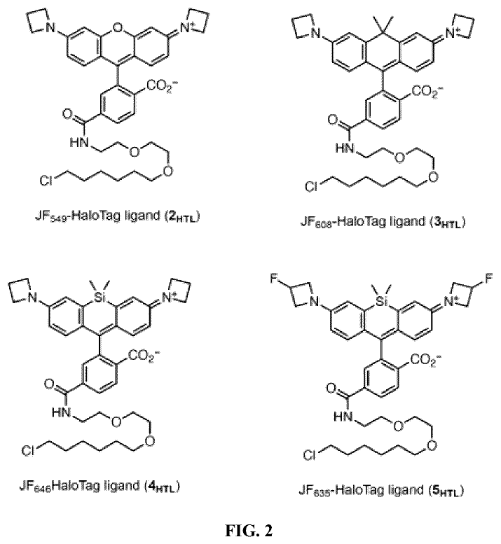

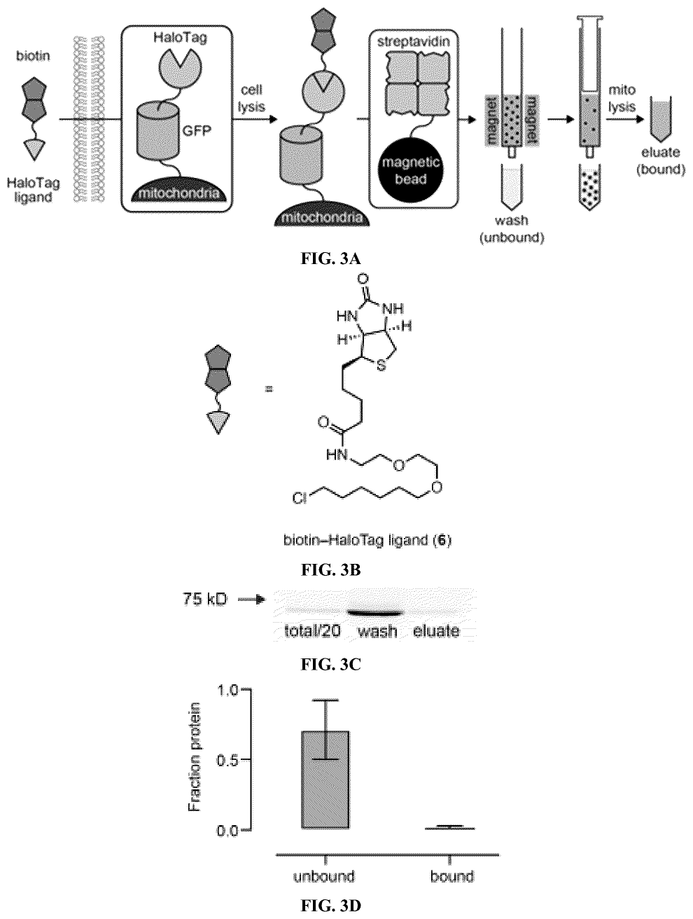

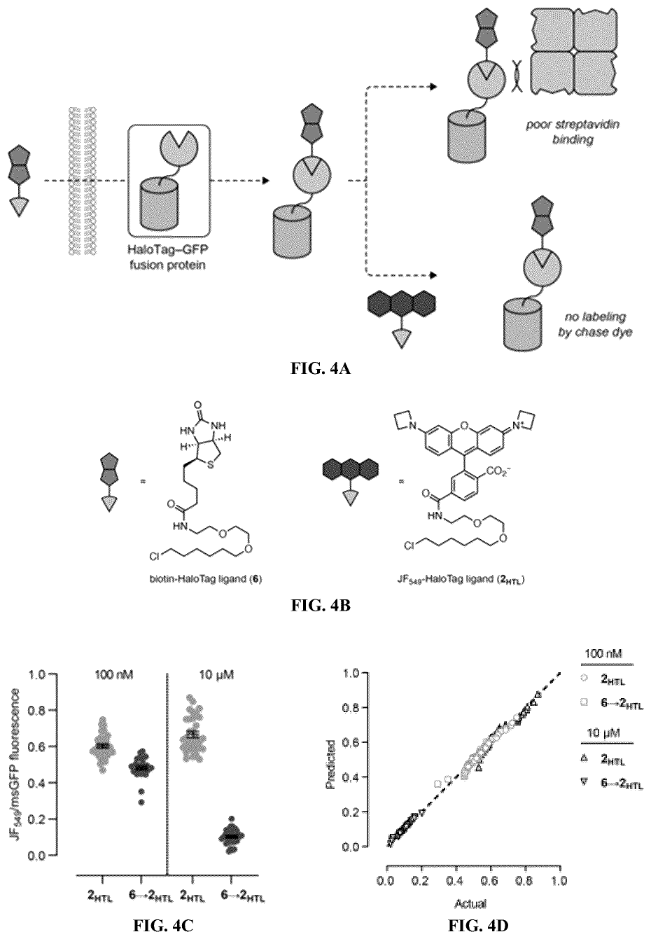

Evaluation of biotin-HaloTag® ligand. A live cell permeable biotin reagent was saught for affinity pulldown of HaloTag® fusion proteins, given the broad utility of biotin conjugates in biochemistry and cell biology. To evaluate the performance of biotin-containing HaloTag® ligand, an assay was developed (FIG. 3A) where the HaloTag® is expressed as a fusion with monomeric superfolder green fluorescent protein (msGFP); the construct also included an OMP25 sequence to localize the fusion protein to the outer mitochondrial membrane, allowing straightforward imaging and affinity capture experiments.²⁸⁻²⁹ HEK293T cells expressing this fusion protein were incubated with biotin-containing HaloTag® ligands followed by washing and cell lysis. The crude supernatant was incubated with streptavidin-coated magnetic microbeads, applied to a magnetic column, and washed. The magnet was removed, and the bead-bound mitochondria were eluted, lysed, and the resulting solution was analyzed by sodium dodecyl sulfate-polyacrylamide gel electrophoresis (SDS-PAGE) and in-gel fluorescence. To minimize dilution of the sample only the initial bead-bound fraction was collected, leaving some material on the column. The commercial biotin-HaloTag® ligand was tested (6; FIG. 3B), which has the standard HaloTag® ligand found in compound 1 or 2_{HTL}-5_{HTL}. (FIG. 1A) directly attached to the biotin carboxyl group. Pulse-chase experiments revealed compound 6 labels the HaloTag®-msGFP fusion in living cells when applied at μM concentrations (FIG. 4A-4D). Compound 6 has substantially lower labeling kinetics compared to dye-based ligands such as 1,³⁰ and its polar nature could decrease cell-permeability as suggested by poor labeling of HaloTag®-msGFP fusion at nanomolar concentration (FIG. 4A-4D). In addition to lower labeling efficiency, the standard PEG2 linker in compound 6 is too short to allow binding of the biotin-HaloTag® conjugate to streptavidin, which prevents purification of HaloTag®-expressing mitochondria from cells using streptavidin beads (FIG. 3C-3D). This is probably due to the close association of biotin and HaloTag® (FIG. 1B). Unfortunately, attempts to remedy this problem by incorporating a longer PEG linker yields a biotin HaloTag® ligand with very poor cell-permeability,³¹ thereby rendering it unsuitable for experiments in live cells.

Design and synthesis of biotin-rhodamine-HaloTag® ligands. The problems with the existing biotin-containing HaloTag® ligands could be remedied by incorporating a rhodamine linker into the chemical structure instead of a longer PEG linker (FIG. 1D). It was contemplated to use JF dyes 2-5 as the linkers, given they span the K_{L-Z} scale. JF₅₄₉ (2) is structurally similar to tetramethylrhodamine (TMR), but contains four-membered azetidine rings in place of the N,N-dimethylamino groups; this net increase of two carbon atoms greatly increases brightness and photostability.³² JF₆₀₈ (3) is a carborhodamine where the xanthene oxygen in 2 is replaced with a dimethylcarbon group.¹⁷ This substitution elicits a ˜60-nm bathochromic shift in absorption maximum (λ_{abs}) and fluorescence emission maximum (λₑₘ). JF₆₄₆ (4) is a Si-rhodamine where the xanthene oxygen in 3 is replaced with a dimethylsilicon group.³³⁻³⁵ This substitution elicits a substantial ˜100-nm bathochromic shift in λ_{abs} and λₑₘ. JF₆₃₅ (5) contains 3-fluoroazetidine substituents that fine-tune the properties of the Si-rhodamine molecule, eliciting a modest 11-nm hypsochromic shift in both λ_{abs} and λₑₘ.²³

In addition to different spectral characteristics, 2-5 also exhibit different K_{L-Z} value. The oxygen-containing JF₅₄₉ (2) has a relatively high K_{L-Z}=3.5, indicating it preferentially adopts the zwitterionic form. The dimethylcarbon substitution in JF₆₀₈ (3) moderately lowers the equilibrium (K_{L-Z}=0.091) and makes it highly cell permeable. The dimethylsilicon substitution in JF₆₄₆ (4) causes a large shift in equilibrium toward the nonfluorescent lactone form (K_{L-Z}=0.0014). The electron-withdrawing fluorine atoms on the azetidine auxochromes in JF₆₃₅ (5) further lowers this equilibrium (K_{L-Z}=0.0001), yielding a dye with low visible absorption even in polar media such as water. Together, the series of JF₅₄₉, JF₆₀₈, JF₆₄₆, and JF₆₃₅ exhibit different chemical properties, allowing evaluation of these rhodamine dyes as linkers with the goal to improve the cell-permeability of biotin-HaloTag® ligand conjugates.

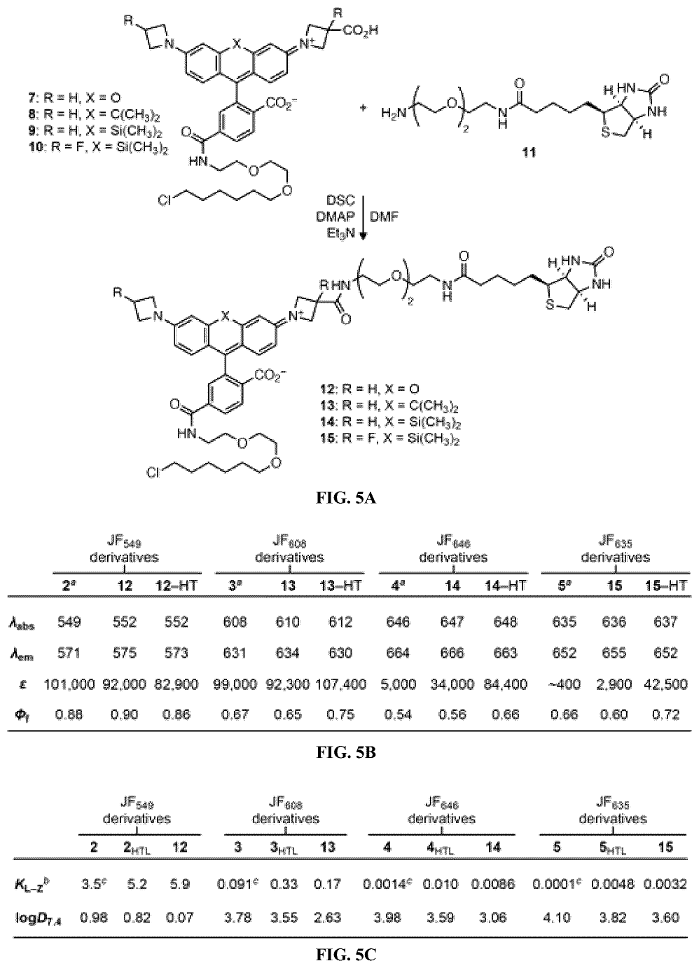

In addition to improving brightness and allowing fine-tuning of spectral properties and K_{L-Z} (e.g., JF₆₃₅, 5), the azetidine motif also allows facile conjugation of chemical groups sing 3-carboxyazetidine derivatives. In previous work, the Ca²⁺ chelator 1,2-bis(o-aminophenoxy)ethane-N,N,N′,N′-tetraacetic acid (BAPTA) was attached to prepare localizable calcium ion indicators useful for subcellular functional imaging.¹⁴ Based on this strategy, biotin conjugates were prepared by using the 3-carboxyazetidine-containing compounds 7-10 with the commercially available biotin-PEG₂-NH₂ (11) to synthesize the biotin-JF-HaloTag® ligand compounds 12-15 (FIG. 5A). The 3″-carboxy-JF₅₄₉-HaloTag® ligand (7) and 3″-carboxy-JF₆₄₆-HaloTag® ligand (9) were prepared as previously described.¹⁴ The JF₆₀₈ and JF₆₃₅ derivative 8 and 10 were synthesized using an analogous sequence in five steps (Scheme 1 and 2). The biotin-JF₅₄₉-HaloTag® ligand (12) is structurally similar to the biotin-TMR-HaloTag® ligand recently reported by Johnsson and coworkers, which showed labeling kinetics that approached those of the unmodified TMR-HaloTag® ligand (1);¹⁷ this compound was not evaluated for intracellular labeling in living cells.

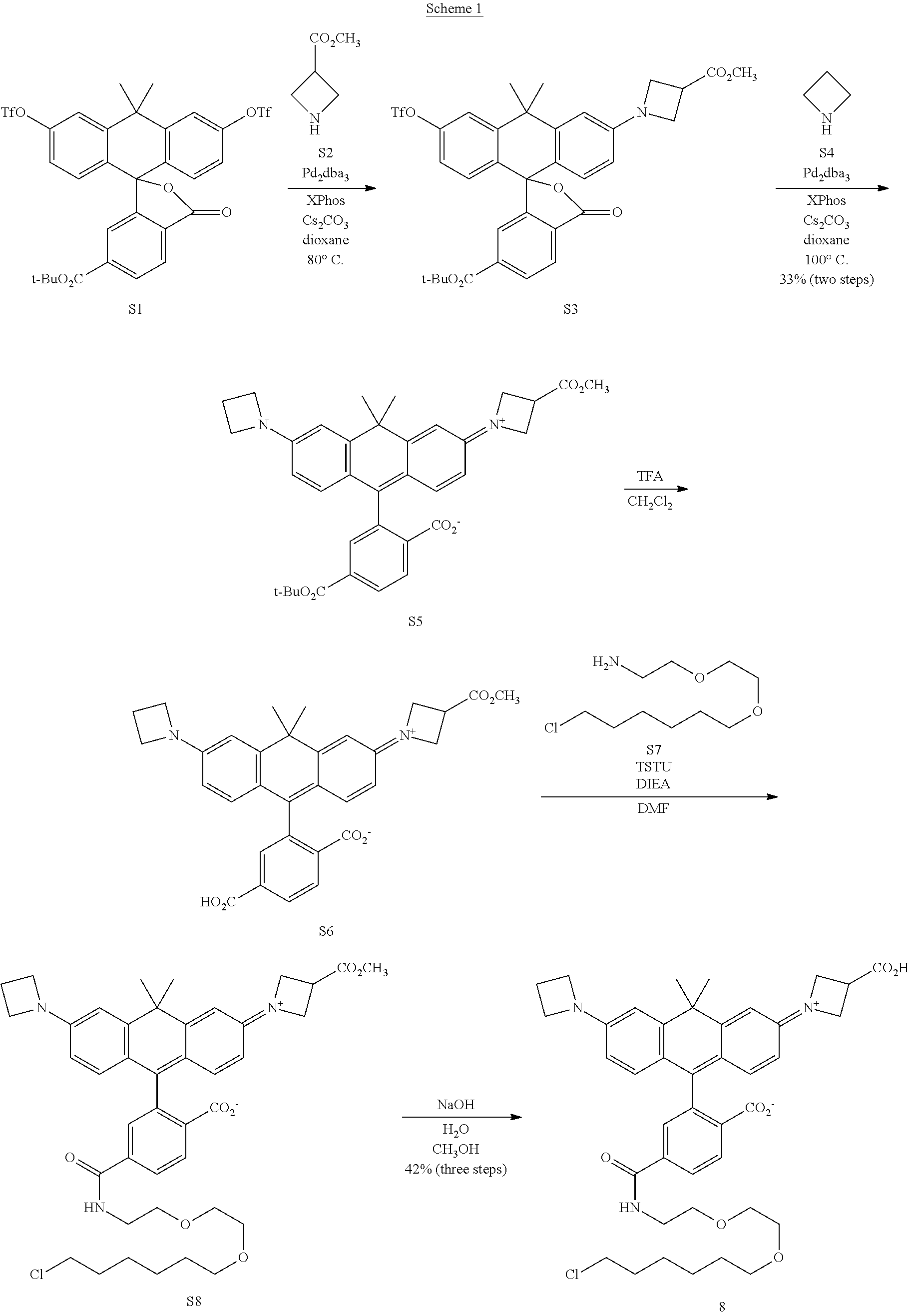

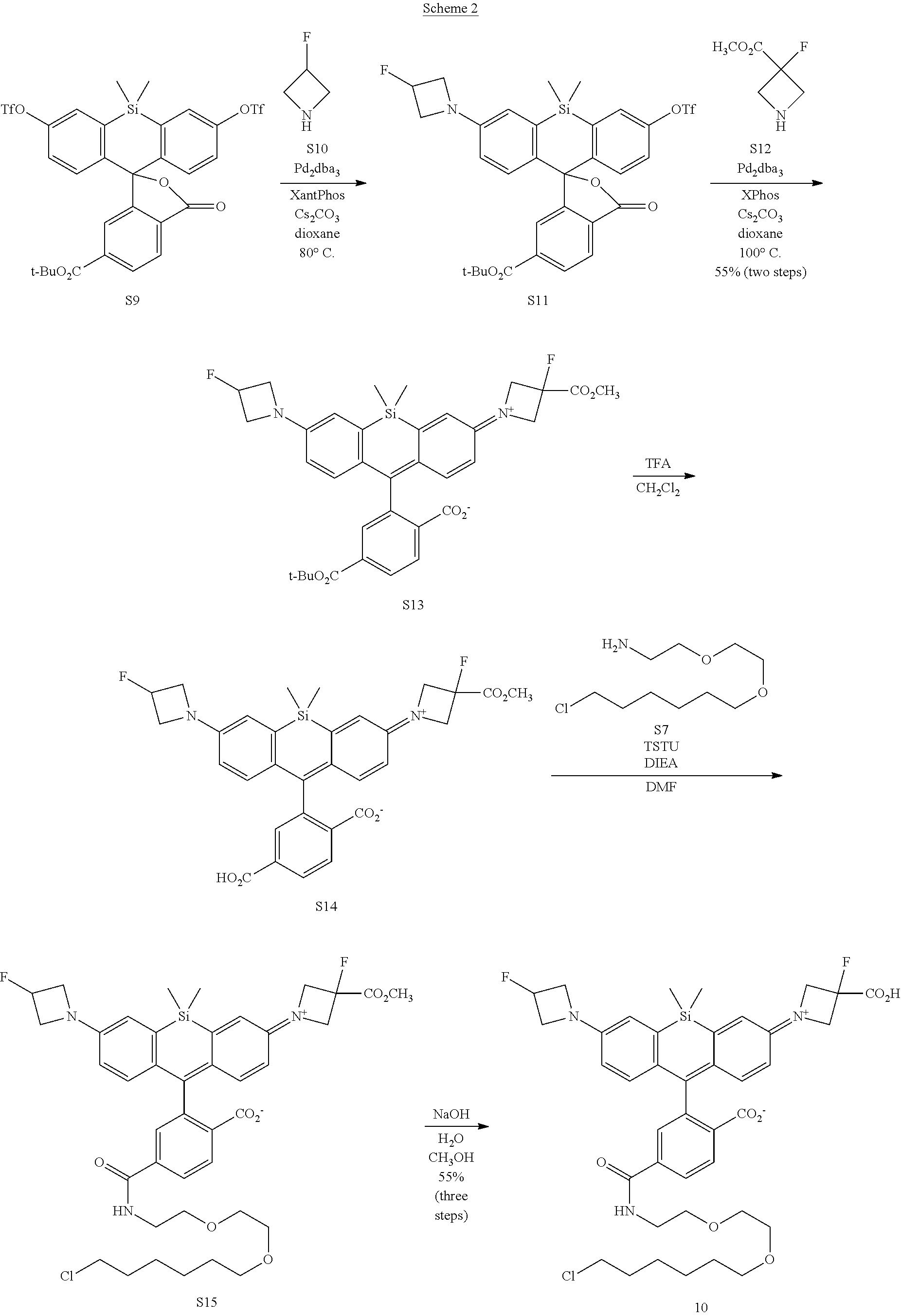

Photophysical properties of biotin-rhodamine-HaloTag® ligands. The spectral properties of the biotin-JF-HaloTag® ligands 12-15 and their HaloTag® conjugates were characterized by comparison to parent dyes 2-5 (FIG. 5B).^{23, 32} The biotin-JF₅₄₉-HaloTag® ligand (12) exhibits similar properties to the parent JF₅₄₉ (2) in aqueous solution, with a slight bathochromic shift (λ_{abs}/λₑₘ=552 nm/575 nm), large extinction coefficient (ε=92,000 M⁻¹cm⁻¹), and high fluorescence quantum yield (Φ_{f}=0.90). Conjugation to the HaloTag® protein has a minimal effect on the spectral properties of 12 (12-HT: λ_{abs}/λₑₘ=552 nm/573 nm, ε=82,900 M⁻¹cm⁻¹, Φ_{f}=0.86). The carborhodamine-based biotin-JF₆₀₈-HaloTag® ligand (13: λ_{abs}/λₑₘ=610 nm/634 nm, ε=92,300 M⁻¹cm⁻¹, Φ_{f}=0.65) also exhibits similar properties to the parent dye 3. Upon binding to the HaloTag® protein (13-HT) the absorption and emission maxima show a minimal shift (λ_{abs}/λₑₘ=612 nm/630 nm) but both the absorptivity and fluorescence quantum yield increase moderately (ε=107,400 M⁻¹cm⁻¹, Φ_{f}=0.75). The Si-rhodamine-based biotin-JF₆₄₆-HaloTag® ligand (14: λ_{abs}/λₑₘ=647 nm/666 nm, Φ_{f}=0.56) also exhibits similar properties to the parent dye 4 but shows a substantially higher absorptivity (ε=34,000 M⁻¹cm⁻¹). Upon binding to the HaloTag® protein (14-HT) the absorption and emission maxima show a minimal shift (λ_{abs}/λₑₘ=648 nm/663 nm) but both the absorptivity and fluorescence quantum yield increase (ε=84,400 M⁻¹cm^{31 1}, Φ_{f}=0.66). The fluorinated biotin-JF₆₃₅-HaloTag® ligand (15) shows a hypsochromic shift in spectral properties (λ_{abs}/λₑₘ=636 nm/655 nm) relative to 14 with a similar quantum yield (Φ_{f}=0.60). Like the parent dye JF₆₃₅, compound 15 exhibits a lower extinction coefficient (ε=2,900 M⁻¹cm⁻¹) compared to 14 due to the fluorine-induced shifts the lactone-zwitterion equilibrium. HaloTag® adduct (15-HT) exhibits similar spectra (λ_{abs}/λₑₘ=637 nm/652 nm) but increased absorptivity (ε=42,500 M⁻¹cm⁻¹) and quantum yield (Φ_{f}=0.72) relative to 5. Overall, the brightness and fluorogenicity pattern of biotin-JF-HaloTag® ligands is similar to the parent JF dyes 2-5.

Chemical properties of biotin-rhodamine-HaloTag® ligands. The K_{L-Z} values of 2-5, 2_{HTL}-5_{HTL}, and 12-15 were measured in 1:1 (v/v) dioxane:water^{13,23} (FIG. 5C). Their logD_{7.4} was also determined using octanol-PBS³⁶. As mentioned above, JF₅₄₉ (2) exhibits a K_{L-Z}=3.5, indicating a strong preference for the polar zwitterionic form. Incorporation of the HaloTag® ligand moiety on JF₅₄₉ (2_{HTL}) further raises the K_{L-Z}=5.2, presumably due to the electron-withdrawing carboxamide para to the carboxylate group on the pendant phenyl ring. The addition of the biotin moiety in 12 further increases the K_{L-Z}=5.9; this larger K_{L-Z} is observed despite the electron-withdrawing nature of the carboxamide group on the azetidine ring, indicating the polar biotin moiety stabilizes the zwitterion form, perhaps through direct interaction with the dye. The trends in K_{L-Z} are mirrored in the logD_{7.4} measurements. The parent dye JF₅₄₉ (2) exhibits a logD_{7.4}=0.98, indicating a preference for lipophilic environments; the JF₅₄₉-HaloTag® ligand (2_{HTL}) is similar with logD_{7.4}=0.82. However, the biotin-JF₅₄₉-HaloTag® ligand (12) is equally distributed between the nonpolar octanol and polar PBS, exhibiting a much lower logD_{7.4}=0.07. Although the relationship between logD_{7.4} and cell-permeability is complicated, cellular entry is optimal when logD_{7.4} is greater than ˜1 but less than 3-5;³⁷⁻³⁹ the permeability of higher molecular weight molecules benefits from higher logD_{7.4} values.⁴⁰ The substantially lower logD_{7.4} of 12, caused by the incorporation of the biotin moiety, falls below this optimal range.

Like the JF₅₄₉ series, the K_{L-Z} values of carborhodamine 3_{HTL} (K_{L-Z}=0.33) and biotin-JF₆₀₈-HaloTag® ligand (13: K_{L-Z}=0.17) were higher than the parent dye 3 (K_{L-Z}=0.091). However, the incorporation of the biotin moiety in 13 decreased K_{L-Z} relative to 3_{HTL}, showing that the electron-withdrawing 3″-carboxamide group overwhelms any effects from the proximity of the biotin moiety in this carborhodamine-based ligand. The parent JF₆₀₈ dye (3) showed logD_{7.4}=3.78. Introduction of the HaloTag® ligand to give 3_{HTL} decreased this value (logD_{7.4}=3.55), and the biotin compound saw a further decrease (logD_{7.4}=2.63). Although a complete inverse correlation between K_{L-Z} and logD_{7.4} is not observed within this compound class, these data show the higher propensity of the JF₆₀₈ unit to adopt the lipophilic lactone form (FIG. 1C) balances the polar biotin-PEG moiety in ligand 13, giving a higher logD_{7.4} value compared to the biotin-JF₅₄₉-HaloTag® ligand (12).

Next, the biotin-JF₆₄₆-HaloTag® ligand (14) and parent JF₆₄₆-HaloTag® ligand (4_{HTL}) were evaluated. Like the JF₅₄₉ and JF₆₀₈ series, the K_{L-Z} values of 4_{HTL} (K_{L-Z}=0.010) and 14 (K_{L-Z}=0.0086) were higher than the parent dye 4 (K_{L-Z}=0.0014). Like JF₆₀₈ series, incorporation of the biotin moiety in 14 decreased K_{L-Z} relative to 4_{HTL}. The parent JF₆₄₆ dye (4) showed logD_{7.4}=3.98. The introduction of the HaloTag® ligand (4_{HTL}) decreased this value (logD_{7.4}=3.59), and the biotin compound (14) saw a further decrease (logD_{7.4}=3.06). Again, like JF₆₀₈ series, the higher propensity of the JF₆₄₆ unit to adopt the lipophilic lactone form (FIG. 1C) balances the polar biotin-PEG moiety in ligand 14, giving a more optimal logD_{7.4} value compared to the biotin-JF₅₄₉-HaloTag® ligand (12).

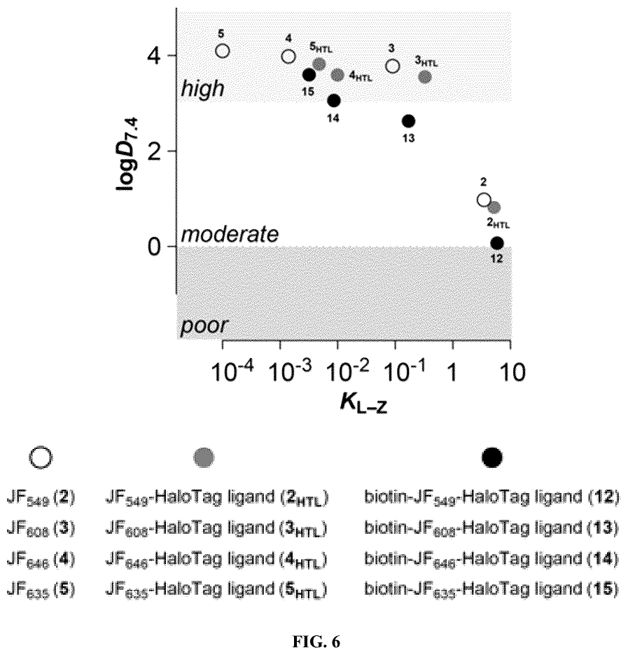

Finally, the biotin-JF₆₃₅-HaloTag® ligand (15) was tested, again comparing it to the unsubstituted JF₆₃₅-HaloTag® ligand (5_{HTL}). As expected from the properties of the parent dye (5; K_{L-Z}=0.0001), the JF₆₃₅-based HaloTag® ligands 5_{HTL} and 15 showed the lowest K_{L-Z} values of the compound series, 0.0048 and 0.0032, respectively. The distribution coefficients were correspondingly higher: the parent dye JF₆₃₅ (5) giving logD_{7.4}=4.1; the JF₆₃₅-HaloTag® ligand (5_{HTL}) showing logD_{7.4}=3.82; and the biotin-JF₆₃₅-HaloTag® ligand (15) exhibiting logD_{7.4}=3.6. These data follow the same trend as the JF₆₄₆-based 4, 4_{HTL}, and 14. Overall, the addition of polar biotin functionality via the carboxamide moiety on azetidine has relatively minor influence on K_{L-Z} values for a given dye class. The polar biotin functionality decreases the logD_{7.4} values of ligands. However, choosing low K_{L-Z} rhodamines as linkers to drive the lipophilic lactone confers biologically suitable logD_{7.4} (FIG. 6).

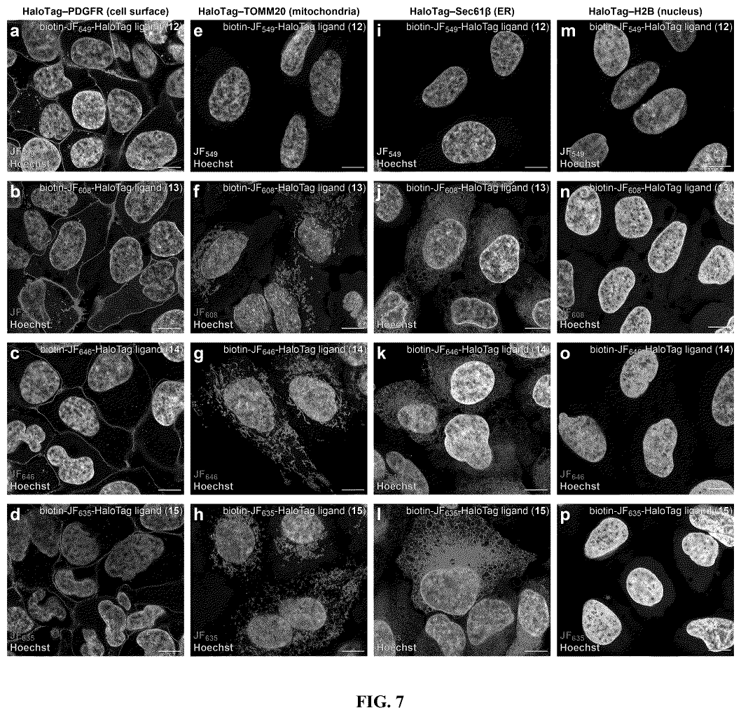

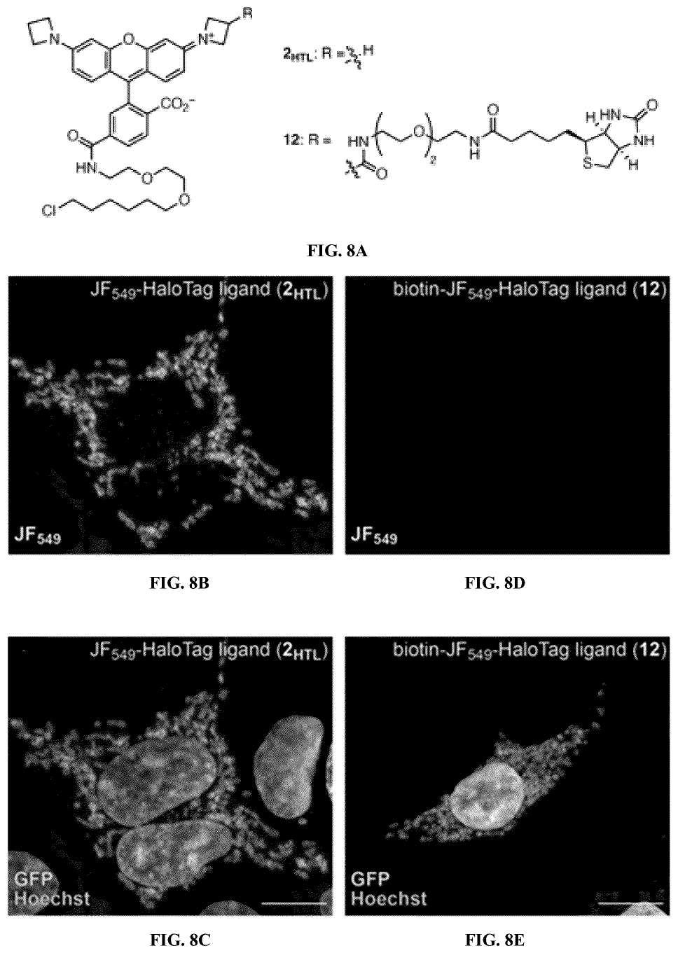

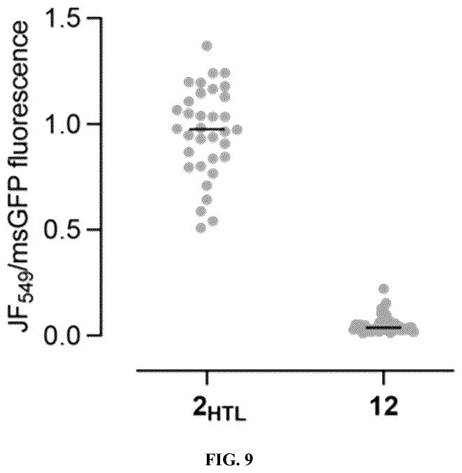

Live cell imaging with biotin-rhodamine HaloTag® ligands. Having evaluated biotin ligands 12-15 in vitro, their ability to label HaloTag® fusions in live mammalian cells was tested. U2OS cells were transiently transfected with plasmids encoding the HaloTag® protein fused to the following proteins: (1) cell surface-localized platelet-derived growth factor receptor (PDGFR); (2) outer mitochondria membrane-localized TOMM20; (3) endoplasmic reticulum (ER)-localized Sec61β; and (4) nucleus-localized histone H2B (FIG. 7, panels a-p). Incubation with live U2OS cells expressing the extracellular HaloTag®-PDGFR fusion protein resulted in robust labeling of the cellular membrane with biotin-JF₅₄₉-HaloTag® ligand (12, FIG. 7, panel a). Unlike the extracellular protein labeling, 12 did not show an appreciable signal in cells expressing the intracellular HaloTag® fusions (FIG. 7, panels e, i, m). This was confirmed further by side-by-side comparison of biotin-JF₅₄₉-HaloTag® ligand (12) and cell-permeable JF₅₄₉-HaloTag® ligand (2_{HTL}) in another cell type. As expected, the parent compound 2_{HTL} labeled the mitochondria-localized HaloTag®-msGFP fusions inside HEK293T cells at nanomolar concentration, the biotin-containing ligand 12 did not show appreciable cellular labeling measured using fluorescence microscopy (FIG. 8A-8E). Quantification of the intracellular fluorescence signal from JF₅₄₉ and msGFP channel further confirmed the poor labeling from 12 (FIG. 9). The ability of 12 to be highly fluorescent upon HaloTag® labeling but exhibiting low membrane-permeability is unsurprising given its poor logD_{7.4} (FIG. 5C) and proclivity to adopt the polar zwitterionic form (K_{L-Z}=5.9).

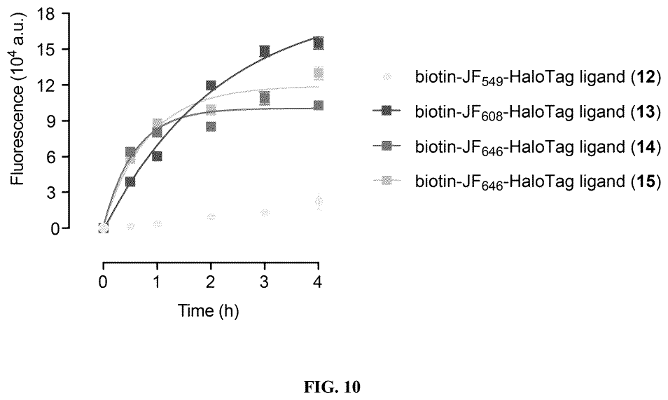

Next, carborhodamine and Si-rhodamine containing biotin ligands 13-15 that showed optimal K_{L-Z} and logD_{7.4} values were tested. Testing these ligands for labeling HaloTag® fusions at different subcellular location was also important since some rhodamine derivatives have inherent localization to specific subcellular locales.⁴¹⁻⁴³ For all three ligands, bright labeling was observed for the extracellular target (FIG. 7, panels b-d). Importantly, bright labeling was also observed for the three different intracellular targets (FIG. 7, panels f-h, j-l, n-p), confirming their high cell-permeability and HaloTag®-mediated labeling inside cells. The live cell loading kinetics of 200 nM biotin ligands 12-15 was also evaluated using U2OS cells stably expressing HaloTag®-H2B fusion protein (FIG. 10). As expected, biotin-JF₅₄₉-HaloTag® ligand (12) has negligible membrane permeability. The carborhodamine based ligand (13) reaches saturation at 4 h. The Si-rhodamine containing ligands 14 and 15 are faster than 13 and reach saturation at approximately 1 h.

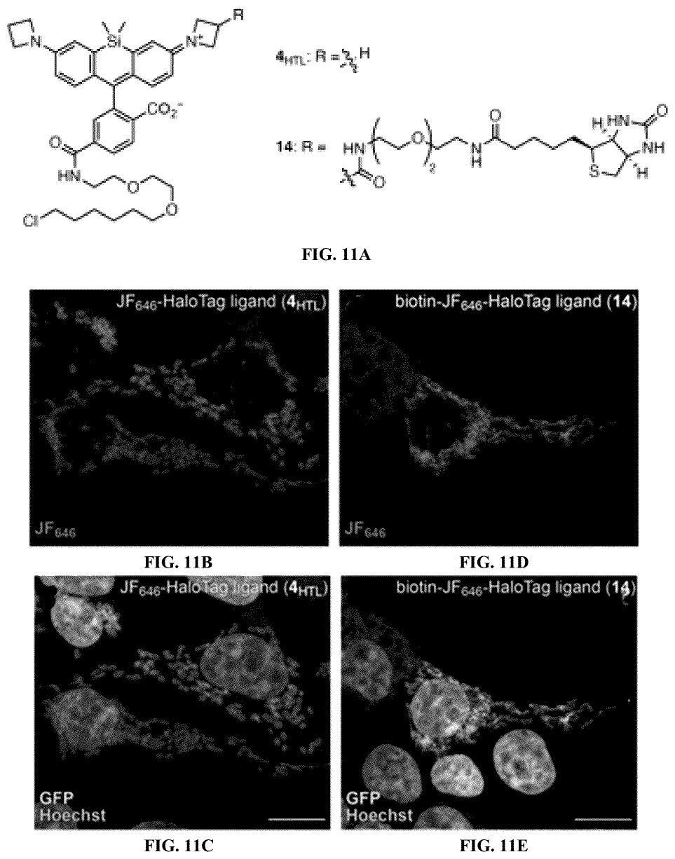

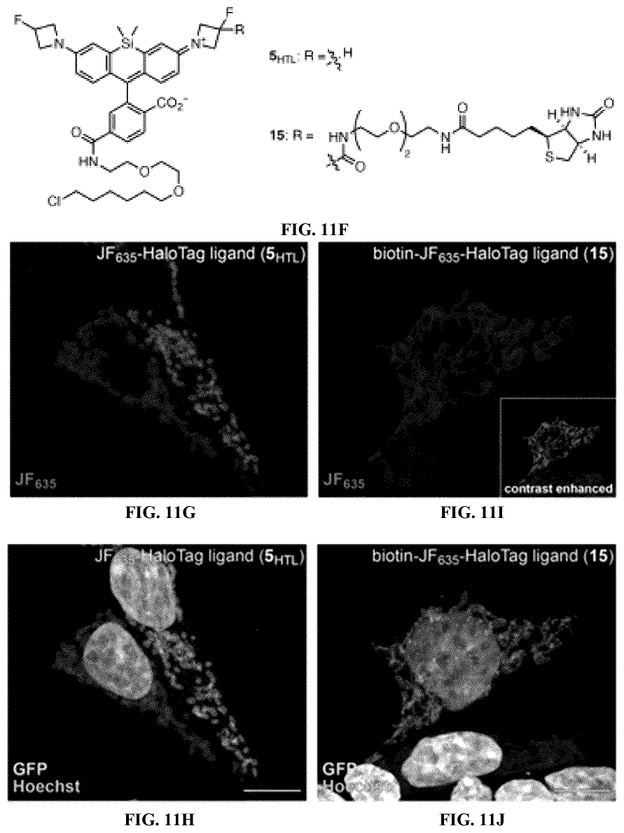

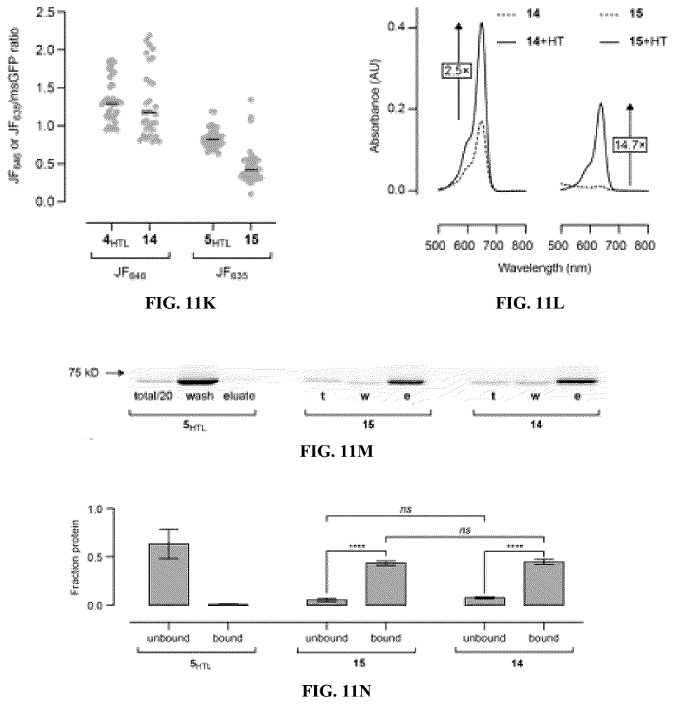

Affinity purification using biotin-rhodamine-HaloTag® ligands. The cell permeable biotin ligands were evaluated in purifying mitochondria targeted msGFP-HaloTag® fusion protein in HEK293T cells using the assay mentioned above (FIG. 3A). Si-rhodamine containing biotin-JF₆₄₆-HaloTag® ligand (14) and biotin-JF₆₃₅-HaloTag® ligand (15) were also evaluated due to their faster labeling kinetics and near infrared emission. JF₆₄₆-HaloTag® ligand (4_{HTL}) and JF₆₃₅-HaloTag® ligand (5_{HTL}) which lack biotin were also evaluated. As expected, fluorescence microscopy experiments revealed that 4_{HTL}, 5_{HTL}, 14, and 15 labeled the mitochondria in live cells at nanomolar concentrations (FIG. 11A-11J). The fluorescence signal of Si-rhodamine overlapped well with the signal from msGFP to further verify the precise labeling of HaloTag® fusion protein. Under the same imaging conditions, biotin-JF₆₃₅-HaloTag® ligand (15) showed lower intensity and required higher contrast settings (FIG. 11I). The imaging experiments using JF-HaloTag® ligands (4_{HTL} and 5_{HTL}) and biotin-JF-HaloTag® ligands (14 and 15) were repeated and quantified (FIG. 11K), showing that the two JF₆₄₆ ligands 4_{HTL} and 14 exhibit similar average intensities. For the JF₆₃₅ derived compounds, the biotin ligand 15 showed lower fluorescence intensity than 5_{HTL}; both JF₆₃₅ compounds exhibited lower intensity than the JF₆₄₆ compounds under equivalent imaging conditions. These results were consistent with the in vitro characterization of the HaloTag® conjugates (FIG. 5B). The biotin-JF₆₄₆-HaloTag® ligand (14) exhibits substantially higher absorptivity than the biotin-JF₆₃₅-HaloTag® ligand (15) but less fluorogenicity (2.5× vs 14.7×) upon conjugation to the HaloTag® protein, resulting in higher fluorescence intensity in cells (FIG. 11L).

Since the differences in cellular intensity between compounds 14 and 15 could be explained by their distinct spectral properties (FIG. 5B), and the two compounds exhibited similar logD_{7.4} values (FIG. 5C), it was expected that their performance as affinity labels would be similar. Therefore these ligands were tested by labeling, cell lysis, and capture of biotin-labeled mitochondria using magnetic streptavidin beads followed by mitochondrial lysis (FIG. 2A). As expected, the use of the control, non-biotinylated JF₆₃₅-HaloTag® ligand (5_{HTL}) gave no appreciable protein capture. Use of the biotin-JF₆₄₆-HaloTag® ligand (14), however, showed efficient labeling of protein, yielding a capture efficiency of 44.8±2.8% (mean±SEM); this was equivalent to the capture efficiency (43.4±2.3%) of the biotin-JF₆₃₅-HaloTag® ligand (15; FIG. 11M-11N). These experiments gave modest amounts of unlabeled protein in the wash; the difference between the two ligands was not significant (FIG. 11N). Overall, these experiments demonstrate that both Si-rhodamine-based biotin ligands 14 and 15 enter cells, efficiently label HaloTag® proteins, and present a biotin moiety accessible to streptavidin resins for affinity capture.

Synthesis and properties of rhodamine containing JQ1 ligands. Having

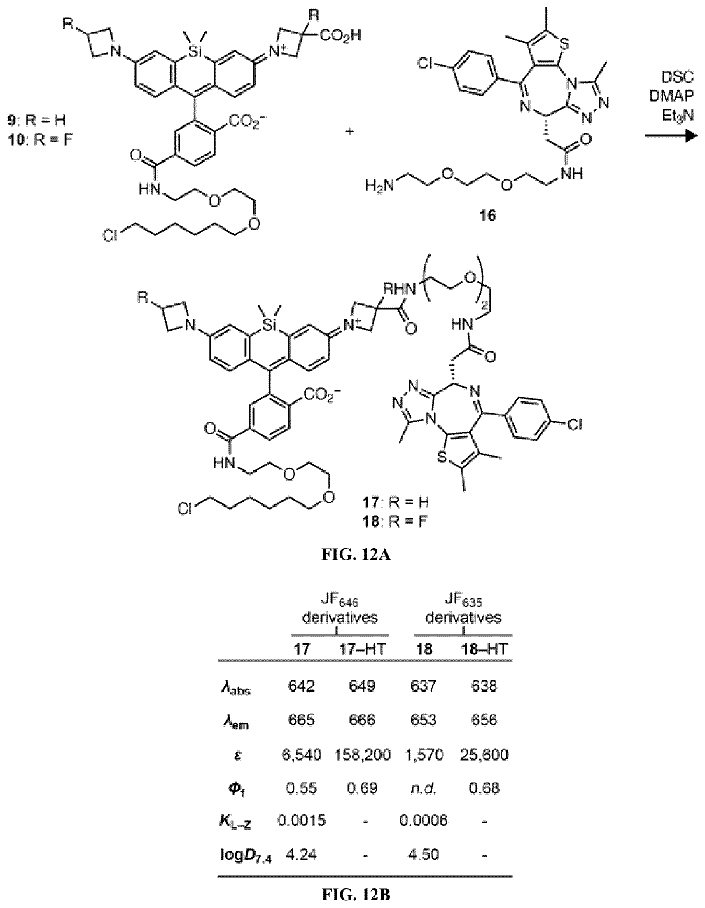

verified the ability of rhodamines to act as linkers to create cell permeable multifunctional reagents for imaging and affinity purification, their ability to act as linkers for generating multifunctional reagents for imaging and protein manipulation in living cells was tested. (+)-JQ1 ligand was used for protein manipulation. (+)-JQ1 is an inhibitor of bromodomain and extra-terminal motif (BET) family of proteins. Structural analogs of JQ1 are undergoing clinical trials for various types of diseases and BET proteins are critical target for development of drugs. The near infrared absorbing Si-rhodamine dye JF₆₄₆ and JF₆₃₅ were selected to append (+)-JQ1 due to their fluorogenic nature upon binding to HaloTag® proteins (FIG. 5B and FIG. 11L) which is advantageous when working with cells where repetitive washing is detrimental. Using the conjugating strategy from FIG. 5A, the 3″-carboxy-JF₆₄₆-HaloTag® ligand (9) and 3″-carboxy-JF₆₃₅-HaloTag® ligand (10) were added to (+)-JQ1-PEG₂-NH₂ (16; Scheme 3) to synthesize (+)-JQ1-JF₆₄₆-HaloTag® ligand (17) and (+)-JQ1-JF₆₃₅-HaloTag® ligand (18; FIG. 12A). The position of PEG₂ linker on (+)-JQ1 has been previously used and is based on the solvent exposed part in the crystal structure of (+)-JQ1 bound to BRD4.

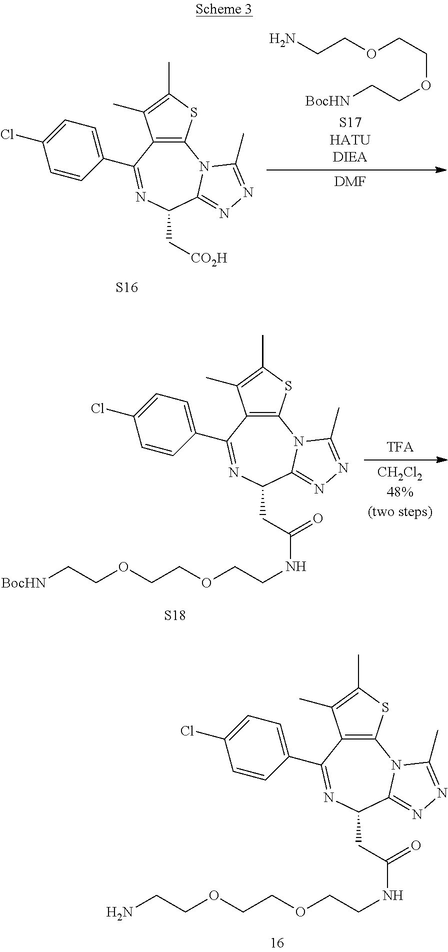

The spectral and chemical properties of JQ1 ligands were measured (FIG. 12B). The (+)-JQ1-JF₆₄₆-HaloTag® ligand (17) exhibits a slight hypsochromic shift in absorbance maxima (λ_{abs}=642 nm), has similar emission maxima (λₑₘ=665 nm), slightly higher absorptivity (ε=6,540 M⁻¹cm⁻¹), and similar fluorescence quantum yield (Φ_{f}=0.55) to the parent dye JF₆₄₆ (4; FIG. 5B). Upon binding to the HaloTag® (17-HT), the absorption shows a slight bathochromic shift with minimal change in emission maxima (λ_{abs}/λₑₘ=649 nm/666 nm) and large change in absorptivity and fluorescence quantum yield increase (ε=158,200 M⁻¹cm⁻¹, Φ_{f}=0.69). The K_{L-Z} value of 17 (K_{L-Z}=0.0015) is lower than the unsubstituted JF₆₄₆-HaloTag® ligand (4_{HTL}; K_{L-Z}=0.010) and almost same as the parent dye JF₆₄₆ (4; K_{L-Z}=0.0014). This suggests that unlike the polar biotin (clogP=−1.28), relatively non-polar JQ1 (clogP=1.79) cancels the increased polarity from the addition of 3″-carboxamide and 6-carboxamide HaloTag® ligand to shift the equilibrium back towards lipophilic form (FIG. 1C). This is further reflected in the higher logD_{7.4} of 17 (logD_{7.4}=4.24) compared to the parent dye 4 (logD_{7.4}=3.98).

The fluorinated (+)-JQ1-JF₆₃₅-HaloTag® ligand (18) shows a hypsochromic shift in spectral properties (λ_{abs}/λₑₘ=637 nm/653 nm) relative to 17. Like the parent compound, fluorination shifts the lactone-zwitterion equilibrium towards the lactone form with low absorptivity (ε=1,570 M⁻¹cm⁻¹) but with an undetectable fluorescence quantum yield. Binding to HaloTag® (18-HT) has minimal effect on spectra (λ_{abs}/λₑₘ=638 nm/656 nm) but increased absorptivity (ε=25,600 M⁻¹cm⁻¹) and quantum yield (Φ_{f}=0.68). Like 17, K_{L-Z} value of 18 (K_{L-Z}=0.0006) is lower than the unsubstituted JF₆₃₅-HaloTag® ligand (5_{HTL}; K_{L-Z}=0.0048) and similar to the parent dye JF₆₃₅ (5; K_{L-Z}=0.0001). Further, like the biotin ligands, logD_{7.4} of 18 (logD_{7.4}=4.50) is higher than 17. Overall, (+)-JQ1-JF₆₄₆-HaloTag® ligand (17) has higher brightness than (+)-JQ1-JF₆₃₅-HaloTag® ligand (18) both before and after conjugation to the HaloTag®.

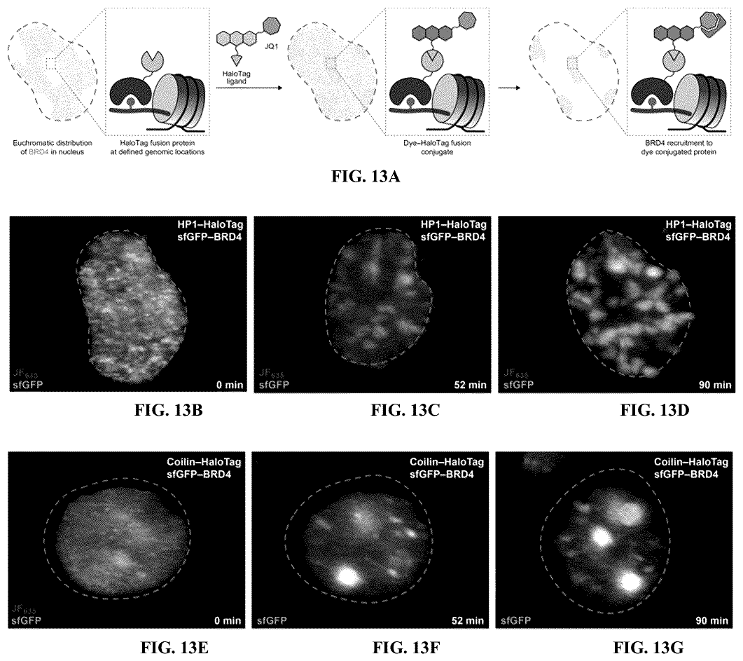

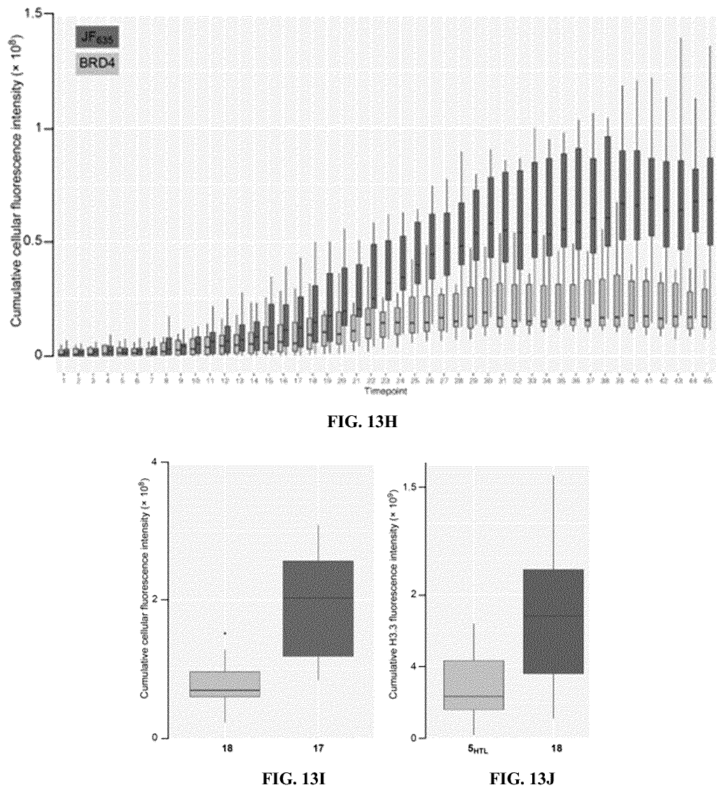

Live cell protein imaging and manipulation using JQ1-rhodamine-HaloTag® ligand. The ability of JQ1 ligands to recruit BET family protein BRD4 to HaloTag® fusion proteins at distinct genomic regions in N2a cells was then tested (FIG. 13A). N2a cells are neural progenitor cells that are frequently utilized to study neuronal differentiation and axonal growth. Hence, reagents that localize genomic proteins to specific locations in N2a cells would potentially allow testing fundamental questions about neuronal development. N2a cells were transiently transfected with plasmids encoding the HaloTag® protein fused to the following nuclear proteins: (1) constitutive heterochromatin marker heterochromatin protein 1 (HP1); (2) Cajal body component coilin; and (3) centromere localized histone H3 variant centromere protein A (CENP-A). These three proteins have a very distinct distribution in the nucleus than BRD4 which generally has an euchromatic distribution. Localization of BRD4 to specific gene loci can induce gene expression. Hence, cell permeable small molecule ligands with robust BRD4 recruitment could be useful tools for manipulating gene expression. To independently visualize BRD4, superfolder GFP-BRD4 fusion was created, and to discern the nuclear boundary, histone 3.3-SNAP was used and labeled it with dye-SNAP tag®.

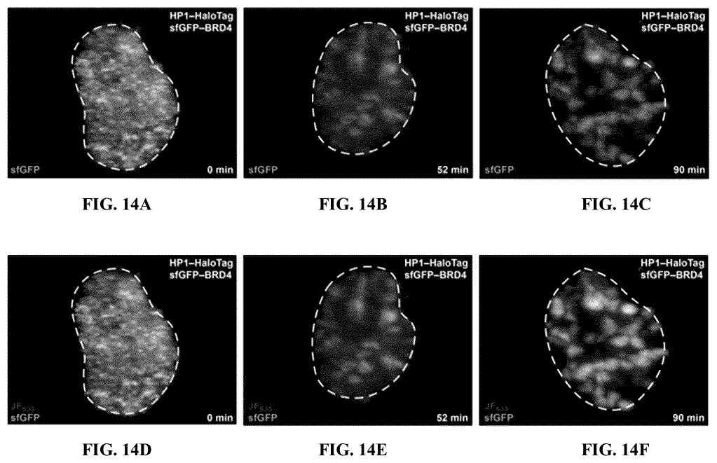

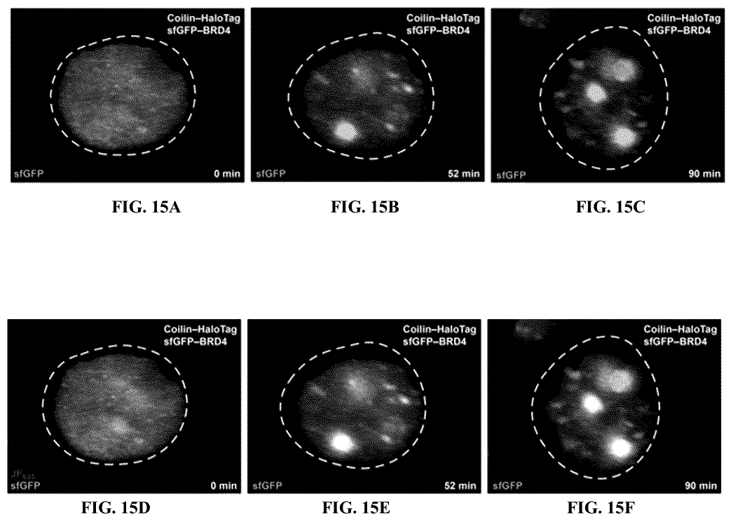

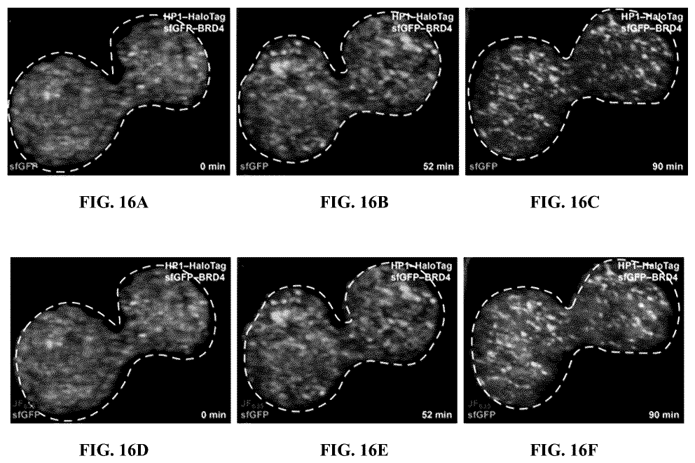

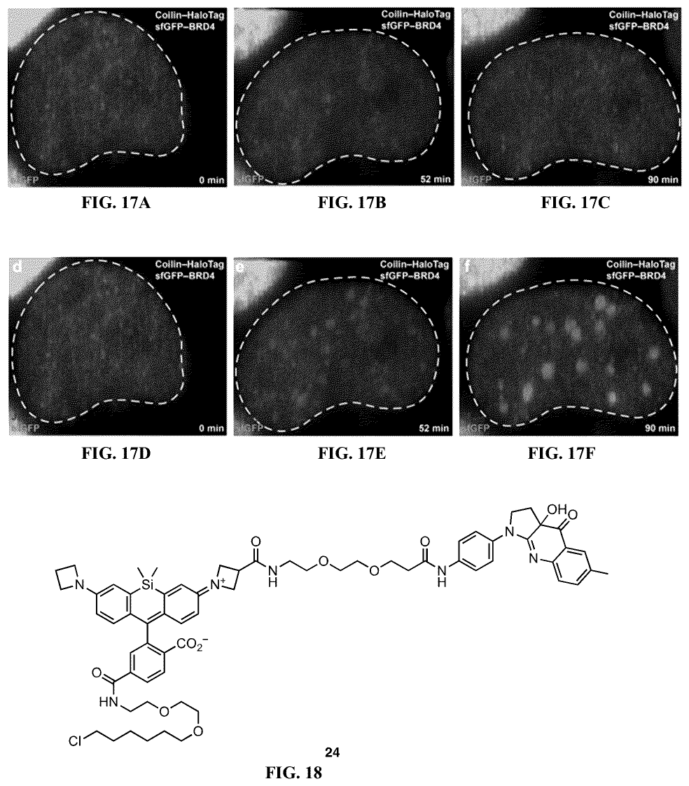

Initially, a side-by-side comparison of JF₆₄₆ based 17 and JF₆₃₅ based 18 was performed for recruiting BRD4 to coilin-HaloTag® under a “no wash” lattice lightsheet fluorescence microscopy setup. Both the reagents were successful in recruiting BRD4 to coilin-HaloTag®. Quantification of imaging data showed 17 exhibited relatively brighter labeling (FIG. 13I) and 18 has more robust BRD4 recruitment, in accordance with the in vitro brightness, K_{L-Z}, and logD_{7.4} data (FIG. 12B). So, ligand 18 was selected for further experiments. Advantageously, 18 also has lower absorptivity and negligible fluorescence quantum yield before HaloTag® conjugation. “No wash” imaging was performed and the fluorescence from JF₆₃₅ and sfGFP were monitored. Labeling of HP1 with 18 altered the uniform euchromatic distribution of BRD4 across nucleus and localized it to HP1 in a robust and time dependent manner (FIG. 13B-13D, FIG. 14A-14F). Labeling of coilin with 18 also robustly localized BRD4 to coilin (FIG. 13E-13G, FIG. 15A-15F) in a time dependent manner. It was also confirmed that the specificity of BRD4 localization was (+)-JQ1 dependent by using (−)-JQ1-JF₆₃₅-HaloTag® ligand (19; Scheme 4). As expected, incubation of N2a cells with 19 did not result in BRD4 localization to HP1 (FIG. 16A-16F) or coilin (FIG. 17A-17F). The BRD4 recruitment to coilin-HaloTag® across multiple cells was quantified to show that BRD4 is indeed recruited to coilin labeled with 18 (FIG. 13H). Recruitment of BRD4 to HP1 (marker for constitutive chromatin) is expected to increase the transcription at HP1. Histone H3 variant H3.3 typically accumulates in transcriptionally active nucleosomes and are usually not present in constitutive heterochromatin. The H3.3at HP1-HaloTag® labeled with either JF₆₃₅-HaloTag® ligand (5_{HTL}) or the JQ1 analog 18 were quantified (FIG. 13J). JQ1 labeled HP1 has significantly more H3.3 to show that BRD4 recruited into a transcriptionally repressed domain can increase transcription.

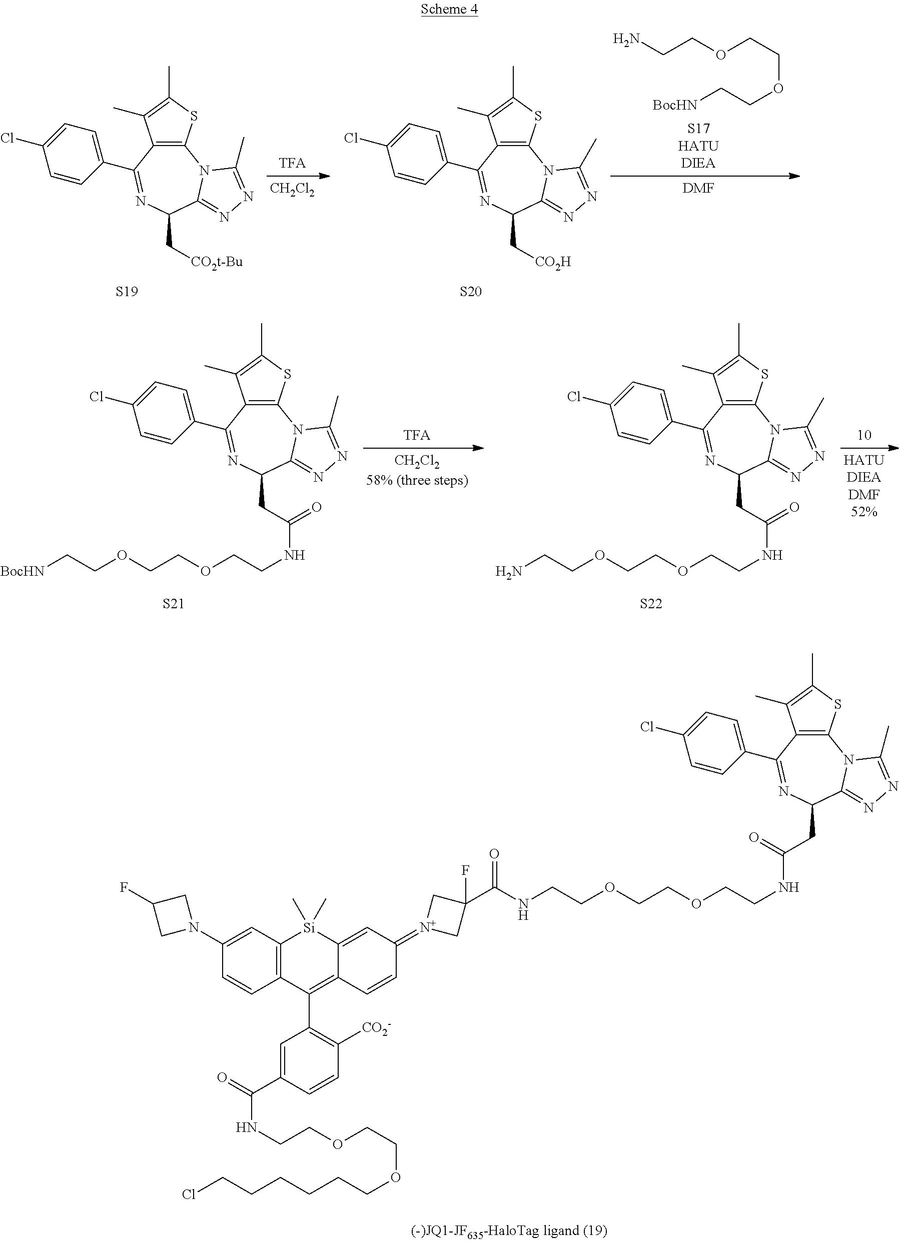

The ability to specifically label proteins with synthetic small-molecules inside living cells is a powerful technique for biology.¹⁻³ The generality of these systems has led to the development of numerous ligands to probe, perturb, or purify cellular components. Depending on the specific makeup of functional small-molecule, linker, and ligand, some compounds, such as biotin-containing HaloTag® ligands, suffer from poor performance or low cell-permeability (FIG. 3A-3D, FIG. 4A-4D). These issues were addressed by designing compounds where a rhodamine dye serves as the linker in-between the self-labeling tag ligand and the biotin affinity reagent. Four ligands were synthesized with different rhodamine dye linkers that exhibit high and low K_{L-Z} values, and change the logD_{7.4} of the overall ligand (FIG. 5A-5C, FIG. 6). Biotin-JF₅₄₉-HaloTag® ligand (12) exhibited poor cell-permeability (FIG. 7, panels a-p, FIG. 8A-8E). This is due to a combination of the biotin moiety increasing the propensity of the dye linker to adopt the zwitterionic form (i.e., high K_{L-Z}) and the polar nature of the biotin functionality itself, which substantially decreases logD_{7.4}. In contrast, the ligands containing JF₆₀₈, JF₆₄₆ and JF₆₃₅ (13-15) readily entered cells (FIG. 10) to label HaloTag® fusions at different subcellular locations (FIG. 7, panels a-p) and yielded a biotin conjugate that showed efficient affinity capture (FIG. 11A-11N). This improved performance is due to the higher logD_{7.4} values, which stem from the lower lactone-zwitterion equilibrium constants (K_{L-Z}) exhibited by the carborhodamine and Si-rhodamine linkers. This approach was extended to add the non-polar pharmacophore JQ1 to the fluorogenic Si-rhodamine dyes (FIG. 12A-12B) for translocation of BRD4 to three distinct nuclear regions (FIG. 13A-13J). These compounds would enable new experiments to isolate proteins/organelles and manipulate proteins in HaloTag®-expressing cells.

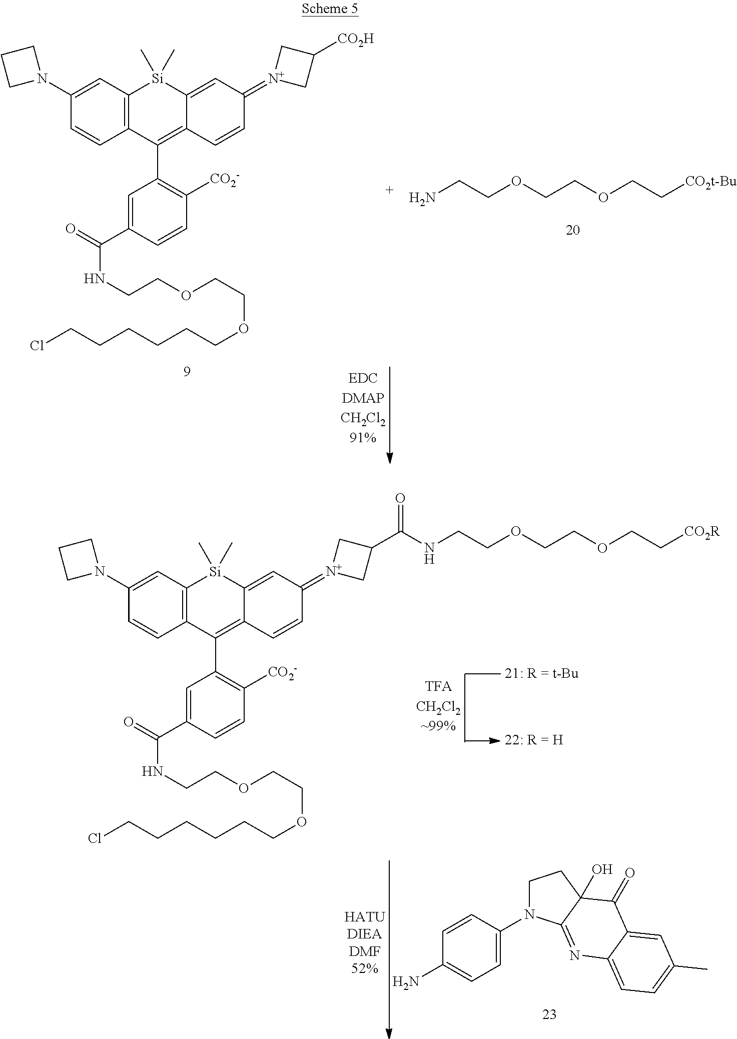

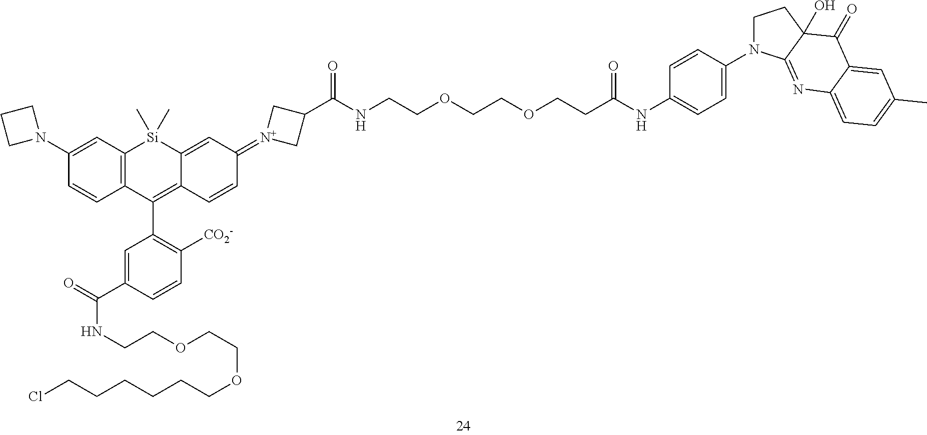

Incorporating a rhodamine linker into a molecule is not without cost. Despite the advances in fluorophore chemistry over the past decade,^{13, 43-44} the synthesis of rhodamines still requires multiple steps. The dye moiety must then be incorporated into the final multifunctional molecule adding size and complexity. Nevertheless, this compromise in atom economy is warranted after considering the performance of compounds 14 and 15 compared to the simpler and smaller biotin-HaloTag® (6). Compound 6 shows no appreciable affinity capture (FIG. 3A-3D) and requires high concentrations to achieve cellular labeling (10 μM; FIG. 4A-4D). Compounds 14 and 15 enable efficient affinity capture due to the Si-rhodamine linker, and can be deployed at 100-fold lower concentration (100 nM; FIG. 7, panels a-p, FIG. 11A-11N). This increase in efficacy and potency ameliorates the increased effort required to synthesize these Si-rhodamine-linked compounds. These labeling conditions match those used for unfunctionalized ligands, suggesting that the biotin-appended compounds 13-15 can be used without drastic changes to labeling protocols. In addition to the improved performance, the incorporation of a fluorescent linker also allows confirmation of the biotin labeling in cells using fluorescent microscopy and facile visualization of protein in SDS-PAGE gels. The concentration of probes for maximal pulldown will need to be optimized for a given protein, though 100-250 nM is suggested as a good starting point. The biotin ligands described herein also exhibit distinct advantages over the commercial HaloLink resins for affinity pulldown of HaloTag® fusion proteins. The ligands disclosed herein allow (1) pulse-chase labeling of proteins in living cells, (2) visualization of spatial distribution of labeled proteins, (3) isolation of whole organelles without rupturing them (mitochondria were isolated, for example, as disclosed herein), and (4) affinity purification in mammalian systems. HaloLink has worked well in bacterial systems but shows poor performance in mammalian systems. Further, the cell-impermeable biotin-JF₅₄₉-HaloTag® ligand, in combination with cell permeable biotin ligands, opens the possibility of visualizing and quantifying subpopulations of cell-surface and cytosolic proteins (e.g., receptors). Work along this direction is currently underway. Given that biotin ligands disclosed herein label proteins in distinct subcellular locations and affinity purify overexpressed proteins, it would be expected these probes to be compatible with HaloTag® fusions of proteins expressed at endogenous levels. However, affinity purification of overexpressed proteins itself is tremendously useful for cell biology and biochemistry experiments. If working with endogenously expressed proteins, amount of probe could be further decreased to boost the atom economy. It is also noted that careful attention must be paid to endogenously biotinylated proteins when working with and streptavidin-biotin pulldown protocols.

Looking forward, this strategy of “rhodamine-as-linker” should permit the creation of other useful tools for cell biology. This was demonstrated for the pharmacophore JQ1 for recruiting proteins at defined nuclear locations (FIG. 13A-13J) and can be extended to other molecules such as blebbistatin, which was incorporated into blebbistatin-JF₆₄₆-HaloTag® ligand (24; Scheme 5, FIG. 18). The work establishes that adjusting the K_{L-Z} of the dye component is sufficient to improve the cell permeability of a multifunctional fluorophore. Both the biotin and JQ1-containing ligands follow the K_{L-Z} trends of the parent JF₅₄₉, JF₆₀₈, JF₆₄₆, and JF₆₃₅ dyes;²³ the K_{L-Z} values are inversely proportional to logD_{7.4}. This suggests that future work can leverage the extensive development of dye-based ligands and known structure-activity relationships of small-molecule fluorophores.^{13, 24-26} Exchanging the biotin or JQ1 moiety with other chemical entities (e.g., reactive groups, pharmacological agents), followed by fine-tuning of the ligand properties using different rhodamine dye linkers (e.g., fluorinated rhodamines^{13,24}) is contemplated to provide a useful toolbox of probes that enable sophisticated biological experiments in living cells.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Xue, L.; Karpenko, I. A.; Hiblot, J.; Johnsson, K., Imaging and manipulating proteins in live cells through covalent labeling. Nat. Chem. Biol. 2015, 11 (12), 917-923.
2. Lavis, L. D., Chemistry is dead. Long live chemistry! Biochemistry 2017, 56 (39), 5165-5170.
3. Kumar, P.; Lavis, L. D., Melding synthetic molecules and genetically encoded proteins to forge new tools for neuroscience. Annu. Rev. Neurosci. 2022, 45, 131-150.
4. Los, G. V.; Encell, L. P.; McDougall, M. G.; Hartzell, D. D.; Karassina, N.; Zimprich, C.; Wood, M. G.; Learish, R.; Ohana, R. F.; Urh, M., HaloTag: A novel protein labeling technology for cell imaging and protein analysis. ACS Chem. Biol. 2008, 3 (6), 373-382.
5. Keppler, A.; Gendreizig, S.; Gronemeyer, T.; Pick, H.; Vogel, H.; Johnsson, K., A general method for the covalent labeling of fusion proteins with small molecules in vivo. Nat. Biotechnol. 2003, 21 (1), 86-89.
6. England, C. G.; Luo, H.; Cai, W., HaloTag technology: A versatile platform for biomedical applications. Bioconjug. Chem. 2015, 26 (6), 975-986.
7. Ohana, R. F.; Kirkland, T. A.; Woodroofe, C. C.; Levin, S.; Uyeda, H. T.; Otto, P.; Hurst, R.; Robers, M. B.; Zimmerman, K.; Encell, L. P.; Wood, K. V., Deciphering the cellular targets of bioactive compounds using a chloroalkane capture tag. ACS Chem. Biol. 2015, 10 (10), 2316-2324.
8. Murrey, H. E.; Judkins, J. C.; Am Ende, C. W.; Ballard, T. E.; Fang, Y.; Riccardi, K.; Di, L.; Guilmette, E. R.; Schwartz, J. W.; Fox, J. M.; Johnson, D. S., Systematic evaluation of bioorthogonal reactions in live cells with clickable HaloTag ligands: Implications for intracellular imaging. J. Am. Chem. Soc. 2015, 137 (35), 11461-11475.
9. Peraro, L.; Deprey, K. L.; Moser, M. K.; Zou, Z.; Ball, H. L.; Levine, B.; Kritzer, J. A., Cell penetration profiling using the chloroalkane penetration assay. J. Am. Chem. Soc. 2018, 140 (36), 11360-11369.
10. Hoelzel, C. A.; Zhang, X., Visualizing and manipulating biological processes by using HaloTag and SNAP-Tag technologies. ChemBioChem 2020, 21 (14), 1935-1946.
11. Deo, C.; Abdelfattah, A. S.; Bhargava, H. K.; Berro, A. J.; Falco, N.; Farrants, H.; Moeyaert, B.; Chupanova, M.; Lavis, L. D.; Schreiter, E. R., The HaloTag as a general scaffold for far-red tunable chemigenetic indicators. Nat. Chem. Biol. 2021, 17 (6), 718-723.
12. Grimm, J. B.; Xie, L.; Casler, J. C.; Patel, R.; Tkachuk, A. N.; Falco, N.; Choi, H.; Lippincott-Schwartz, J.; Brown, T. A.; Glick, B. S.; Liu, Z.; Lavis, L. D., A general method to improve fluorophores using deuterated auxochromes. JACS Au 2021, 1 (5), 690-696.
13. Grimm, J. B.; Tkachuk, A. N.; Xie, L.; Choi, H.; Mohar, B.; Falco, N.; Schaefer, K.; Patel, R.; Zheng, Q.; Liu, Z.; Lippincott-Schwartz, J.; Brown, T. A.; Lavis, L. D., A general method to optimize and functionalize red-shifted rhodamine dyes. Nat. Methods 2020, 17 (8), 815-821.
14. Deo, C.; Sheu, S. H.; Seo, J.; Clapham, D. E.; Lavis, L. D., Isomeric tuning yields bright and targetable red Ca(2+) indicators. J. Am. Chem. Soc. 2019, 141 (35), 13734-13738.
15. Abdelfattah, A. S.; Kawashima, T.; Singh, A.; Novak, O.; Liu, H.; Shuai, Y.; Huang, Y. C.; Campagnola, L.; Seeman, S. C.; Yu, J.; Zheng, J.; Grimm, J. B.; Patel, R.; Friedrich, J.; Mensh, B. D.; Paninski, L.; Macklin, J. J.; Murphy, G. J.; Podgorski, K.; Lin, B. J.; Chen, T. W.; Turner, G. C.; Liu, Z.; Koyama, M.; Svoboda, K.; Ahrens, M. B.; Lavis, L. D.; Schreiter, E. R., Bright and photostable chemigenetic indicators for extended in vivo voltage imaging. Science 2019, 365 (6454), 699-704.
16. Mertes, N.; Busch, M.; Huppertz, M. C.; Hacker, C. N.; Wilhelm, J.; Gurth, C. M.; Kuhn, S.; Hiblot, J.; Koch, B.; Johnsson, K., Fluorescent and bioluminescent calcium indicators with tuneable colors and affinities. J. Am. Chem. Soc. 2022, 144 (15), 6928-6935.
17. Wilhelm, J.; Kuhn, S.; Tarnawski, M.; Gotthard, G.; Tunnermann, J.; Tanzer, T.; Karpenko, J.; Mertes, N.; Xue, L.; Uhrig, U.; Reinstein, J.; Hiblot, J.; Johnsson, K., Kinetic and structural characterization of the self-labeling protein tags HaloTag7, SNAP-tag, and CLIP-tag. Biochemistry 2021, 60 (33), 2560-2575.
18. Hong, H.; Benink, H. A.; Uyeda, H. T.; Valdovinos, H. F.; Zhang, Y.; Meisenheimer, P.; Barnhart, T. E.; Fan, F.; Cai, W., HaloTag as a reporter gene: Positron emission tomography imaging with (64)Cu-labeled second generation HaloTag ligands. Am. J. Transl. Res. 2013, 5 (3), 291-302.
19. Farrants, H.; Hiblot, J.; Griss, R.; Johnsson, K., Rational design and applications of semisynthetic modular biosensors: SNIFITs and LUCIDs. Methods Mol. Biol. 2017, 1596, 101-117.
20. Deal, P. E.; Liu, P.; Al-Abdullatif, S. H.; Muller, V. R.; Shamardani, K.; Adesnik, H.; Miller, E. W., Covalently tethered rhodamine voltage reporters for high speed functional imaging in brain tissue. J. Am. Chem. Soc. 2020, 142 (1), 614-622.
21. Xue, L.; Schnacke, P.; Frei, M. S.; Koch, B.; Hiblot, J.; Wombacher, R.; Fabritz, S.; Johnsson, K., Probing coenzyme A homeostasis with semisynthetic biosensors. Nat. Chem. Biol. 2022, 19, 346-355.
22. Sallin, O.; Reymond, L.; Gondrand, C.; Raith, F.; Koch, B.; Johnsson, K., Semisynthetic biosensors for mapping cellular concentrations of nicotinamide adenine dinucleotides. Elife 2018, 7, e32638.
23. Grimm, J. B.; Muthusamy, A. K.; Liang, Y.; Brown, T. A.; Lemon, W. C.; Patel, R.; Lu, R.; Macklin, J. J.; Keller, P. J.; Ji, N.; Lavis, L. D., A general method to fine-tune fluorophores for live-cell and in vivo imaging. Nat. Methods 2017, 14 (10), 987-994.
24. Zheng, Q.; Ayala, A. X.; Chung, I.; Weigel, A. V.; Ranjan, A.; Falco, N.; Grimm, J. B.; Tkachuk, A. N.; Wu, C.; Lippincott-Schwartz, J.; Singer, R. H.; Lavis, L. D., Rational design of fluorogenic and spontaneously blinking labels for super-resolution imaging. ACS Cent. Sci. 2019, 5 (9), 1602-1613.
25. Wang, L.; Tran, M.; D'Este, E.; Roberti, J.; Koch, B.; Xue, L.; Johnsson, K., A general strategy to develop cell permeable and fluorogenic probes for multicolour nanoscopy. Nat. Chem. 2020, 12 (2), 165-172.
26. Bucevicius, J.; Kostiuk, G.; Gerasimaite, R.; Gilat, T.; Lukinavicius, G., Enhancing the biocompatibility of rhodamine fluorescent probes by a neighbouring group effect. Chem. Sci. 2020, 11 (28), 7313-7323.
27. Heynck, L.; Matthias, J.; Bossi, M. L.; Butkevich, A. N.; Hell, S. W., N-Cyanorhodamines: cell-permeant, photostable and bathochromically shifted analogues of fluoresceins. Chem. Sci. 2022, 13 (28), 8297-8306.
28. Nemoto, Y.; De Camilli, P., Recruitment of an alternatively spliced form of synaptojanin 2 to mitochondria by the interaction with the PDZ domain of a mitochondrial outer membrane protein. EMBO J. 1999, 18 (11), 2991-3006.
29. Vevea, J. D.; Chapman, E. R., Acute disruption of the synaptic vesicle membrane protein synaptotagmin 1 using knockoff in mouse hippocampal neurons. Elife 2020, 9, e56469.
30. Lisse, D.; Wilkens, V.; You, C.; Busch, K.; Piehler, J., Selective targeting of fluorescent nanoparticles to proteins inside live cells. Angew. Chem. Int. Ed. Engl. 2011, 50 (40), 9352-9355.
31. Svendsen, S.; Zimprich, C.; McDougall, M. G.; Klaubert, D. H.; Los, G. V., Spatial separation and bidirectional trafficking of proteins using a multi-functional reporter. BMC Cell Biol. 2008, 9, 17.
32. Grimm, J. B.; English, B. P.; Chen, J.; Slaughter, J. P.; Zhang, Z.; Revyakin, A.; Patel, R.; Macklin, J. J.; Normanno, D.; Singer, R. H.; Lionnet, T.; Lavis, L. D., A general method to improve fluorophores for live-cell and single-molecule microscopy. Nat. Methods 2015, 12 (3), 244-250.
33. Fu, M.; Xiao, Y.; Qian, X.; Zhao, D.; Xu, Y., A design concept of long-wavelength fluorescent analogs of rhodamine dyes: replacement of oxygen with silicon atom. Chem. Commun. 2008, (15), 1780-1782.
34. Koide, Y.; Urano, Y.; Hanaoka, K.; Terai, T.; Nagano, T., Evolution of group 14 rhodamines as platforms for near-infrared fluorescence probes utilizing photoinduced electron transfer. ACS Chem. Biol. 2011, 6 (6), 600-608.
35. Lukinavičius, G.; Umezawa, K.; Olivier, N.; Honigmann, A.; Yang, G.; Plass, T.; Mueller, V.; Reymond, L.; Corrêa Jr, I. R.; Luo, Z. G.; Schultz, C.; Lemke, E. A.; Heppenstall, P.; Eggeling, C.; Manley, S.; Johnsson, K., A near-infrared fluorophore for live-cell super-resolution microscopy of cellular proteins. Nature Chem. 2013, 5, 132-139.
36. Andres, A.; Roses, M.; Rafols, C.; Bosch, E.; Espinosa, S.; Segarra, V.; Huerta, J. M., Setup and validation of shake-flask procedures for the determination of partition coefficients (logD) from low drug amounts. Eur. J. Pharm. Sci. 2015, 76, 181-191.
37. Martin, Y. C., A bioavailability score. J. Med. Chem. 2005, 48 (9), 3164-3170.
38. Tinworth, C. P.; Young, R. J., Facts, patterns, and principles in drug discovery: Appraising the Rule of 5 with measured physicochemical data. J. Med. Chem. 2020, 63 (18), 10091-10108.
39. Waring, M. J., Lipophilicity in drug discovery. Expert Opin. Drug Discov. 2010, 5 (3), 235-248.
40. Waring, M. J., Defining optimum lipophilicity and molecular weight ranges for drug candidates-Molecular weight dependent lower logD limits based on permeability. Bioorg. Med. Chem. Lett. 2009, 19 (10), 2844-2851.
41. Johnson, L. V.; Walsh, M. L.; Chen, L. B., Localization of mitochondria in living cells with rhodamine 123. Proc. Natl. Acad. Sci. U. S. A. 1980, 77 (2), 990-994.
42. Kim, Y. K.; Lee, J. S.; Bi, X.; Ha, H. H.; Ng, S. H.; Ahn, Y. H.; Lee, J. J.; Wagner, B. K.; Clemons, P. A.; Chang, Y. T., The binding of fluorophores to proteins depends on the cellular environment. Angew. Chem. Int. Ed. Engl. 2011, 50 (12), 2761-2763.
43. Grimm, J. B.; Brown, T. A.; Tkachuk, A. N.; Lavis, L. D., General synthetic method for Si-fluoresceins and Si-rhodamines. ACS Cent. Sci. 2017, 3 (9), 975-985.
44. Dwight, S. J.; Levin, S., Scalable regioselective synthesis of rhodamine dyes. Org. Lett. 2016, 18 (20), 5316-5319.
45. Encell, L. P.; Friedman Ohana, R.; Zimmerman, K.; Otto, P.; Vidugiris, G.; Wood, M. G.; Los, G. V.; McDougall, M. G.; Zimprich, C.; Karassina, N.; Learish, R. D.; Hurst, R.; Hartnett, J.; Wheeler, S.; Stecha, P.; English, J.; Zhao, K.; Mendez, J.; Benink, H. A.; Murphy, N.; Daniels, D. L.; Slater, M. R.; Urh, M.; Darzins, A.; Klaubert, D. H.; Bulleit, R. F.; Wood, K. V., Development of a dehalogenase-based protein fusion tag capable of rapid, selective and covalent attachment to customizable ligands. Curr. Chem. Genomics 2012, 6, 55-71.
46. Suzuki, K.; Kobayashi, A.; Kaneko, S.; Takehira, K.; Yoshihara, T.; Ishida, H.; Shiina, Y.; Oishi, S.; Tobita, S., Reevaluation of absolute luminescence quantum yields of standard solutions using a spectrometer with an integrating sphere and a back-thinned CCD detector. Phys. Chem. Chem. Phys. 2009, 11 (42), 9850-9860.
47. Schönsee, C. D.; Bucheli, T., Experimental determination of octanol-water partition coefficients of selected natural toxins. J. Chem. Eng. Data 2020, 65 (4), 1946-1953.
48. Lois, C.; Hong, E. J.; Pease, S.; Brown, E. J.; Baltimore, D., Germline transmission and tissue-specific expression of transgenes delivered by lentiviral vectors. Science 2002, 295 (5556), 868-72.
49. Jordan, M.; Wurm, F., Transfection of adherent and suspended cells by calcium phosphate. Methods 2004, 33 (2), 136-143.
50. Promega Technical Note #9PIG859. www.promega.com/resources/protocols/product-information-sheets/g/HaloTag-pegbiotin-ligand-protocol; accessed Feb 1, 2023.
51. Corcelli, A.; Saponetti, M. S.; Zaccagnino, P.; Lopalco, P.; Mastrodonato, M.; Liquori, G. E.; Lorusso, M., Mitochondria isolated in nearly isotonic KCl buffer: Focus on cardiolipin and organelle morphology. Biochim. Biophys. Acta 2010, 1798 (3), 681-687.
52. Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; Tinevez, J. Y.; White, D. J.; Hartenstein, V.; Eliceiri, K.; Tomancak, P.; Cardona, A., Fiji: An open-source platform for biological-image analysis. Nat. Methods 2012, 9 (7), 676-82.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound, according to the formula: 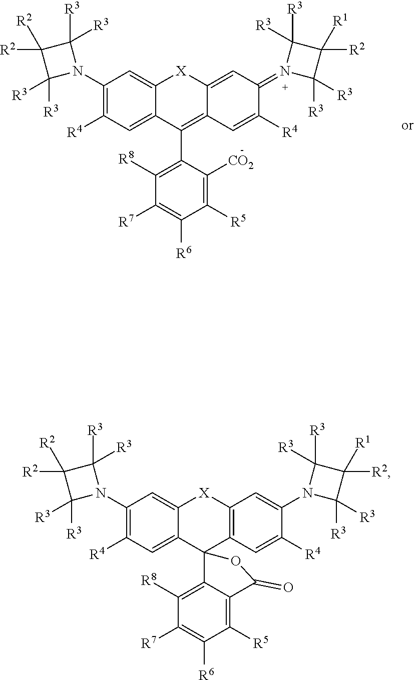
or a salt thereof, wherein:
X is O, C(CH₃)₂, or Si(CH₃)₂;
R¹ is an affinity tag-containing moiety or a protein-manipulation moiety;
each R² is independently selected from the group consisting of H, D, halogen, OH, O(alkyl), N(alkyl)₂, CF₃, CN, COOH, COO(alkyl), C(O)NH(alkyl), C(O)N(alkyl)₂, and SO₂CH₃;
each R³ is independently selected from the group consisting of H and D;
each R⁴ is independently selected from the group consisting of H, halogen, CF₃, and CN; and
R⁵, R⁶, R⁷, and R⁸ are each independently selected from the group consisting of H, F, CO₂H, and a self-labeling protein (SLP) ligand, so long as one of R⁵, R⁶, R⁷, and R⁸ is the SLP ligand.

2. The compound of claim 1, wherein R¹ is

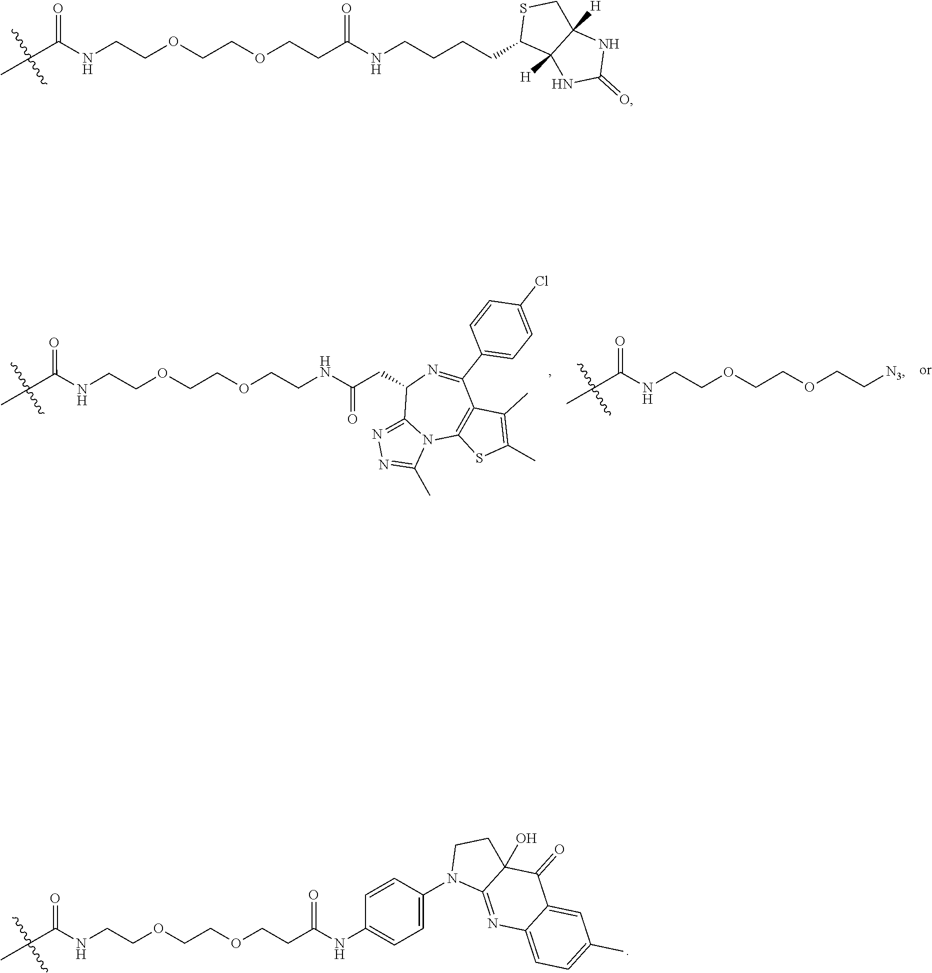

3. The compound of claim 2, wherein the SLP ligand is

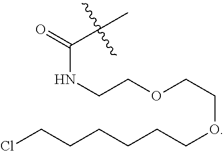 [SMILES: *C(=O)NCCOCCOCCCCCCCl]

4. The compound of claim 1, wherein the SLP ligand is

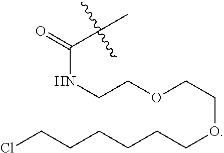 [SMILES: *C(=O)NCCOCCOCCCCCCCl]

5. The compound of claim 2, wherein R² is H or F; R³ and R⁴ are H; R⁵, R⁶, and R⁸ are H; and R⁷ is the SLP ligand.

6. The compound of claim 5, wherein X is O, Si(CH₃)₂, or Si(CH₃)₂.

7. The compound of claim 1, wherein X is O, Si(CH₃)₂, or Si(CH₃)₂.

8. The compound of claim 5, wherein the SLP ligand is

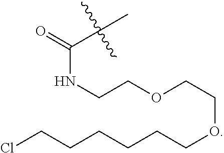 [SMILES: *C(=O)NCCOCCOCCCCCCCl]

9. The compound of claim 1, of the following formula: 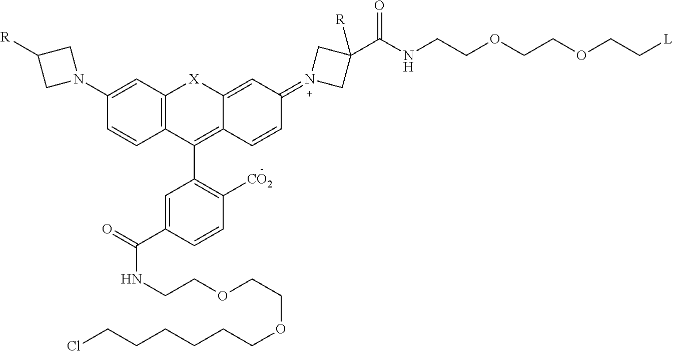
wherein R is H or F; X is O, C(CH₃)₂, or Si(CH₃)₂; and L is an affinity tag-containing moiety or a protein-manipulation moiety.

10. The compound of claim 1, of the following formula: 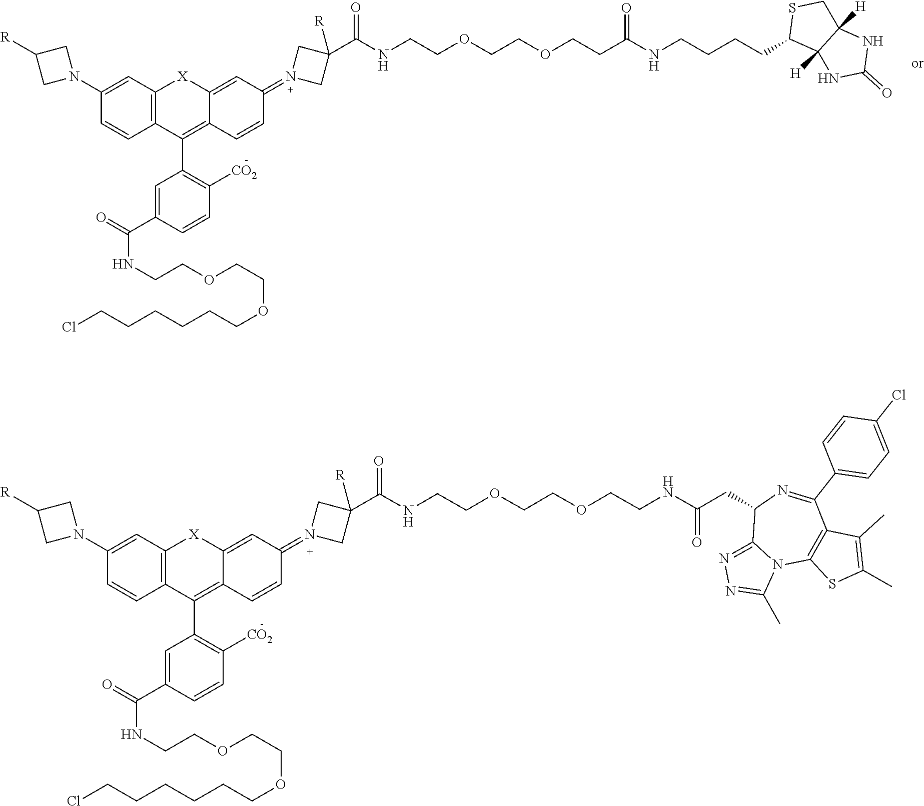
wherein R is H or F, and X is O, C(CH₃)₂, or Si(CH₃)₂.

11. The compound of claim 1, of the following formula:

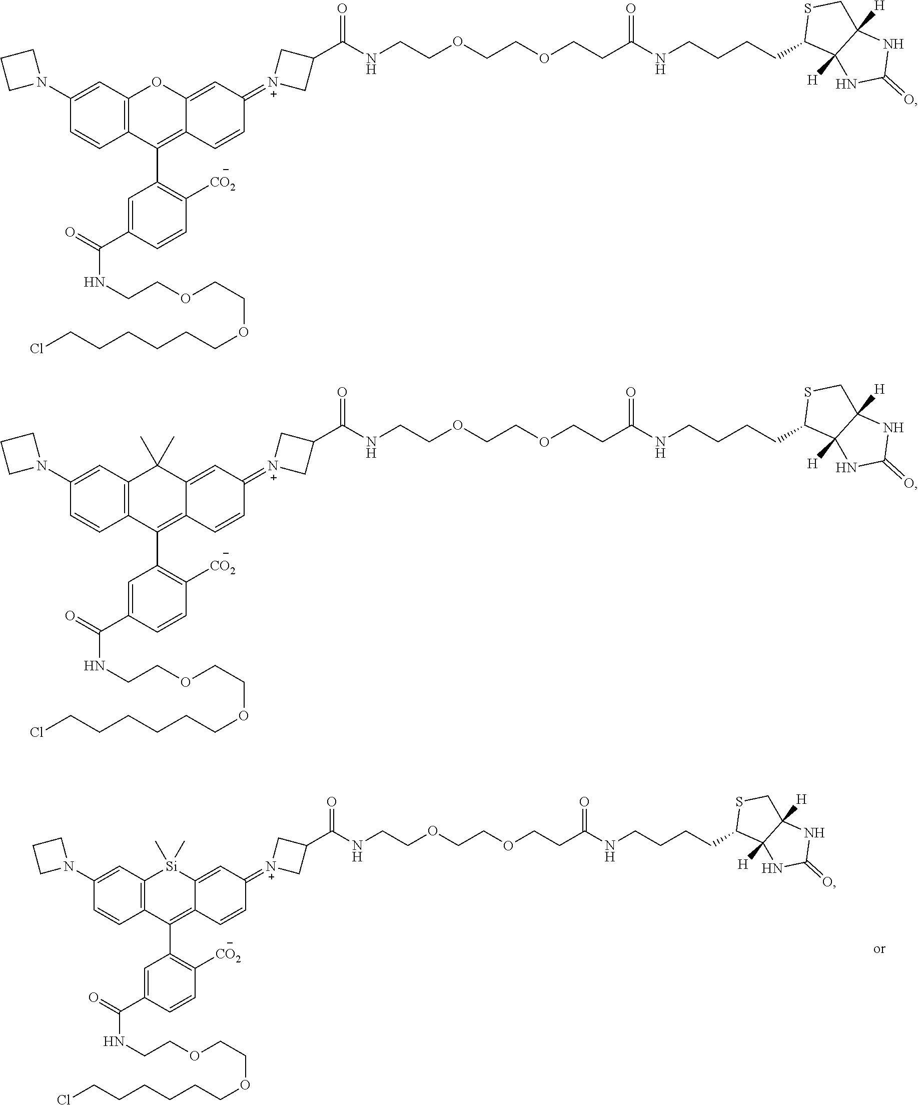

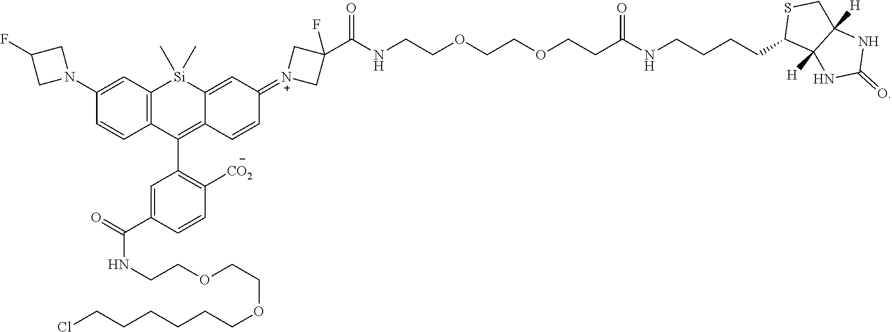

12. The compound of claim 1, of the following formula:

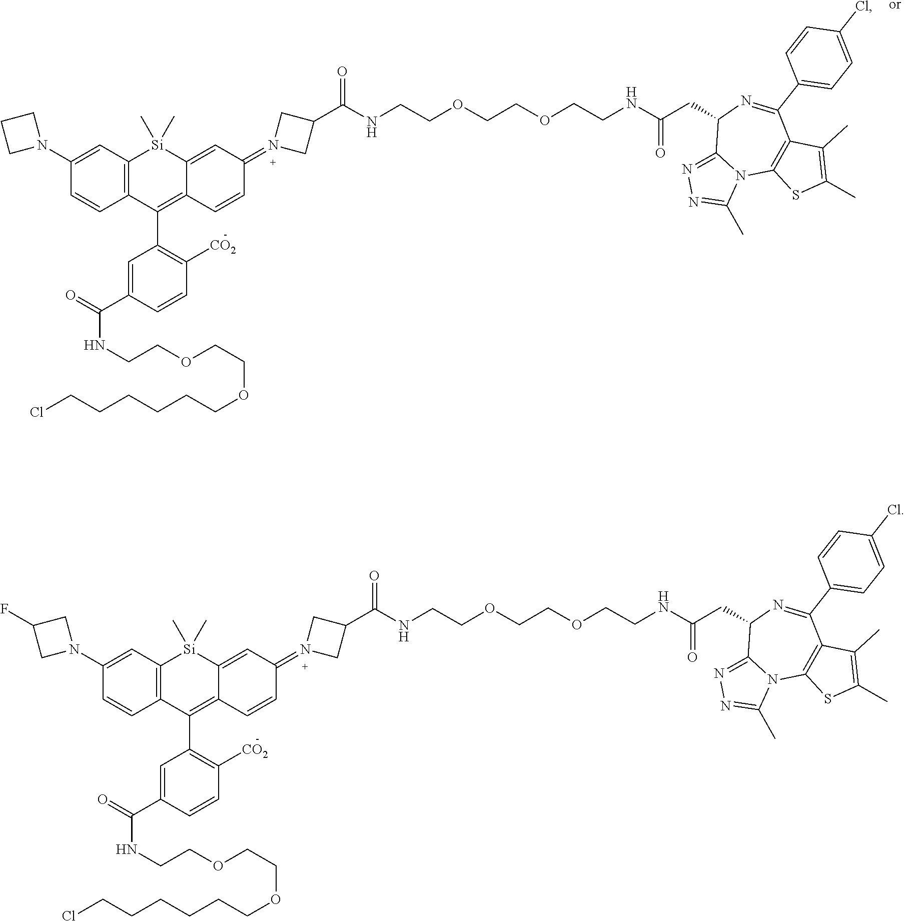

13. The compound of claim 1, of the following formula:

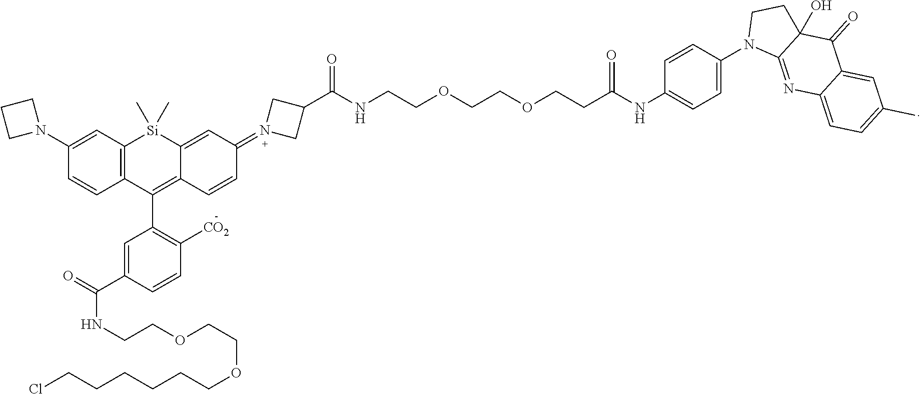

14. The compound of claim 1, wherein R¹ is an affinity tag-containing moiety consisting of an affinity tag and a linker joining the affinity tag to the compound.

15. The compound of claim 14, wherein the affinity tag is selected from the group consisting of biotin, desthiobiotin, trimethoprim/folate/methotrexate, a peptide epitope, or a click chemistry reagent.

16. The compound of claim 15, wherein the linker is selected from the group consisting of a polyethylene glycol (PEG) bearing a terminal amino group, an alkane bearing a terminal amino group, a polypeptide, or a maleimide-thiol group, or an azide-alkyne click chemistry group.

17. The compound of claim 1, wherein R¹ is a protein-manipulation moiety consisting of a protein-manipulation ligand and a linker joining the protein-manipulation ligand to the compound.

18. The compound of claim 17, wherein the protein-manipulation ligand is an inhibitor, allosteric binder, or activator.

19. The compound of claim 18, wherein the linker is selected from the group consisting of a polyethylene glycol (PEG) bearing a terminal amino group, an alkane bearing a terminal amino group, a polypeptide, or a maleimide-thiol group, or an azide-alkyne click chemistry group.

20. A complex, comprising: a compound of claim 1, and further comprising a self-labeling protein (SLP).

21. A method, comprising: contacting the compound of claim 1 and a self-labeling protein (SLP) with a cell; and visualizing fluorescence in the cell or purifying the SLP and associated biological components from the cell.
